(19) United States
(12) Patent Application Publication
(10) Pub. No.: US 2013/0133137 A1
(43) Pub. Date: May 30, 2013

(54) Patient positioning support structure with coordinated continuous nonsegmented articulation, rotation and lift, and locking fail-safe device

(71) Applicants: Roger P. Jackson, Prairie Village, KS (US); Lawrence E. Guerra, Mission, KS (US)

(72) Inventors: Roger P. Jackson, Prairie Village, KS (US); Lawrence E. Guerra, Mission, KS (US)

(21) Appl. No.: 13/694,392

(22) Filed: Nov. 28, 2012

Related U.S. Application Data
(60) Provisional application No. 61/629,815, filed on Nov. 28, 2011.

(51) Int. Cl. A61G 13/08; A61B 6/04

(52) U.S. Cl. CPC A61G 13/08; A61B 6/0407
USPC 5/617

(57) ABSTRACT

An apparatus for supporting a patient during a medical procedure with a breaking patient support subassembly and an angulation subassembly for changing patient angulation while substantially maintaining the height of the point of angulation. The angulation assembly includes an angulation wedge with upper and lower sides and a pair of opposed upper and lower guide members that slidably engage respective upper and lower sides. The guide members each include a key guide structure that slidably engages a complementary lock guide channel in the associated wedge upper and lower sides. The apparatus includes a torso trolley joined with the angulation subassembly and adapted for moving in cephalad and caudad directions in response to upward and downward breaking of the patient support subassembly.

### CROSS-REFERENCE TO RELATED APPLICATION

This patent application claims priority to U.S. Provisional Patent Application No. 61/629,815, filed Nov. 28, 2011, the specification of which is incorporated by reference herein in its entirety.

### BACKGROUND OF THE INVENTION

The present invention is directed to structure for use in maintaining a patient in a desired position during examination and treatment, including medical procedures such as imaging and surgery and in particular to such a structure that allows a surgeon to selectively position the patient for convenient access to the surgery site and providing for manipulation of the patient during surgery including the tilting, pivoting, angulating or bending of a trunk and/or a joint of a patient in a supine, prone or lateral position.

Current surgical practice incorporates imaging techniques and technologies throughout the course of patient examination, diagnosis and treatment. For example, minimally invasive surgical techniques, such as percutaneous insertion of spinal implants, involve small incisions that are guided by continuous or repeated intra-operative imaging. These images can be processed using computer software programs that produce three dimensional images for reference by the surgeon during the course of the procedure. If the patient support surface is not radiolucent or compatible with the imaging technologies, it may be necessary to interrupt the surgery periodically in order to remove the patient to a separate surface for imaging followed by transfer back to the operating support surface for resumption of the surgical procedure. Such patient transfers for imaging purposes may be avoided by employing radiolucent and other imaging compatible systems. The patient support system should also be constructed to permit unobstructed movement of the imaging equipment and other surgical equipment around, over and under the patient throughout the course of the surgical procedure without contamination of the sterile field.

It is also necessary that the patient support system be constructed to provide optimum access to the surgical field by the surgery team. Some procedures require positioning of portions of the patient's body in different ways at different times during the procedure. Some procedures, for example, spinal surgery, involve access through more than one surgical site or field. Since all of these fields may not be in the same plane or anatomical location, the patient support surfaces should be adjustable and capable of providing support in different planes for different parts of the patient's body as well as different positions or alignments for a given part of the body. Preferably, the support surface should be adjustable to provide support in separate planes and in different alignments for the head and upper trunk portion of the patient's body, the lower trunk and pelvic portion of the body as well as each of the limbs independently.

Certain types of surgery, such as orthopedic surgery, may require that the patient or a part of the patient be repositioned during the procedure while in some cases maintaining the sterile field. Where surgery is directed toward motion preservation procedures, such as by installation of artificial joints, spinal ligaments and total disc prostheses, for example, the surgeon must be able to manipulate certain joints while supporting selected portions of the patient's body during surgery in order to facilitate the procedure. It is also desirable to be able to test the range of motion of the surgically repaired or stabilized joint and to observe the gliding movement of the reconstructed articulating prosthetic surfaces or the tension and flexibility of artificial ligaments, spacers and other types of dynamic stabilizers before the wound is closed. Such manipulation can be used, for example, to verify the correct positioning and function of an implanted prosthetic disc, spinal dynamic longitudinal connecting member, interspinous spacer or joint replacement during a surgical procedure. Where manipulation discloses binding, sub-optimal position or even crushing of the adjacent vertebrae, for example, as may occur with osteoporosis, the prosthesis can be removed and the adjacent vertebrae fused while the patient remains anesthetized. Injury which might otherwise have resulted from a “trial” use of the implant post-operatively will be avoided, along with the need for a second round of anesthesia and surgery to remove the implant or prosthesis and perform the revision, fusion or corrective surgery.

There is also a need for a patient support surface that can be rotated, articulated and angulated so that the patient can be moved from a prone to a supine position or from a prone to a position and whereby intra-operative extension and flexion of at least a portion of the spinal column can be achieved. The patient support surface must also be capable of easy, selective adjustment without necessitating removal of the patient or causing substantial interruption of the procedure.

For certain types of surgical procedures, for example spinal surgeries, it may be desirable to position the patient for sequential anterior and posterior procedures. The patient support surface should also be capable of rotation about an axis in order to provide correct positioning of the patient and optimum accessibility for the surgeon as well as imaging equipment during such sequential procedures.

Orthopedic procedures may also require the use of traction equipment such as cables, tongs, pulleys and weights. The patient support system must include structure for anchoring such equipment and it must provide adequate support to withstand unequal forces generated by traction against such equipment.

Articulated robotic arms are increasingly employed to perform surgical techniques. These units are generally designed to move short distances and to perform very precise work. Reliance on the patient support structure to perform any necessary gross movement of the patient can be beneficial, especially if the movements are synchronized or coordinated. Such units require a surgical support surface capable of smoothly performing the multi-directional movements which would otherwise be performed by trained medical personnel. There is thus a need in this application as well for integration between the robotics technology and the patient positioning technology.

While conventional operating tables generally include structure that permits tilting or rotation of a patient support surface about a longitudinal axis, previous surgical support devices have attempted to address the need for access by providing a cantilevered patient support surface on one end. Such designs typically employ either a massive base to counterbalance the extended support member or a large overhead frame structure to provide support from above. The enlarged base members associated with such cantilever designs are problematic in that they can and do obstruct the movement of C-arm and O-arm mobile fluoroscopic imaging devices and other equipment. Surgical tables with overhead frame structures are bulky and may require the use of dedicated operating rooms, since in some cases they cannot be moved easily out of the way. Neither of these designs is easily portable or storable.

Thus, there remains a need for a patient support system that provides easy access for personnel and equipment, that can be easily and quickly positioned and repositioned in multiple planes without the use of massive counterbalancing support structure, and that does not require use of a dedicated operating room.

### SUMMARY OF THE INVENTION

The present invention is directed to a patient support system that permits adjustable positioning, repositioning and selectively lockable support of a patient's head and upper body, lower body and limbs in up to a plurality of individual planes while permitting tilting, rotating, angulation or bending and other manipulations aw well as full and free access to the patient by medical personnel and equipment. The system of the invention may be cantilevered or non-cantilevered and includes at least one support end or column that is height adjustable. The illustrated embodiments include a pair of opposed independently height-adjustable end support columns or piers. The columns may be independent or connected to a horizontally length-adjustable base. One support column according to the invention may be coupled with a wall mount or other stationary support. A patient support structure is connected to and bridges substantially between the pair of end supports, columns or piers. For example, in an embodiment according to the invention, the patient support structure is hingedly suspended between the end supports.

The patient support structure may be a frame or other patient support that is semi-constrained, having at least first and second hingeable or otherwise joined or connected portions, the first and second portions being selectively lockable in a first substantially planar orientation along a longitudinal axis of the support structure that resembles conventional constrained or fixed patient support structures. However, the hinged or semi-constrained support structure of the invention provides for the first and second portions that are also positionable and lockable in a plurality of angles with respect to one another, with each portion being movable to a position on either side of the first planar orientation. In other words, the patient support structure is capable of hinging or otherwise bending or “breaking” to form an angulation, articulation, break or joint, either upwardly or downwardly from a horizontal starting position and also when the support structure is in an inclined or declined position due to one of the support columns raising one end of the structure higher than another end. Furthermore, in addition to an “up” or “down” break, such a break or joint created by the two portions, the two portions may be oriented or rolled from side-to-side, as when the support structure is rotated about a longitudinal axis thereof, with or without the patient support structure being in a Trendelenburg position, a Reverse Trendelenburg position, or in a position that is non-parallel with respect to the floor.

In a first particular illustrated embodiment, articulation, jointing or breaking of the patient support structure at a somewhat central location between the pair of stationary end supports is supported by a cable drive system (tension band suspension). In another embodiment, a pull-rod assembly supports articulation to control and drive the “break” or articulation angle and render the patient support structure at a fixed degree of angulation. Such an embodiment further includes a length adjustment slider bar disposed at an end of the patient support, the patient support structure being supported by and slidingly movable along such slider bar with the bar following the angle of inclination of the patient support at such end. Other embodiments include cantilevered systems with connected or unconnected movable or telescoping base supports and other mechanisms for length adjustment. The first and second patient support structure portions may be in the form of frames, such as rectangular frames or other support structure that may be equipped with support pads for holding the patient, or other structure, such as imaging tops which provide a flat radiolucent surface.

The patient support structure and the support column or columns are coupled with respective rotation, articulation or angulation adjustment structure for positioning the first support portion with respect to a first column or end support and with respect to the second support portion and the second support portion with respect to the second column or end support. Rotation adjustment structure in cooperation with pivoting and height adjustment structure provide for the lockable positioning of the first and second patient support portions at a variety of selected positions and articulations with respect to the support columns including angulation coupled with Trendelenburg and reverse Trendelenburg configurations as well as providing for patient roll over in horizontal or tilted orientation. Lateral movement (toward and way from a surgeon) may also be provided by a bearing block feature. A pair of patient support structures (such as a support frame and an imaging table) may be mounted between end supports of the invention and then rotated in unison about a longitudinal axis to achieve 180° repositioning of a patient, from a prone to a supine position, for example.

In some embodiments of the invention, primary and secondary elevators are provide, for increasing the amount of angulation of the patient supported while simultaneously maintaining the patient's torso in a substantially horizontal position and distance or elevation above the floor. A failsafe lock may be mounted in the angulation subassembly to lock the position of the patient support in the event of catastrophic failure of the patient support structure. Movement of the patient's torso in concert with changes in angulation are provided by linkage of the angulation subassembly with a cephalad and caudal slidable torso support structure.

In a second particular illustrated embodiment, an apparatus for supporting and positioning a patient during a medical procedure, including a hinge subassembly joining a head portion of an open frame with a foot portion of a frame, the hinge subassembly being located about the middle of a patient supported on the frame and being adapted to controllably angle in both upwardly and downwardly directions and also to return to a non-angled position; and a hinge driver subassembly cooperating with the hinge subassembly, the hinge driver subassembly being translated in a cephalad direction when the hinge subassembly angles downwardly and also being translated in a caudad direction when the hinge subassembly angles upwardly, so as to manipulate a patient supported on the frame in a plurality of prone and non-prone positions, is provided. The hinge driver can be connected to the torso trolley subassembly described below.

In some further embodiments, the hinge driver subassembly is selectively lockable. In some further embodiment, the hinge driver subassembly includes a fail-safe locking structure adapted for locking the frame in the event of a catastrophic failure of the apparatus.

In another further embodiment, the apparatus includes a torso trolley subassembly associated with the frame head portion and adapted for supporting and translating the torso of the patient along the frame head portion in the cephalad and caudad directions and cooperating with the hinge subassembly and hinge driver subassembly to translate the torso in a cephalad direction when the hinge angles downwardly and also to translated the torso in a caudad direction when the hinge angles upwardly.

In yet another further embodiment, the apparatus includes a translation compensation subassembly located inside of the frame foot portion and cooperating with the hinge subassembly and the hinge driver subassembly to translate the foot portion in a cephalad direction when the hinge subassembly angles either upwardly or downwardly and also to translate the foot portion in a caudad direction and back to its starting point or position when the hinge subassembly moves toward the non-angled position.

In some further embodiments, the apparatus also includes a pair of space apart head and foot primary motorized vertical end elevators joined with outward ends of the frame head and foot portions; and an infinitely adjustable secondary motorized vertical end elevator adapted for better alignment and orientation of the frame so as to allow additional lowering of the outer end of the frame foot portion beyond an amount of lowering provided by the primary elevators.

In still some further embodiments, the hinge subassembly includes a maximum upward angulation of between about 40° and about 45°. In other embodiments, the hinge subassembly includes a maximum downward angulation of about 30°. In some embodiments, the hinge subassembly includes a total range of, motion of about 75°.

In a third particular illustrated embodiment of the invention, an apparatus for supporting a patient during a medical procedure, including an open frame having a single breaking location around the middle of a patient positioned on the frame, the frame being outwardly connected to and supported between spaced apart head and foot primary motorized vertical end elevators; and a secondary motorized vertical end elevator adapted for infinite adjustability to better align and orient the frame so as to allow additional lowering of a frame outer end beyond an amount of lowering provided by the primary elevators; wherein the frame can controllably break and bend, upwardly and downwardly, and is adapted to manipulate the patient in a plurality of selectively lockable prone and non-prone positions in cooperation with a frame or table base translation compensation mechanism located in-between the primary end elevators, while also cooperating with the primary and secondary motorized end elevators to move the patient vertically while the patient is positioned on the frame, is provided.

In a further embodiment, the frame translation compensation mechanism cooperatively moves an outer end of the frame away from the respectively connected end elevator in response to upward or downward bending of the frame at the frame breaking location; and the frame translation compensation mechanism cooperatively moves the outer end of the frame toward the respectively connected end elevator in response to unbending of the frame at the frame breaking location, so as to move the patient between at least two of the prone and non-prone positions. In some embodiments, the frame translation compensation mechanism is located within the frame.

In another further embodiment, the apparatus includes an angulation subassembly including a pivot associated with the frame breaking location and a driver; wherein downward bending of the frame breaking location translates the driver away from the frame breaking location and upward bending of the frame translates the driver toward the frame breaking location, so as to manipulate a patient supported on the frame in a plurality of prone and non-prone positions.

In yet another further embodiment, the apparatus also includes a chest slide or platform associated with a head end of the frame and tethered to the pivot or hinge; wherein upward bending of the frame translates the chest slide toward the pivot or hinge and downward bending of the frame translates the chest slide away from the pivot or hinge, so as to manipulate the patient in the plurality of prone and non-prone positions and without spinal distraction or compression, for example.

In still another further embodiment, the apparatus also includes a fail-safe mechanism adapted to substantially lock the frame in the event of a catastrophic failure of the apparatus.

In some further embodiment, the pivot includes a maximum upward bending position of between about 40° and about 45°. In some further embodiments, the pivot includes a maximum downward bending position of about 30°. In some further embodiments, the pivot includes a total range of motion of about 75°.

In a fourth particular illustrated embodiment of the invention, an apparatus for moving a patient between at least two of a plurality of selectively lockable prone and non-prone positions, the patient being supported on a frame connected outwardly to and supported between a pair of opposed primary vertical end elevators adapted to raise and lower a connected end of the frame, the frame including head and foot portions, the apparatus including a continuously adjustable angulation subassembly with a central pivot and located around a middle of the patient, the angulation subassembly being adapted to controllably break and angle the frame both upwardly and downwardly and to de-angle the frame to a position wherein the frame head and foot portions are located withing a single plane, wherein at least a portion of the angulation subassembly is translated in a cephalad direction when the angulation subassembly angles the frame downwardly and also is translated in a caudad direction when the angulation subassembly angles the frame upwardly; a chest slide subassembly supported by a head end of the frame and cooperating with the angulation subassembly to translate a torso of the patient in response to manipulation of the patient between at least two of a plurality of selectively lockable prone and non-prone positions; and a linkage tether connecting the angulation subassembly with the chest slide subassembly; wherein upward breaking of the angulation subassembly translates the chest slide toward the central pivot, and downward breaking of the angulation subassembly translates the chest slide away from the central pivot, is provided.

In a further embodiment, the apparatus includes an infinitely adjustable secondary vertical end elevator connected to and located between a primary vertical end elevator and a respective frame outer end, and adapted for lowering the respective frame outer end an additional distance beyond a distance of lowering provided by the respective primary vertical end elevator.

In another further embodiment, the apparatus includes a fail-safe apparatus adapted to lock the frame in the event of a catastrophic failure of the apparatus.

In still another further embodiment, the apparatus includes a frame translation compensation mechanism is located within the frame and adapted to translate the frame foot portion away from a connected end elevator in response to upward or downward angling of the frame and to translate the frame foot portion toward the connected end elevator in response to de-angling the frame.

In yet another embodiment, the central pivot includes a maximum upward breaking position of between about 40° and about 45°. In some embodiments, the central pivot includes a maximum downward breaking position of about 30°. In some embodiments, the central pivot includes a total range of motion of about 75°.

In yet another further embodiment, the chest slide subassembly includes a bracket slidingly engaging the frame and attached to the linkage tether; and a sliding chest support releasably engaging the bracket and supported by the frame. In a still further embodiment, the sliding chest support is an imaging table top.

In fifth illustrated embodiment, an apparatus for supporting a patient during a medical procedure, the apparatus including a breaking patient support subassembly including a head portion and a foot portion joined at a central pivot point; an angulation subassembly including: a pair of opposed hinges, each hinge having first and second knuckles with inboard ends pivotably joined by an upper axle located at the pivot point; a pair of opposed angulation wedges, each angulation wedge including upper and lower sides, and front and rear ends; a pair of upper guide members, each of the upper guide members slidably engaging the upper side of one of the angulation wedges, each of the upper guide members having a first through-bore that pivotably receives an associated upper axle therethrough; a pair of lower guide members, each lower guide member slidably engaging the lower side of the associated angulation wedge, each lower guide member having a second through-bore that pivotably receives an associated lower axle therethrough; and a pair of V-links having inner and outer ends, the lower axles pivotably joining the inner ends with a respective associated lower guide member, and each of the outer ends being pivotably joined with an outboard end of one of the associated first and second knuckles; a torso trolley slidingly supported by the head portion and joined with the angulation subassembly by a linkage tether, the torso trolley adapted for moving in cephalad and caudad directions with respect to the pivot point in response to upward and downward breaking of the patient support subassembly; a pair of selectively telescoping piers supporting the patient support subassembly, the pair of piers including: a head-end pier joined with the outboard end of the head portion and having an independently and continuously adjustable head-end primary elevator; and a foot-end pier joined with the outboard end of the foot portion and having independently and continuously adjustable foot-end primary and secondary elevators cooperating with the head-end primary elevator; and also a rotation subassembly cooperating with the head-end and foot-end piers, the elongate patient support subassembly and the angulation subassembly; and a powered actuator for actuating the angulation subassembly and for telescoping the head-end and foot-end piers, so as to so as to allow the hinges to move through an infinitely adjustable non-segmented plurality of angular orientations at the pivot point while substantially maintaining a selected height of the pivot point relative to a floor supporting the apparatus, is provided.

In a further embodiment, the apparatus includes a pair of tensioned rear tethers, each tether joining the actuator and an associated angulation wedge, so as to slidingly move the angulation wedges in the cephalad and caudad directions between the associated upper and lower sliding guide members, so as to cause the hinges to break upwardly or downwardly with respect to the floor.

In another further embodiment, the torso trolley includes a pair of opposed sliding brackets slidably engaging the head portion of the patient support subassembly; a pair of opposed sliding channel members removably received on the patient support subassembly and each of the sliding channel members releasably mating with a sliding bracket; and a chest slide removably and adjustably received on the pair of sliding channel members.

In yet another further embodiment, the apparatus includes a linkage strut pivotably joining each of the sliding brackets with the front end of an associated angulation wedge.

In still another further embodiment, the patient support subassembly includes a frame; and each of the sliding brackets includes an inner surface that defines a trapezoidal cross-section sized and shaped to slidingly mate with the associated frame, wherein the cross-section is taken perpendicular to a longitudinal axis of the frame.

In yet still another further embodiment, the apparatus also includes a fail-safe subassembly, the fail-safe subassembly having a toothed rack associated with the upper side of the angulation wedge and extending from about the front end of the angulation wedge to about the rear end thereof; a pawl pivotably linked with each of the upper sliders and including a ratchet tooth for engaging the toothed rack; wherein in the event of a catastrophic failure of the apparatus, the ratchet tooth engages the toothed rack, so as to block downward breaking of the patient support subassembly subsequent to the failure. In further embodiment, catastrophic failure includes at least one of mechanical failure and electrical failure.

In yet another further embodiment, the patient support subassembly includes a frame. In some further embodiments, the frame includes a trapezoidal cross-section, wherein the cross-section is taken perpendicular to a longitudinal axis thereof. In some further embodiments, the patient support subassembly further includes a second patient structure, the second structure being a solid support board. In some further embodiments, the patient support subassembly further includes a second patient structure, the second structure being an imaging table.

In another further embodiment of the apparatus, each angulation wedge includes at least one of an upper locking slot and a lower locking slot, wherein each of the locking slots extends between the front and rear ends thereof; and each of the locking slots being sized and shaped to slidingly receive therein a complementary mating guide key therein.

In a still further embodiment of the apparatus, each of the upper guide members includes an upper guide key sized and shaped to slidingly mate with an associated upper locking slot, so as to slidingly guide the associated upper guide member along the upper side of the associated angulation wedge; and each of the lower guide members includes a lower guide key sized and shaped to slidingly mate with an associated lower locking slot, so as to slidingly guide the associated lower guide member along the lower side of the associated angulation wedge. In a still further embodiment of the apparatus, each of the upper and lower locking slots includes a trapezoidal cross-section, wherein the cross-section is taken perpendicular to a longitudinal axis thereof.

In a further embodiment of the apparatus, the plurality of angular orientations includes a maximum upward breaking position of about 40° to about 45°. In another further embodiment of the apparatus, the plurality of angular orientations includes a maximum downward breaking position of about 30°. In still another further embodiment of the apparatus, the plurality of angular orientations includes a total range of motion of about 75°.

In a further embodiment, the apparatus includes a base; and a portion of an outboard end of the foot portion is lowered below a portion of the base, when the foot-end primary and secondary elevators are maximally lowered.

In another further embodiment of the apparatus, the rotation subassembly being adapted to rotate the patient support subassembly relative to a longitudinal axis thereof a distance selected from the group consisting of at least about ±5°, at least about ±10°, at least about ±15°, at least about ±20°, at least about ±25°, and at least about ±30°.

### OBJECTS AND ADVANTAGES OF THE INVENTION

Therefore, it is an object of the present invention to overcome one or more of the problems with patient support systems described above. Further objects of the present invention include providing breaking or hinged patient support structures; providing such structures wherein such break or joint may be in any desired direction; providing such structures that include at least one base support structure that allows for vertical height adjustment; providing such a structure wherein such base support is located at an end of the patient support, allowing for patient positioning and clearance for access to the patient in a wide variety of orientations; providing such a structure that may be rotated about an axis as well as moved upwardly or downwardly at either end thereof; providing such structure for cooperatively continuously and non-segmentedly changing the height and angulation of the patient support while moving the patient's torso so as to prevent excessive extension and compression of the patient's spinal column; providing such structure for maintaining the height of the point of angulation of the patient while simultaneously changing the amount of angulation thereof; and providing apparatus and methods that are easy to use and especially adapted for the intended use thereof and wherein the apparatus are comparatively inexpensive to make and suitable for use.

Other objects and advantages of this invention will become apparent from the following description taken in conjunction with the accompanying drawings wherein are set forth, by way of illustration and example, certain embodiments of this invention.

The drawings constitute a part of this specification and include exemplary embodiments of the present invention and illustrate various objects and features thereof.

### BRIEF DESCRIPTION OF THE DRAWINGS

FIG. 1 is a perspective view of a patient support structure according to the invention.

FIG. 2 is an enlarged and partial side elevational view of a portion of the support structure of FIG. 1.

FIG. 3 is an enlarged and partial top plan view of the support structure of FIG. 1.

FIG. 4 is an enlarged and partial perspective view of a portion of the structure of FIG. 1.

FIG. 5 is an enlarged and partial side elevational view of a portion of the structure of FIG. 1.

FIG. 6 is an enlarged and partial perspective view of a portion of the structure of FIG. 1.

FIG. 7 is an enlarged and partial perspective view of a first hinge of the structure of FIG. 1.

FIG. 8 is an enlarged and partial perspective view of a cooperating second hinge of the structure of FIG. 1.

FIG. 9 is an enlarged and partial elevational view of the hinge of FIG. 7.

FIG. 10 is an enlarged and partial perspective view of an outer portion of the hinge of FIG. 7 with portions broken away to show the detail thereof.

FIG. 11 is an enlarged and partial perspective view of an inner portion of the hinge of FIG. 7 with portions broken away to show the detail thereof.

FIG. 12 is an enlarged and partial perspective view of a portion of the structure of FIG. 1 showing a cable drive motor and winch cylinders.

FIG. 13 is a partial perspective view of a patient support frame of the structure of FIG. 1.

FIG. 14 is a partial perspective view of a patient imaging top for replacement with the patent support frame of FIG. 13.

FIG. 15 is a reduced perspective view of the structure of FIG. 1 shown with an imaging top of FIG. 14 replacing the support frame of FIG. 13 and shown in a planar inclined position.

FIG. 16 is a perspective view of the structure of FIG. 15 shown in a planar tilted position.

FIG. 17 is a perspective view of the structure of FIG. 15 shown in a planar inclined and tilted position.

FIG. 18 is a side elevational view of the structure of FIG. 15 shown in a symmetrical upward breaking position.

FIG. 19 is a side elevational view of the structure of FIG. 15 shown in a first inclined and upward breaking position.

FIG. 20 is a side elevational view of the structure of FIG. 15 shown in a second inclined and upward breaking position.

FIG. 21 is a side elevational view of the structure of FIG. 15 shown in a symmetrical downward breaking position.

FIG. 22 is a side elevational view of the structure of FIG. 15 shown in a first inclined and downward breaking position.

FIG. 23 is a side elevational view of the structure of FIG. 15 shown in a second inclined and downward breaking position.

FIG. 24 is an enlarged side elevational view of the structure of FIG. 1 shown in an upward breaking, inclined and tilted position.

FIG. 25 is a is a perspective view of a second embodiment of a patient support structure according to the invention including a patient support frame and an imaging table shown in a first spaced orientation.

FIG. 26 is a perspective view of the patient support structure of FIG. 25 shown tilted in an intermediate position during a rotation as would be used for a patient rollover.

FIG. 27 is a perspective view of the structure of FIG. 25 shown further tilted in a second intermediate position during rotation.

FIG. 28 is a perspective view of the structure of FIG. 25 shown after rotation to a final flipped position.

FIG. 29 is a perspective view similar to FIG. 25 showing the patient support frame and the imaging table in a second spaced orientation.

FIG. 30 is a front elevational view of a third embodiment of a patient support structure according to the invention.

FIG. 31 is a front elevational view of a fourth embodiment of a patient support structure according to the invention.

FIG. 32 is a perspective view of a fifth embodiment of a patient support structure according to the invention shown in a planar inclined position.

FIG. 33 is a perspective view of the structure of FIG. 32 shown in an inclined and upward breaking position.

FIG. 34 is a perspective view of the structure of FIG. 32 shown in a substantially symmetrical downward breaking position.

FIG. 35 is a reduced side elevational view of a sixth embodiment of a patient support structure according to the invention shown in a substantially horizontal and planar position.

FIG. 36 is a reduced side elevational view of the structure of FIG. 35 shown in a symmetrical downward breaking position.

FIG. 37 is a reduced side elevational view of the structure of FIG. 35 shown in a symmetrical downward breaking position.

FIG. 38 is an enlarged and partial top plan view of a portion of the structure of FIG. 35 and shown in the same position as shown in FIG. 35.

FIG. 39 is an enlarged and partial side elevational view of the structure of FIG. 35 and shown in the same position as shown in FIG. 35.

FIG. 40 is an enlarged and partial side elevational view of the structure of FIG. 35 and shown in the same position as shown in FIG. 35.

FIG. 41 is an enlarged and partial perspective view of the structure shown in FIG. 40.

FIG. 42 is an enlarged and partial top plan view of a portion of the structure of FIG. 35 and shown in the same position as shown in FIG. 36.

FIG. 43 is an enlarged and partial side elevational view of the structure of FIG. 35 and shown in the same position as shown in FIG. 36.

FIG. 44 is an enlarged and partial side elevational view of the structure of FIG. 35 and shown in the same position as shown in FIG. 36.

FIG. 45 is an enlarged and partial top plan view of a portion of the structure of FIG. 35 and shown in the same position as shown in FIG. 37.

FIG. 46 is an enlarged and partial side elevational view of the structure of FIG. 35 and shown in the same position as shown in FIG. 37.

FIG. 47 is an enlarged and partial side elevational view of the structure of FIG. 35 and shown in the same position as shown in FIG. 37.

FIG. 48 is a side elevational view of another embodiment of the patient support structure according to the invention, shown in a substantially horizontal and planar position.

FIG. 49 is a side elevation view of the patient support structure of FIG. 48, shown in a downward breaking position and in a fully elevated position.

FIG. 50 is a side elevation view of the patient support structure of FIG. 48, shown in an upward breaking position and in a fully lowered position.

FIG. 51 is an enlarged bottom perspective view of a portion of the patient support structure of FIG. 48, and shown in the same position as shown in FIG. 48.

FIG. 52 is an enlarged bottom perspective view of a portion of the patient support structure of FIG. 48, shown in the same position as shown in FIG. 49.

FIG. 53 is an enlarged bottom perspective view of a portion of the patient support structure of FIG. 48, shown in the same position as shown in FIG. 50.

FIG. 54 is an enlarged partial perspective view of the patient support structure of FIG. 48, shown in a fully elevated position.

FIG. 55 is an enlarged partial perspective view of the patient support structure of FIG. 54, shown in a fully lowered position.

FIG. 56 is a side perspective view of the patient support structure of FIG. 52, shown in a downward breaking position and a fully lowered position.

FIG. 57 is an enlarged top elevational view of the patient support structure of FIG. 48, shown in the same position as shown in FIG. 49.

FIG. 58 is an enlarged cross-sectional view of a portion of the patient support structure of FIG. 57, taken along line 58-58 of FIG. 57, and shown in the same position as shown in FIG. 48.

FIG. 59 is an enlarged cross-sectional view of a portion of the patient support structure of FIG. 48, taken along line 58-58 of FIG. 57, and shown in the same position as shown in FIG. 49.

FIG. 60 is an enlarged cross-sectional view of a portion of the patient support structure of FIG. 48, taken along line 58-58 of FIG. 57, and shown in the same position as shown in FIG. 50.

FIG. 61 is an enlarged foot-end elevational view of the patient support structure of FIG. 48 and shown in the same position as shown in FIG. 49.

FIG. 62 is an enlarged head-end elevational view of the patient support structure of FIG. 48 and shown in the same position as shown in FIG. 49.

FIG. 63 is a side elevation view of the patient support structure of FIG. 48, shown in an upwardly breaking position and in a fully elevated position.

FIG. 64 is an enlarged top perspective view of a portion of the hinge and roller of FIG. 48 and in a downward breaking position.

FIG. 65 is an enlarged bottom perspective view of the hinge and roller of FIG. 64.

FIG. 66 is an enlarge perspective view of the patient support subassembly of the patient support structure of FIG. 48 with portions broken away and portions shown in phantom to show detail thereof.

FIG. 67 is an enlarged perspective view of the gearbox of the patient support structure of FIG. 48 with portions removed to show detail thereof.

FIG. 68 is an enlarged partial perspective view of portions of the tensioned angulation subassembly of the patient support structure of FIG. 48, including the upper and lower rollers and failsafe structure.

FIG. 69 is an enlarged partial side view of portions of the tensioned angulation subassembly of the patient support structure of FIG. 48, including the upper and lower rollers and failsafe structure.

FIG. 70 is an enlarged partial perspective view of portions of another tensioned angulation subassembly of the patient support structure FIG. 48, including upper and lower guide members and another failsafe structure.

FIG. 71 is an enlarged exploded view of the angulation wedge, upper and lower guide members and pawl of FIG. 70.

FIG. 72 is an enlarged side view of the assembly of FIG. 70, with portions cut away to show detail thereof.

FIG. 73 is an enlarged perspective view of the torso trolley and head-end frame of FIG. 48, with portions cut away to show detail thereof.

FIG. 74 is an enlarged cross-sectional view of the torso trolley of FIG. 73, taken along line 74-74 of FIG. 73, and shown in the same position as shown in FIG. 73.

FIG. 75 is an enlarged perspective view of the assembly of FIG. 48, with the torso trolley removed.

FIG. 76 is an enlarged perspective view of the assembly of FIG. 48.

FIG. 77 is a perspective view of the assembly of FIG. 76, including a solid operating board or imaging table.

FIG. 78 is a perspective view of a patient support structure in another embodiment.

FIG. 79 is a perspective view of a patient support structure of FIG. 78.

FIG. 80 is a perspective view of a patient support structure of FIG. 78, including an imaging table top.

FIG. 81 is a perspective view of a patient support structure of FIG. 78, including a wide imaging table top.

### DETAILED DESCRIPTION OF THE ILLUSTRATED EMBODIMENTS

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

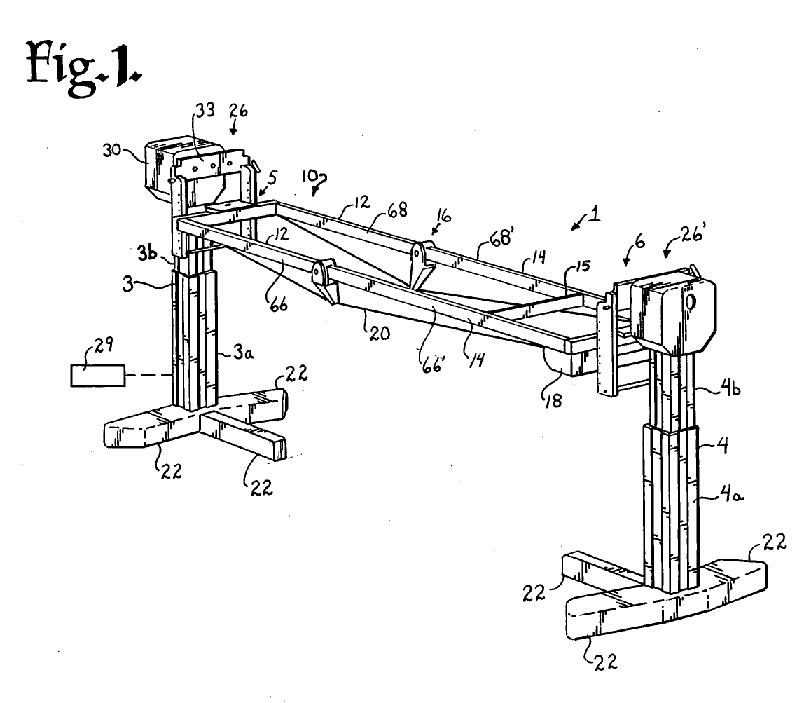

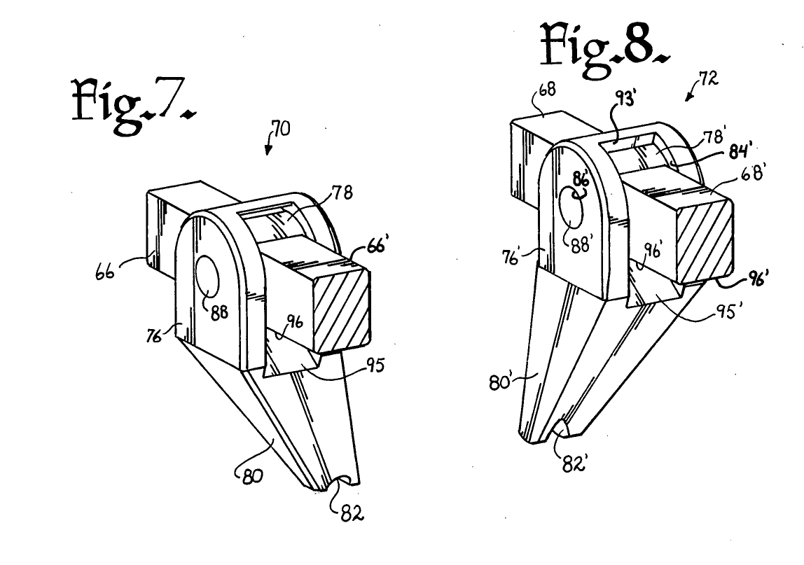

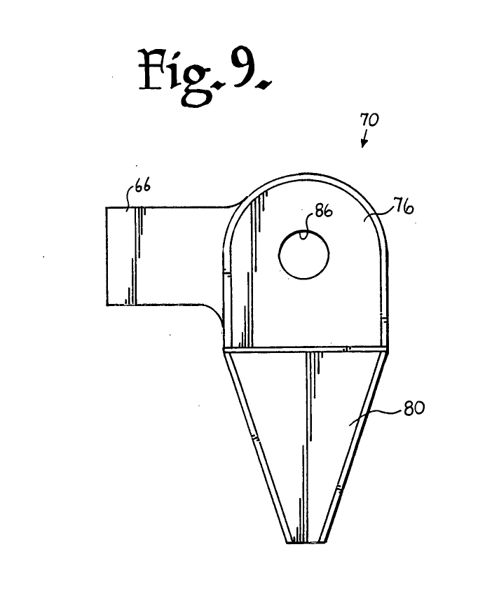

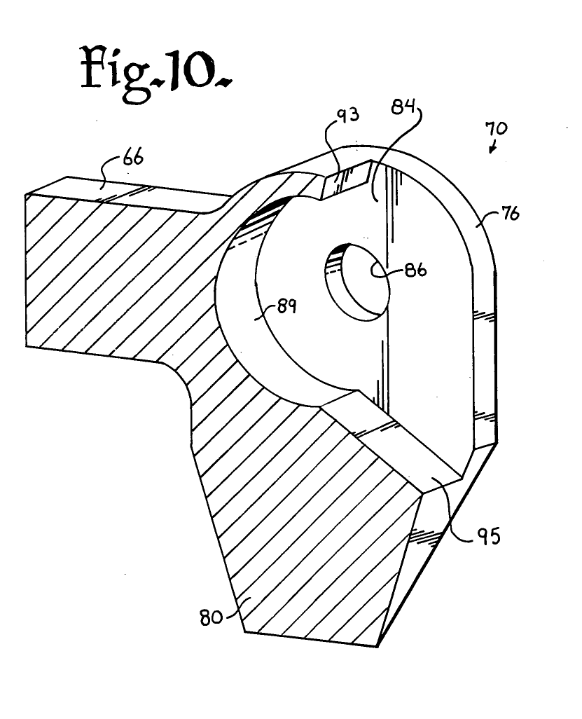

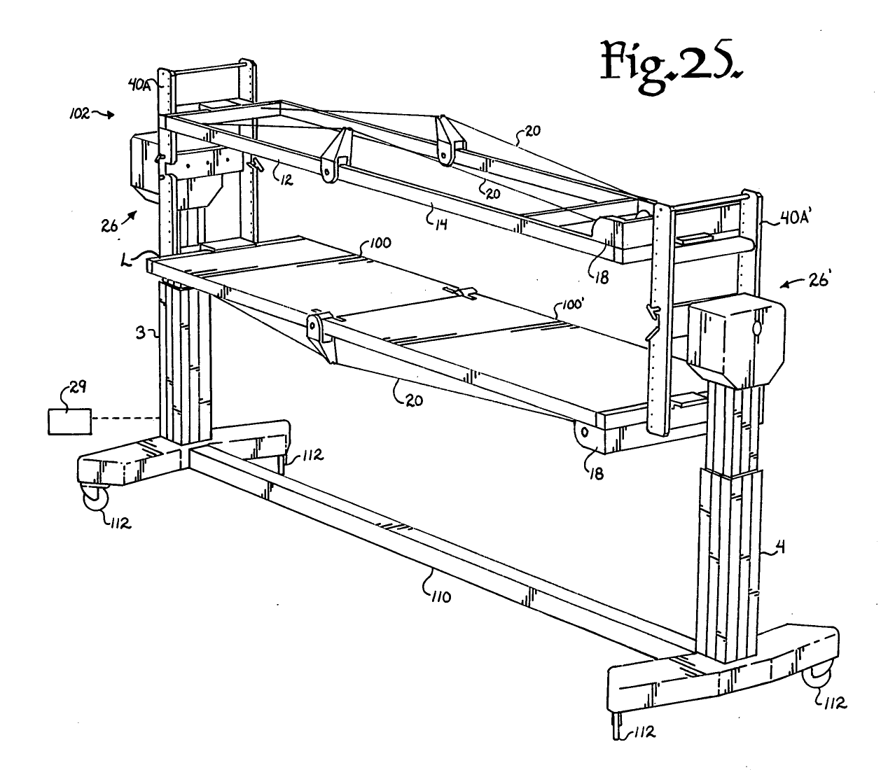

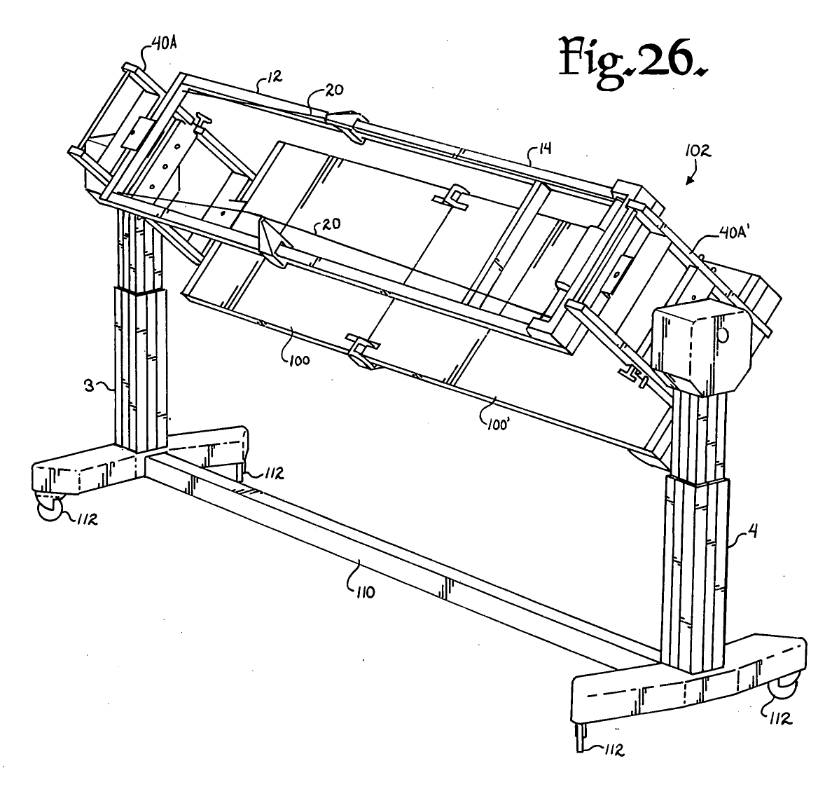

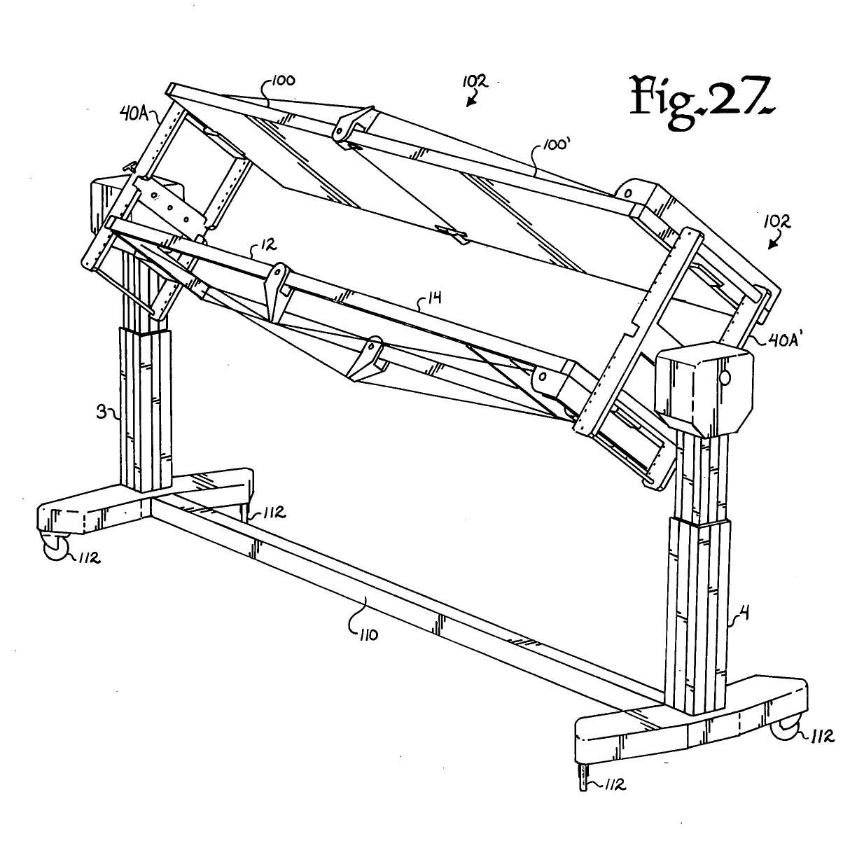

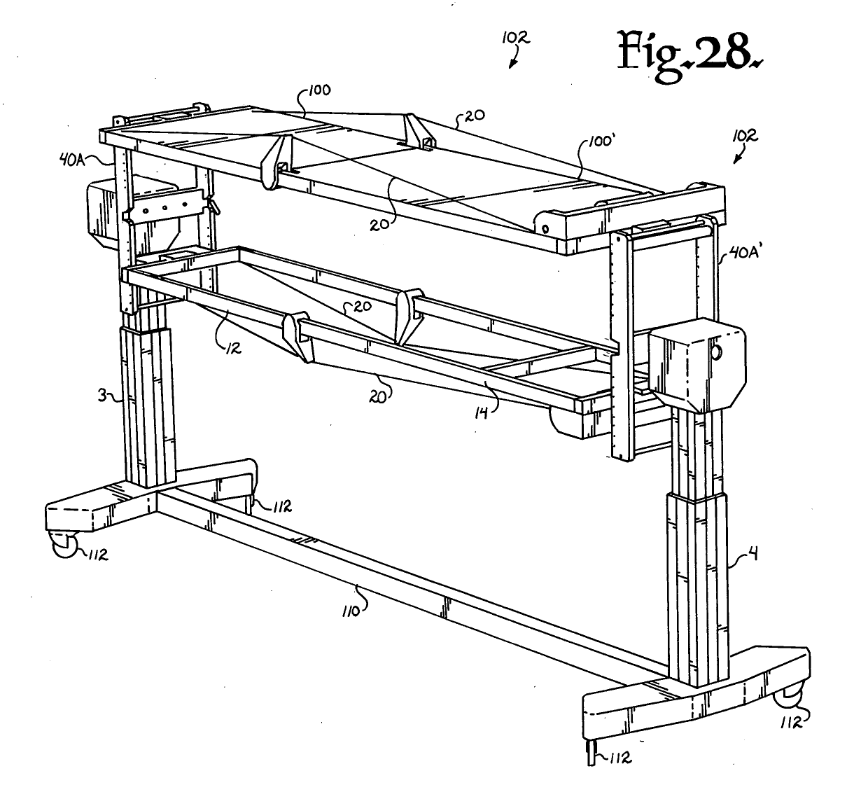

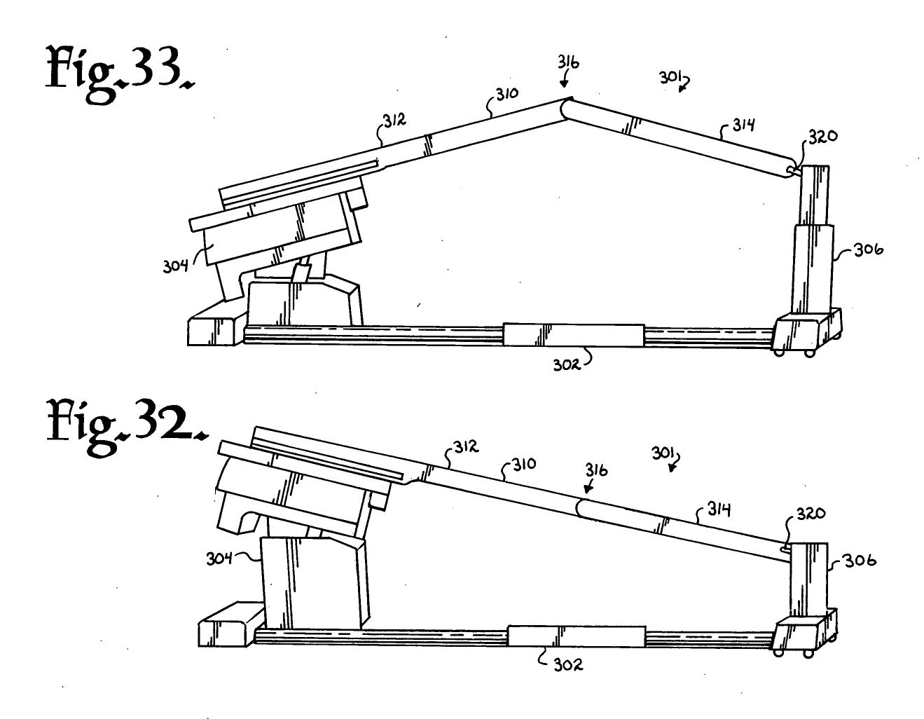

Referring now to the drawings, a patient positioning support structure according to the invention is generally designated by the reference numeral 1 and is depicted in FIGS. 1-12. The structure 1 includes first and second upright support piers or columns 3 and 4 which are illustrated as independent, stationary floor base support structures as shown in FIG. 1 or may be connected to one another by a non-telescoping base support as illustrated in the embodiment shown in FIGS. 25-28. In some embodiments according to the invention as shown, for example, in FIGS. 32-34, the base connection places the columns in a selectively telescoping relationship. It is also foreseen that in certain embodiments according to the invention, one of the support columns may be replaced by a conventional operating room table, or may even be a wall mount. In the first illustrated embodiment, the upright support column 3 is connected to a first support assembly, generally 5, and the upright support column 4 is connected to a second support assembly, generally 6. Between them, the support assemblies 5 and 6 uphold a removable elongate, articulate jointed or breaking patient holding or support structure, generally 10 and optionally, a second removable patient support structure that will be described with respect to another embodiment of the invention. The illustrated support structure 10 includes a first frame section 12, a second frame section 14 with a transverse support cross bar 15, and a pivot or hinge assembly, generally 16. In the illustrated embodiment, the pivot assembly further includes a cable drive system including a dual winch 18 and cooperating cables 20.

The columns 3 and 4 are supported by outwardly extending feet 22 that may or may not include spaced apart casters or wheels (not shown) each equipped with a floor-lock foot lever for lowering the feet 12 into a floor-engaging position as shown in FIG. 1. The columns 3 and 4 each include two or more telescoping lift arm segments 3a, 3b and 4a, 4b, respectively that permit the height of each of the columns 3 and 4 to be selectively increased and decreased in order to raise and lower all or a selected portion of the connected patient support structure 10. It is foreseen that the vertical supports 3 and 4 may be constructed so that the column 3 has a greater mass than the support column 4 or vice versa in order to accommodate an uneven weight distribution of the human body. Such reduction in size at the foot end of the system 1 may be employed in some embodiments to facilitate the approach of personnel and equipment.

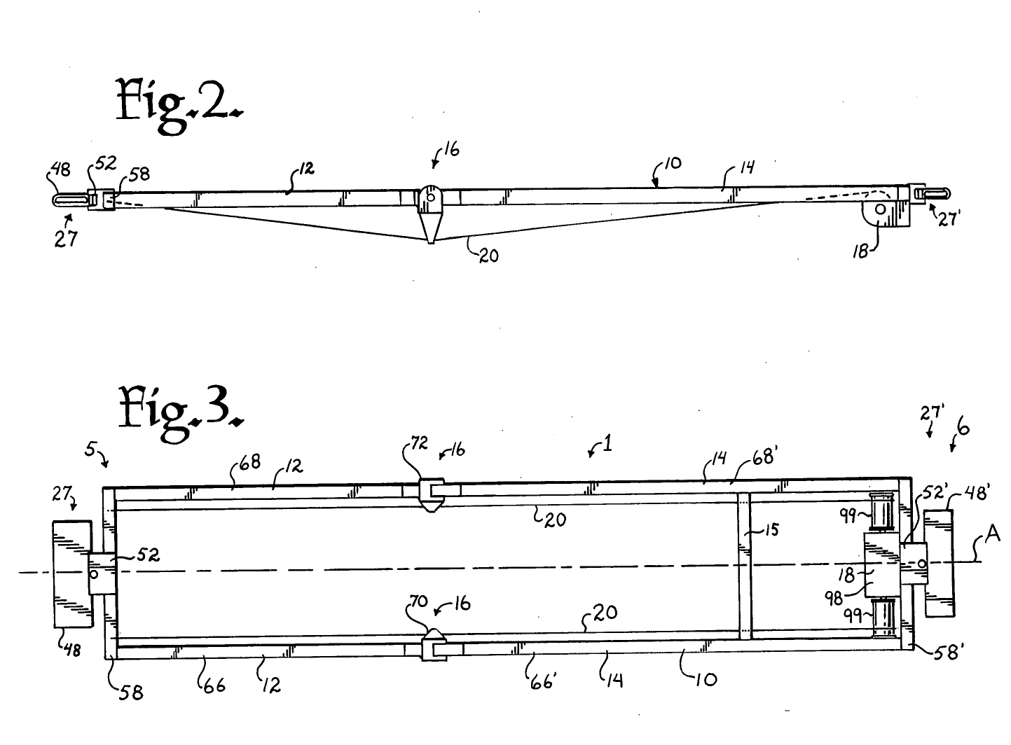

Each of the support assemblies 5 and 6 generally includes a rotation subassembly 26 and 26′ and an angulation subassembly 27 and 27′, respectively, that are interconnected as will be described in greater detail below and include associated power source and circuitry linked to a controller 29 (FIG. 1) for cooperative and integrated actuation and operation. The rotational subassemblies 26 and 26′ enable coordinated rotation of the patient support structure 10 about a longitudinal axis of the structure 1. The angulation subassemblies 27 and 27′ shown in FIGS. 2 and 3 enable the selective hinging, articulation or breaking of the support 10 at the hinge assembly 16 at desired levels and increments as well as selective tilting of the frame portions 12,14 with respect to a longitudinal axis of such frame portion.

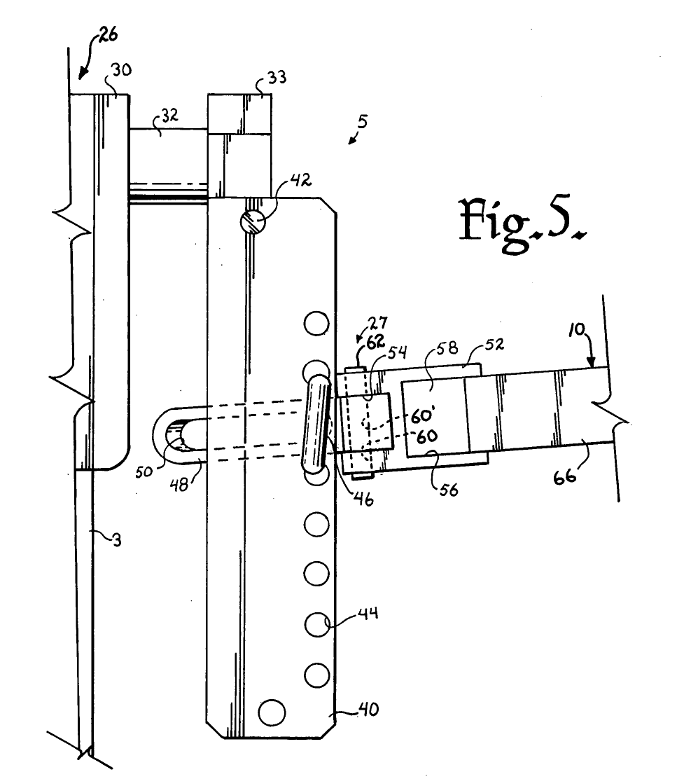

The rotation subassembly or mechanism 26, shown in FIGS. 1 and 5, includes at least one motor housing 30 surmounting the support column 3. In the illustrated embodiment, only one rotational motor is provided, but it is foreseen that a cooperating motor may also be mounted on the support column 4. A main rotational shaft 32 extends from the motor housing 30 that turns a rotation structure 33. The rotation structure 33 in turn rotates the connected patient support 10 about a longitudinal axis as will be described in greater detail below. The motor housing 30 contains a rotary electric motor or other actuator drivingly engaged with the shaft 32. The rotation mechanism 26 is operated by actuating the motor using a switch or other similar means. The rotation structure 33 is fixed to the shaft 32 at a location spaced from the motor housing 30 and the support column 3 to provide clearance for rotation of the connected patient support structure 10.

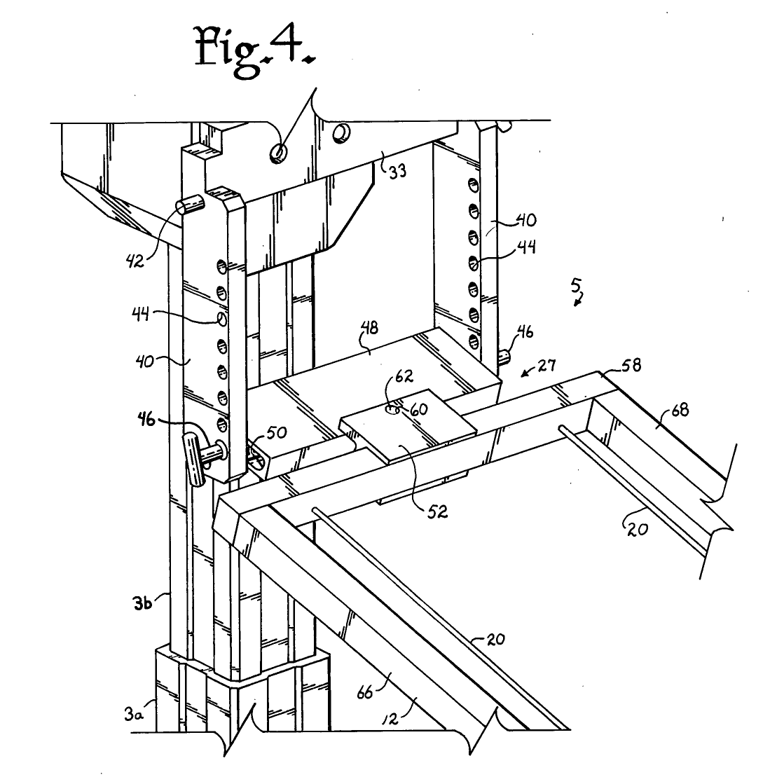

As shown in FIGS. 4 and 5, the rotation structure 33 is attached to a pair of translation posts or H-bar posts 40 disposed at either end of the rotation structure 33. The posts 40 are each attached to the structure 33 by a pin 42, bolt, or other fixing structure. A plurality of cooperating apertures 44 formed in the posts 40 provide passageway for a pivot pin 46 to extend therethrough. The pivot pin 46 is receivable in each cooperating pair of apertures 44 allowing for selective placement of a translation connector 48 that is sized and shaped to be received between the pair of posts 40 and also receive the pivot pin 46 therethrough. The pin 46 and connector 48 are thus positionable in an orientation transverse to the longitudinal extension of the support 10 at a variety of heights to be selected by the surgeon and readily changeable, even during surgery if necessary, to vary the height of the frame section 12. The multiple location or height feature is also advantageous when more than one frame or patent structure is mounted in tandem as shown, for example in FIGS. 25-29. The position of the frame or other structure may be desirably changed to provide close proximity to an imaging top with a distance between a patient support and an imaging top being expandable or reduceable depending upon the size or other attributes of a patient and surgical or other requirements. As illustrated in FIG. 5, the connector 48 has a slot 50 for receiving the pivot pin 46.

Also with reference to FIGS. 4 and 5, the translation connector 48 is in turn attached to a pivot connector 52. The pivot connector 52 includes first and second outwardly opening and opposed slots 54 and 56. The first slot 54 is sized and shaped for receiving the translation connector 48 and the second slot is sized and shaped for receiving an end connection 58 of the frame section 12. The pivot connector 52 further includes a through aperture or bore 60 running substantially perpendicular to the slot 54 and communicating therewith. The aperture 60 is sized and shaped to receive a pivot pin 62 therethrough. The connector 48 also includes a through bore 60′ that receives the pivot pin 62. The swivelable connection provided by the pin 62 allows for some forward and rearward lateral movement of the attached frame end connection 58 and thus the frame section 12, providing a degree of freedom and clearance needed for rotation the patient support about a longitudinal axis of a patient. The slot 56 is sized and shaped to frictionally engage the frame end connection 58, thus securely fixing the end connection 58 to the pivot connector 52. The frame end connection 58 is in turn fixed to each of elongate frame members 66 and 68 of the frame section 12. The frame members 66 and 68 are each hingedly connected to the hinge assembly 16 to be described in greater detail below. Pivoting of the translation connector 48 with respect to the pin 46 provides for selected articulation of the frame section 12 (that includes the end connection 58 and the frame members 66 and 68) and/or the entire support 10 with respect to the support pier or column 3.

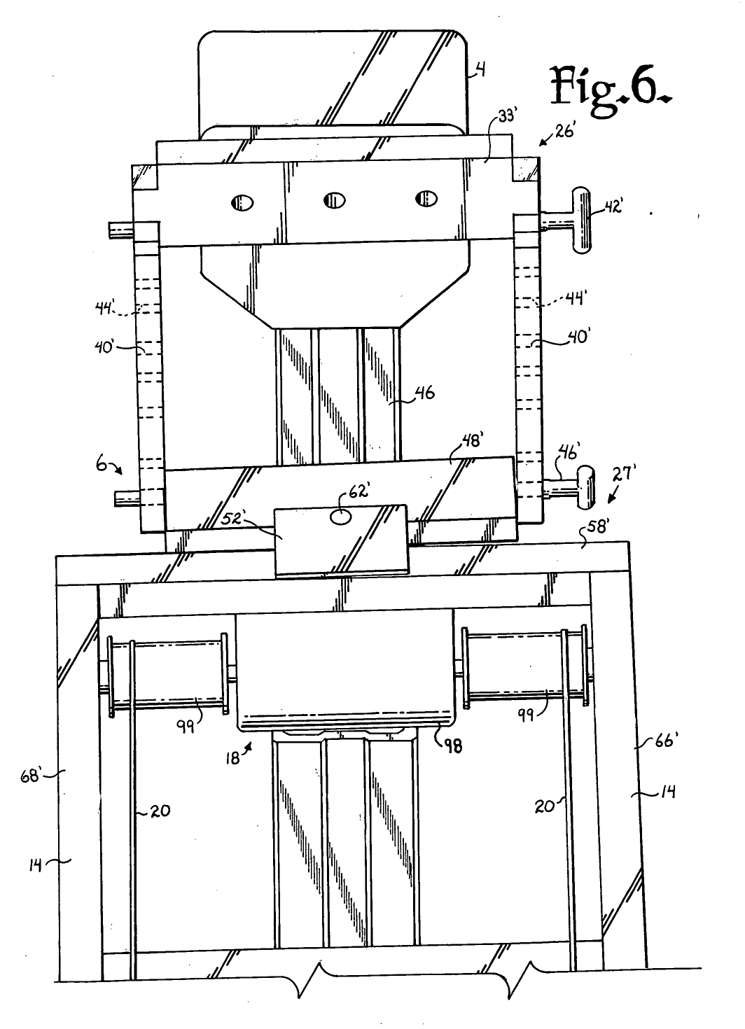

With reference to FIG. 6, at the support pier or column 4, the support assembly 6 is substantially similar to the support assembly 5 with the exception that the rotation subassembly 26′ can be passive and, therefore, not include a motor. However, the support pier or column 4 preferably includes a powered mechanism to provide selective height adjustment of the subassembly 26′. A rotation structure 33′ is spaced from and freely rotatable with respect to the column 4. The structure 33′ includes a shaft (not shown) extending outwardly therefrom similar to the rotation shaft 32, the shaft being rotatingly received in an aperture in the support column 4.

The rotation subassembly 26′ and the angulation subassembly 27′ otherwise include elements identical to or substantially similar to the elements of the subassemblies 26 and 27. Specifically, H-bar posts 40′, pin 42′, apertures 44′, pivot pin 46′, translation connector 48′, slot 50′, pivot connector 52′, end connector 58′ and pivot pin 62′, are identical or substantially similar in form and cooperate with other elements identically or substantially similarly to what has been described previously herein with respective H-bar posts 40, pin 42, apertures 44, pivot pin 46, translation connector 48, slot 50, pivot connector 52, end connector 58 and pivot pin 62.

The frame 14 further includes frame members 66′ and 68′ that are each fixed to the end connector 58′. The frame members 66′ and 68′ are pivotally or hingedly connected to respective frame members 66 and 68 by the hinge assembly 16. Specifically, the frame member 66 is attached to the frame member 66′ by the hinge mechanism 70 and the frame member 68 is attached to the frame member 68′ by the hinge mechanism 72.

With particular reference to FIGS. 3, 7 and 9-11, the hinge mechanism 70 includes an outer member 76 and an inner member 78. The outer member 76 is fixed or may be integral with the elongate frame member 66, while the inner member 78 is integral or otherwise fixed to the frame member 66′. The outer member 76 further includes an extension 80 with a groove 82 for receiving and guiding the cable 20. The extension 80 tapers in a direction from the outer member interior 84 to the groove 82. The extension 80 is configured to cause a slight upward break or bend of the support 10 when the extension 80 comes into contact with the cable 20 at the groove 82. In that way, when the cables 20 are reeled in to shorten the hypotenuse of the triangle formed by the cable, the section 12 and the section 14, the sections 12 and 14 move toward one another, resulting in the upward break as illustrated, for example, in FIG. 18. The downward break or joint illustrated, for example, in FIG. 21 is a result of lengthening the cable 20 distance and allowing gravity to drop the hinge 70. The extension 80 is shaped to extend slightly inwardly toward a longitudinal axis A of the support 10, thereby guiding the cable 20 along a path within a periphery of the frame sections 12 and 14 when the extension 80 is in contact with the cable 20 when in a downward breaking configuration directed toward the cable with the cable 20 being received at the groove 82.

It is foreseen that if an exclusively upward breaking or jointing embodiment is desired according to the invention, the sections 12 and 14 may be positioned with respect to two end columns to always include a slight upward break, joint or bend at the hinge or pivot between the sections 12 and 14. When the telescoping base is actuated to move the columns toward one another, the sections 12 and 14 would automatically further break or articulate upwardly and toward one another. Downward breaking or jointing would not be possible in such an embodiment as the maximum distance between the two end columns would still ensure a slight upward break or hinge between the sections 12 and 14. Such an embodiment would be acceptable for use because patient holding pads could be positioned on the frames 12 and 14 such that the patient would be in a substantially horizontal position even when there is a slight upward bend or break at the hinge between the sections 12 and 14.

Returning to the hinge 70 of illustrated embodiment, the inner member 78 is slidingly and rotatably receivable in an interior 84 of the outer member 76. The outer member has a pair of pivot apertures 86 and the inner member has a pivot aperture 87, the apertures cooperating to create a through bore for receiving a pivot pin 88 through both the inner and outer hinge members. The interior 84 includes a curved partially cylindrical surface 89 for slidingly receiving a cooperating outer rounded and partially cylindrical surface 90 of the inner member 78. The inner member 78 further includes a downward breaking stop or projection 92 that limits a downward pivot (in a direction toward the cables 20) of the hinge 70 in the event the cables 20 should fail. The stop 92 abuts against a surface 93 of the interior 84. In the illustrated embodiment, the stop 92 limits the extent of rotation or hinging of the section 66 with respect to the section 66′ to about twenty-five degrees. Upward pivot (in a direction away from the cables 20) is limited by abutment of an inner planar surface 95 with a planar surface 96 of the hinge inner member 78.

With particular reference to FIG. 8, the hinge mechanism 72 is substantially a mirror image of the hinge mechanism 70 and therefore includes the following elements: a hinge outer member 76′, an inner member 78′, an extension 80′ with a groove 82′, an interior 84′, pivot apertures 86′, a pivot pin 88′, a curved surface 89′ (not shown), an outer surface 90′ (not shown), a stop 92′ (not shown), an abutment surface 93′, an inner planar surface 95′ and a planar surface 96′ that are identical or substantially similar in shape and function to the respective hinge outer member 76, inner member 78, extension 80, groove 82, interior 84, pivot apertures 86, pivot pin 88, curved surface 89, outer surface 90, stop 92, abutment surface 93, inner planar surface 95 and planar surface 96 described herein with respect to the hinge 70.

It is noted that other hinge or pivot mechanisms may be utilized in lieu of the hinge assembly 16. For example, the polyaxial joint 95 illustrated and described in Applicant's U.S. Pat. No. 7,152,261 and pending U.S. patent application Ser. No. 11/159,494 filed Jun. 23, 2005, may be incorporated into the patient support structure 10 at the break or joint between the sections 12 and 14. The disclosures of U.S. Pat. No. 7,152,261 and U.S. patent application Ser. No. 11/159,494 are incorporated by reference herein. It is foreseen that a rotating universal joint operated type of hinge mechanism could be used with the invention, etc.

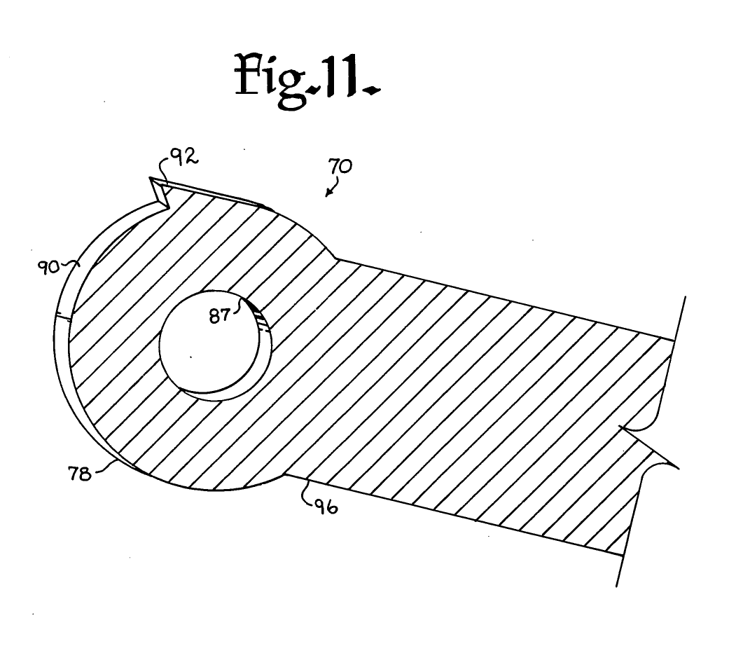

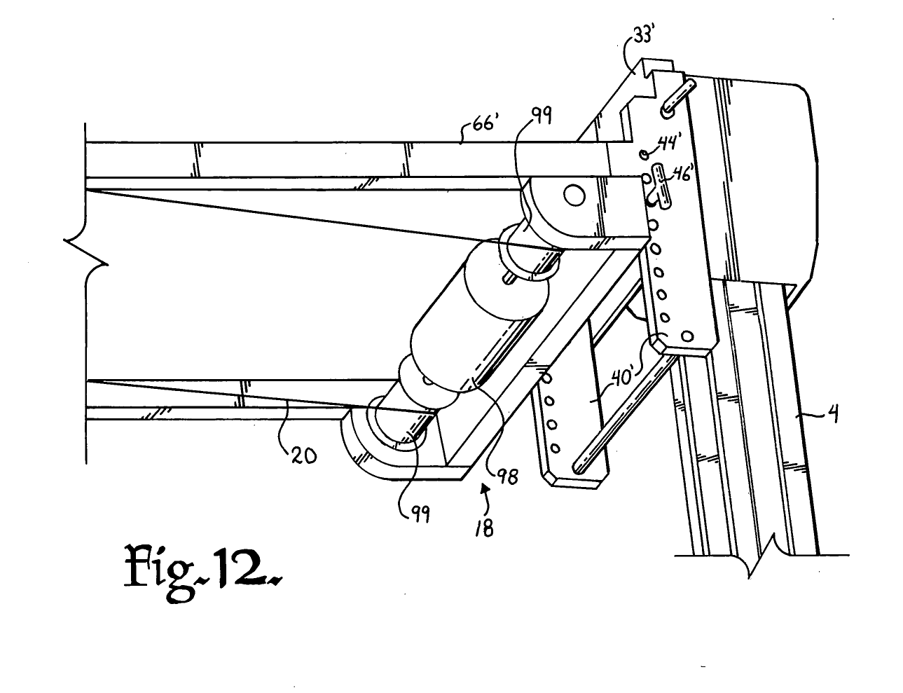

With particular reference to FIGS. 6 and 12, the cable drive system 18 includes a rotary motor 98 cooperating with and driving by rotation a pair of winch cylinders 99 disposed on either side of the motor 98. The motor 98 and cylinders 99 are mounted to the end connector 58′ located near the support column 4. Each cable 20 is attached to one of the winch cylinders 99 at one end thereof and to the end connector 58 at the other end thereof. In a first longitudinal position wherein the section 12 is substantially planar with the section 14, the cables 20 are wound about the winch cylinders 99 an amount to provide enough tension in the cables 20 to maintain such a substantially planar orientation and configuration, with the hinge extensions 82 and 82′ being in contact with each of the cables 20. The motor 98 is preferably low speed and high torque for safely winding both of the cables 20 simultaneously about the cylinders 99 to draw the section 12 toward the section 14 to result in an upward breaking or jointing configuration with the hinges 70 and 72 disposed in spaced relation with the cables 20 and the hinges 70 and 72. The motor 98 may be reversed, reversing the direction of rotation of the winch cylinders 99 for slowly unwinding the cables 20 to a downward breaking or jointing configuration. As the cables 20 unwind, gravity draws the support sections 12 and 14 downward with the cables 20 being received in the grooves 82 and 82′ of the hinge extensions 80 and 80′. As the cables 20 slacken, the hinges 70 and 72 continue to lower pressing down upon the cables 20.

It is noted that the frame sections 12 and 14 are typically equipped with pads (not shown) or other patient holding structure, as illustrated, for example, in Applicant's U.S. Pat. No. 5,131,106, the disclosure of which is incorporated by reference herein. It is foreseen that such patient holding structure could translate or glide along the frame sections 12 and 14. Furthermore, with respect to FIGS. 13 and 14, the frame member sections 66 and 68 of section 12 and the frame member sections 66′ and 68′ of the section 14 may be replaced with substantially rectangular imaging tops or sections 100 and 101′ respectively. Each of the sections 100 and 101′ having elongate slots 101 formed therein to allow for attachment of the hinge mechanisms 70 and 72 in a manner identical or substantially similar to what has been described herein with respect to the frame sections 12 and 14.

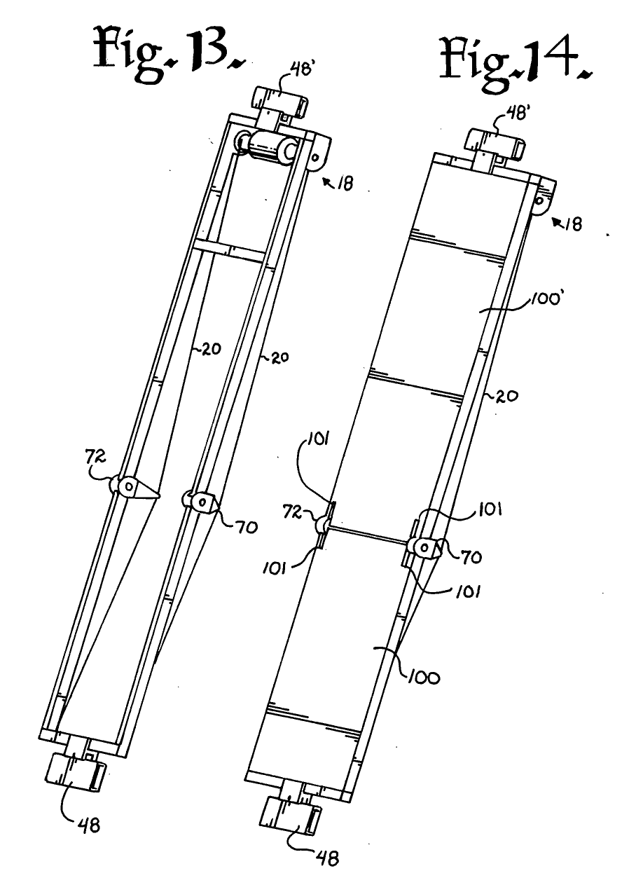

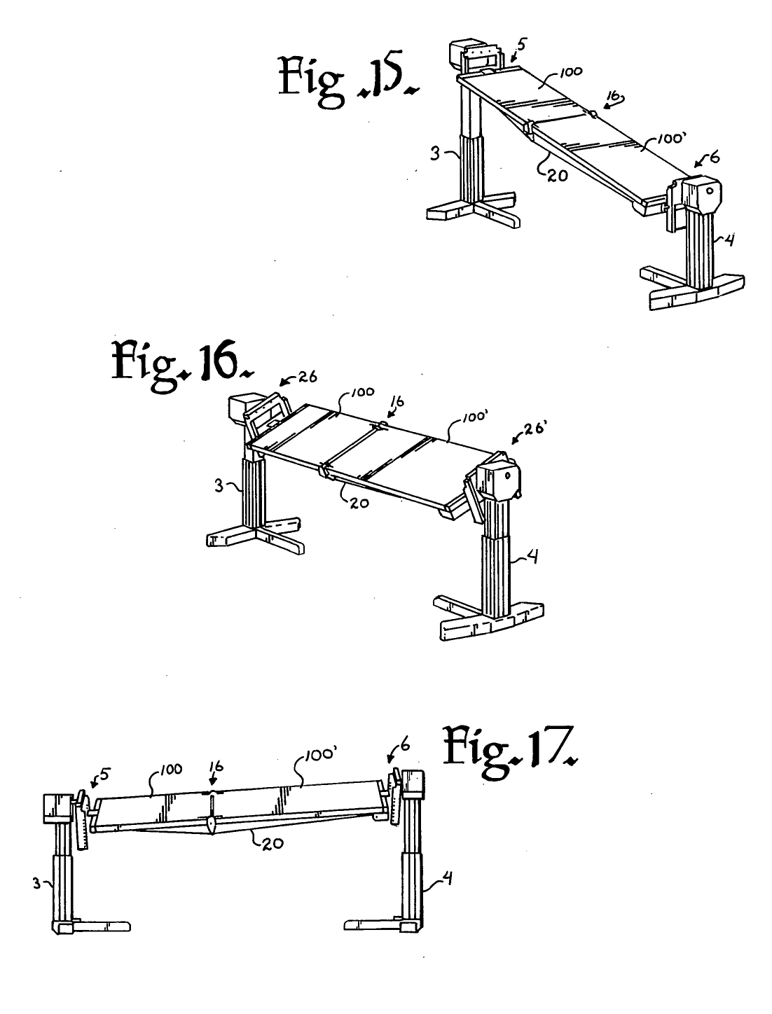

With reference to FIGS. 15-17, the imaging sections 100 and 100′ are illustrated, replacing the frame sections 12 and 14 of the embodiment disclosed in FIGS. 1-12. Each of FIGS. 15-17 represent configurations in which the cable drive 18 is tensioned such that the sections 100 and 100′ are kept in a substantially coplanar configuration. FIG. 15 illustrates a configuration in which the column 3 is telescoped upwardly with the frame sections hinging at the support assemblies 5 and 6, resulting in an inclined position or configuration of the entire patient support. In the illustrated embodiment, the section 100 would preferably receive a patient's head. Therefore, FIG. 15 illustrates a reverse Trendelenburg position or orientation. FIG. 16 illustrates the sections 100 and 100′ again in a substantially common plane with both sections being rotated to a tilted position produced by a powered rotation of the sub assemblies 26 and passive rotation of the assembly 26′ with both columns 3 and 4 otherwise holding the sections 100 and 100′ at the same height. FIG. 17 illustrates both tilting due to rotation of the assemblies 26 and 26′ and also a sloping or inclined position with the column 4 being extended vertically. Thus, FIG. 17 illustrates a Trendelenburg position or orientation with both the sections 100 and 100′ remaining in substantially the same plane. It is foreseen that a bearing block assembly at one or both ends of the table provides for some lateral translation to prevent binding of the hinge mechanisms.

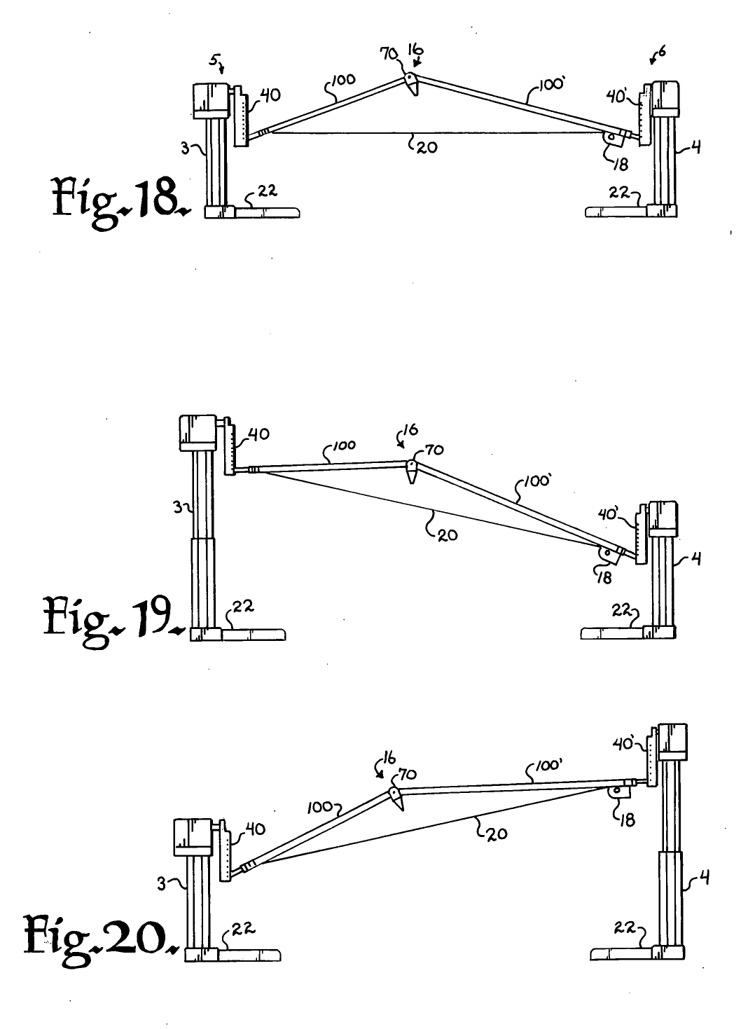

With reference to FIGS. 18-20, there is illustrated three upward breaking or hinging configurations of the structure 1. FIG. 18 illustrates a symmetrical upward breaking configuration wherein the columns 3 and 4 are holding the respective support assemblies 5 and 6 at substantially the same height with the cables 20 being shortened by rotation of the winch motor to result in an upward break or joint in the hinge assembly 16. FIG. 19 illustrates the column 3 being extended to a maximum height and the cables reeled to shorten a distance between the sections 100 and 100′. An example of such an upward break or joint with reverse Trendelenburg would be a head or column 3 height of 43 inches, a foot or column 4 height of 24 inches and a 35 degree upward break with zero degree roll. FIG. 20 illustrates an upward breaking Trendelenburg with the column 4 being extended to a maximum height.

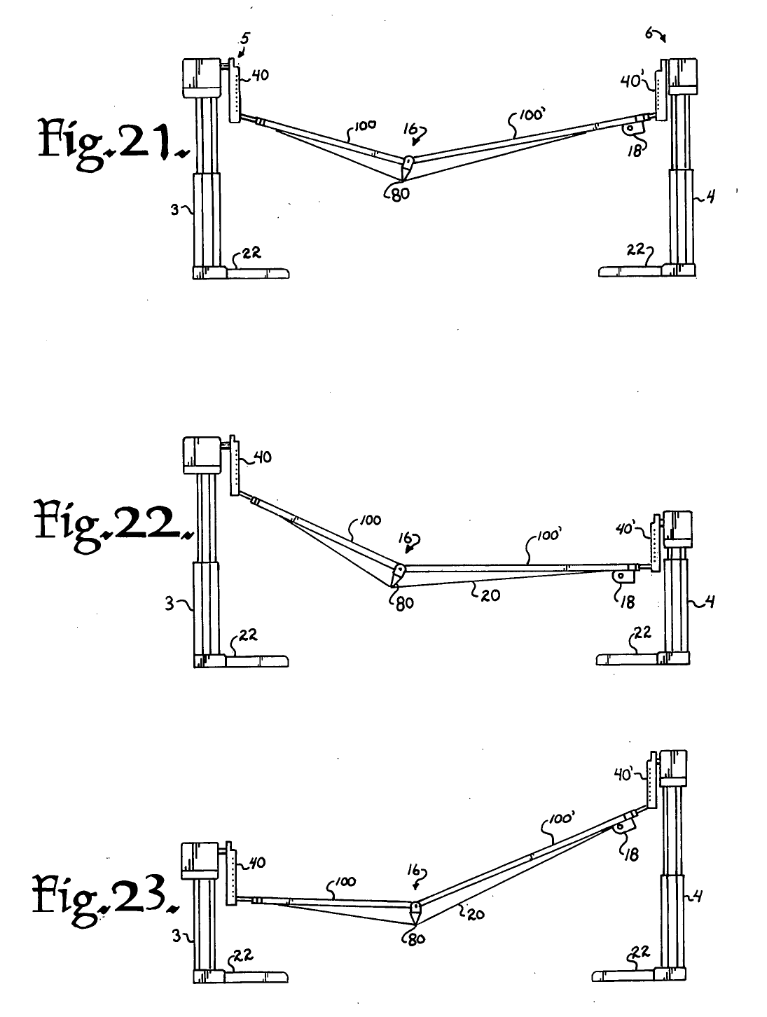

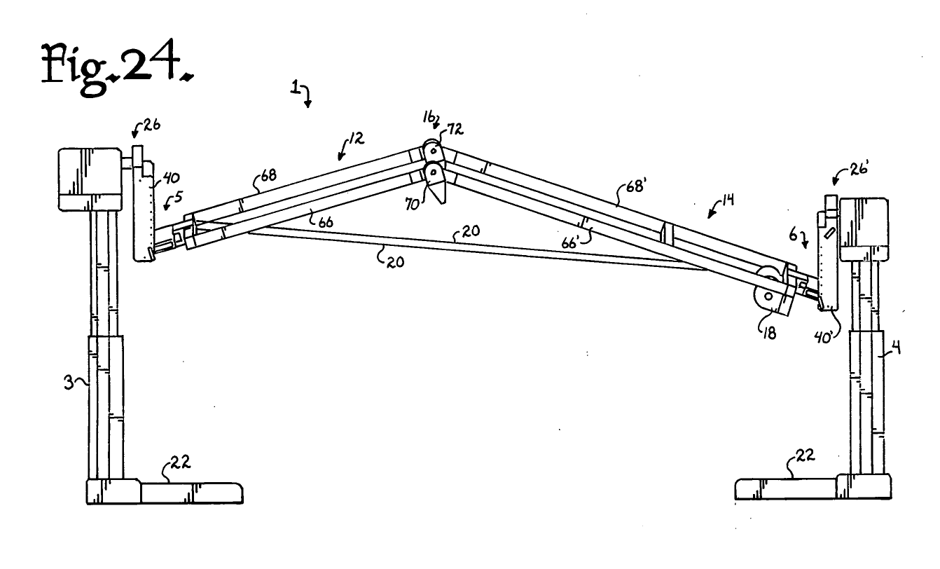

With reference to FIGS. 21-23, there is illustrated three downward breaking configurations of the structure 1. FIG. 21 illustrates a symmetrical downward breaking configuration wherein the columns 3 and 4 are holding the support assemblies 5 and 6 respectively, at the same height with the cables 20 being unwound or slackened to result in a downward break or joint in the hinge assembly 16, the hinges 70 and 72 contacting the cables 20. FIG. 22 illustrates a downward breaking reverse Trendelenburg with the column 3 being extended to a maximum height resulting in a patent's head end being at a maximum height. FIG. 23 illustrates a downward breaking Trendelenburg with the column 4 being extended to a maximum height.

It is noted that in each of the configurations illustrated in FIGS. 18-23, the sub-assemblies 26 may be rotated in either direction, resulting in a tilted or rotated as well as upwardly or downwardly broken or hinged configuration. For example, FIG. 24 illustrates the structure 1 with support frame sections 12 and 14 positioned in a configuration similar to that illustrated in FIG. 19, but also including rotation, resulting in a tilting and upwardly breaking or jointed configuration of the structure 1. An example of the position illustrated in FIG. 24 would be: a head or column 3 height of 41 inches, a foot or column 4 height of 34 inches and a 35 degree upward break or joint with 10 degree roll.

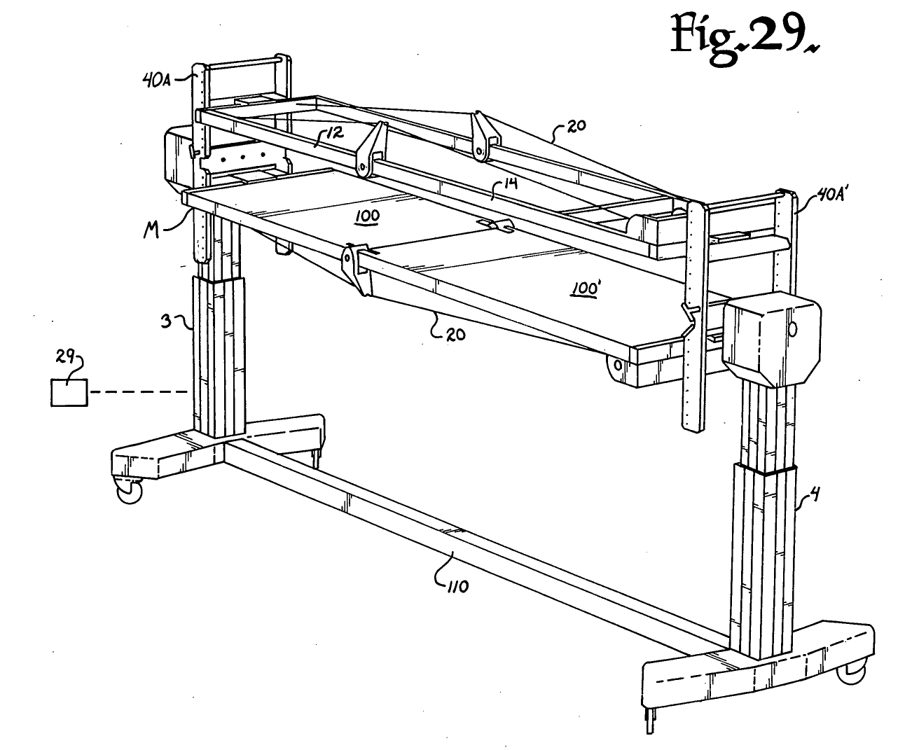

With reference to FIGS. 25-29, another structure, generally 102 according to the invention is illustrated. The structure 102 utilizes all of the elements described herein with respect to the structure 1 and therefore the same references numerals are used for the same elements or features. The structure 102 differs from the structure 1 in that the H-bar posts 40 and 40′ are replaced or modified to be extended H-bar posts 40A and 40A′, allowing for the mounting of two elongate structure 10 and cooperating cable drives 18. In the embodiment shown in FIG. 25, one of the structures 10 includes the frame member 12 and 14 while the other structure is an imaging top having sections 100 and 100′. As previously described herein, the cooperating H-bar posts 40A and 40A′ equipped with a plurality of apertures allows for the placement of the support structures 10 at a variety of locations. For example, FIGS. 25-28 illustrate a first spaced orientation of the elongate frame with respect to the elongate imaging top with the imaging top located at a “lower” position identified by the reference letter L. The identical components are shown in FIG. 29 with the imaging top located at a “mid-position” identified by the reference letter M, illustrating a more compact or closely spaced orientation of the elongate frame with respect to the elongate imaging top than what is shown in FIG. 25.

As illustrated in FIGS. 25-28, the structure 102 provides for the complete rotation and thus a roll-over of a patient by actuation of the motor of the rotation subassembly 26 using the controller 29. The structure 102 shown in FIGS. 25-29 is further illustrated with a non-telescoping base support 110 fixed to each of the columns 3 and 4 and rollers or castors 112 at the base of the structure 102.

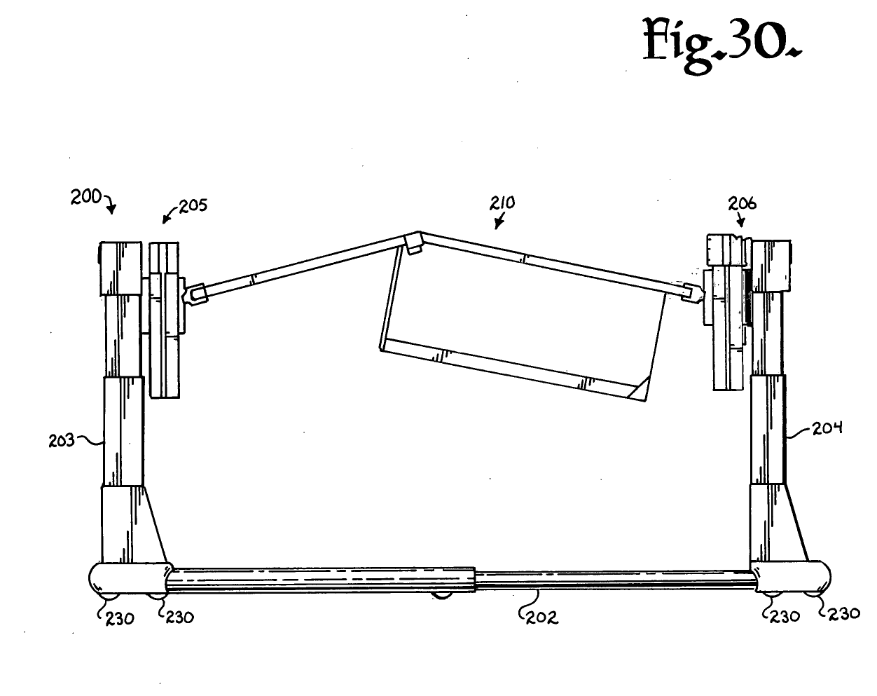

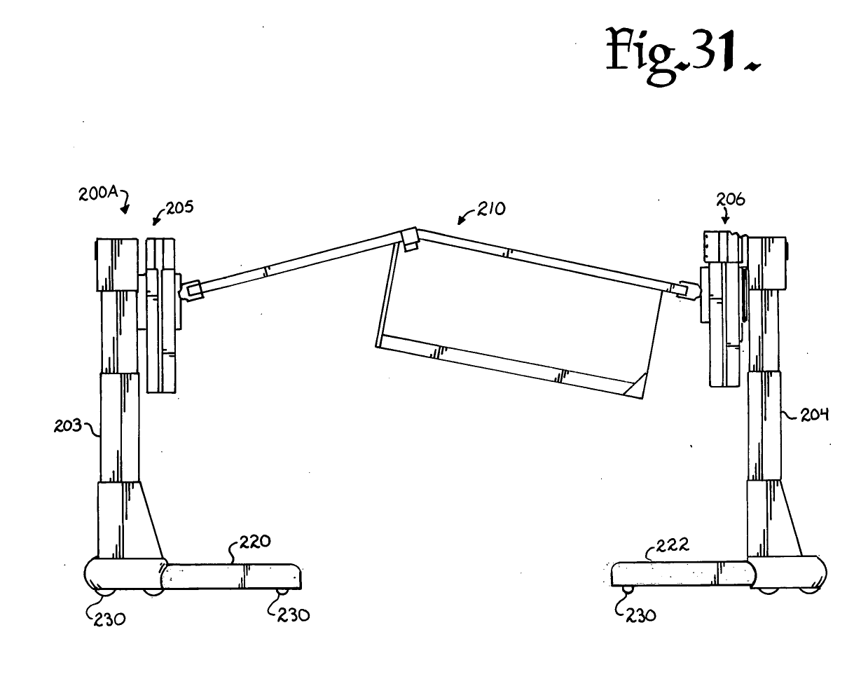

With reference to FIGS. 30 and 31, another embodiment or system according to the invention, generally 200 is illustrated. The system 200 broadly includes an elongate length-adjustable base 202 surmounted at either end by respective first and second upright support piers or columns 203 and 204 which are connected to respective first and second support assemblies, generally 205 and 206. Between them, the support assemblies 205 and 206 uphold an elongated breaking, hingeable or pivotable patient support structure, generally 210. The hinge structure is described in detail in Applicants's U.S. Pat. No. 7,152,261 and also U.S. patent application Ser. No. 11/159,494, both disclosures of which are incorporated by reference herein. The embodiment 200A illustrated in FIG. 31 differs from the structure 200 only in that the length-adjustable base 202 is replaced by a first base 220 attached to the pier 203 and a second base 222 attached to the pier 204. All of the bases 202, 220 and 222 include castors or rollers 230 or some other movable structure to allow the piers 203 and 204 to move toward and away from one another during upward or downward breaking of the structure 210.

It is foreseen that cable drives as described herein, other types of motor drives including screw drives, universal joints, hydraulic systems, robotic mechanisms, electric servo motors, and the like, may be utilized to facilitate both upward and downward breaking of the support structure 210.

Another patient support structure according to the invention, generally 301, is illustrated in FIGS. 32-34. The structure 301 generally includes a horizontally telescoping floor mounted base 302, a conventional or standard telescoping and inclinable operating table support structure 304, a telescoping end support or pier 306 and a hinged or pivotally upwardly and downwardly breaking patient support structure 310 connected to both the structure 304 and the pier 306. The structure 310 is typically configured as an open frame with a head and foot end section. The patient support structure 310 further includes a first cantilevered section 312 and a second section 314 that is passively moved. The first section 312 is fixed to and extends from the operating table support 304 and can be slidably driven toward and away from pier 306. The second section is attached to the pier 306 by a hinge or pivoting assembly 320, such as the support assembly 5 described herein with respect to the structure 1. The hinge mechanism 316 disposed between the support sections 312 and 314 may be a conventional hinge, pivot, or pivot or hinge systems previously described herein. The hinge mechanism is typically configured as a pair of spaced apart hinges.

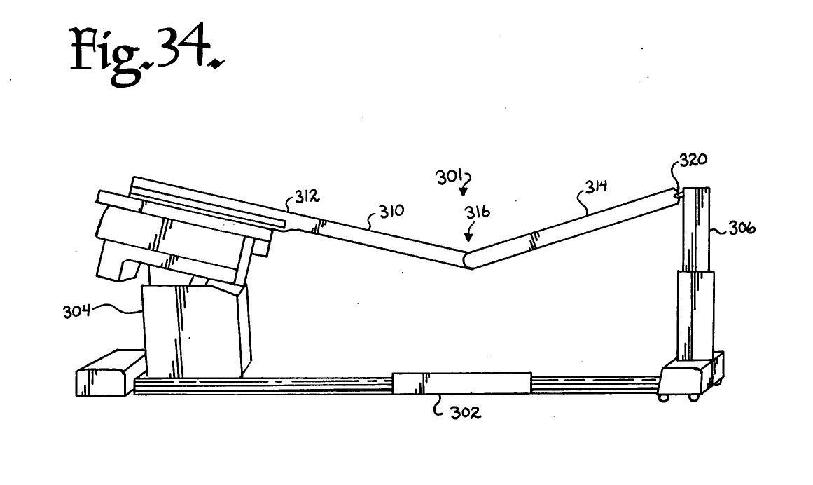

In use, the operating table support 304 utilizes electric or other power means to move the support section 312 up and down and at an incline, as is known in the art. The operating table support 304 can also tilt or rotate from side to side. In response to the movement of the section 312, the section 314 also moves, resulting in upward and downward breaking illustrated in FIGS. 33 and 34, respectively. In some embodiments, in response to the movement of the section 312, the electric powered telescoping base 302 moves the pier 306 toward or away from the support 304. The pier 306 includes a motor for raising and lowering the pier at the connection 320.

As stated above with respect to other embodiments of the invention described herein, it is foreseen that cable drives as described herein, other types of drives including screw drives, hydraulic systems, and the like, may be utilized to facilitate both upward and downward breaking of the support structure 310 at the joint 316.

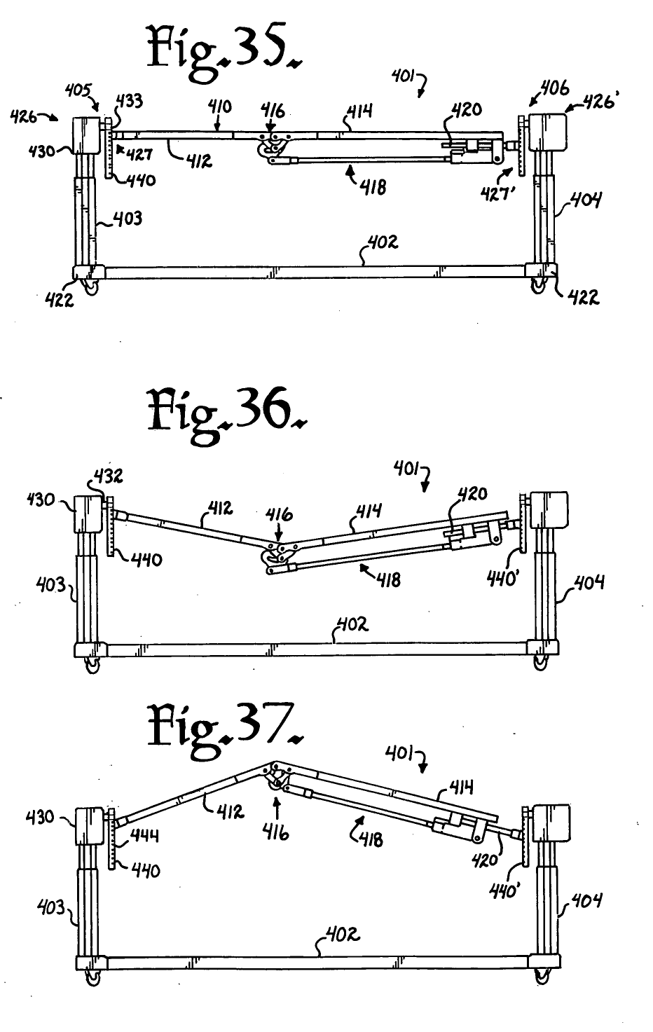

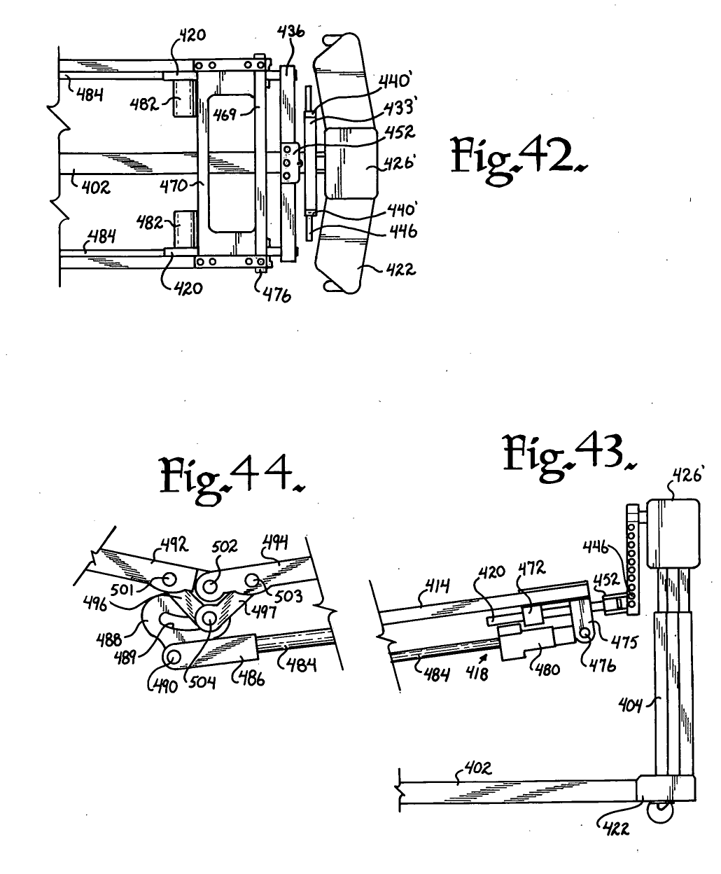

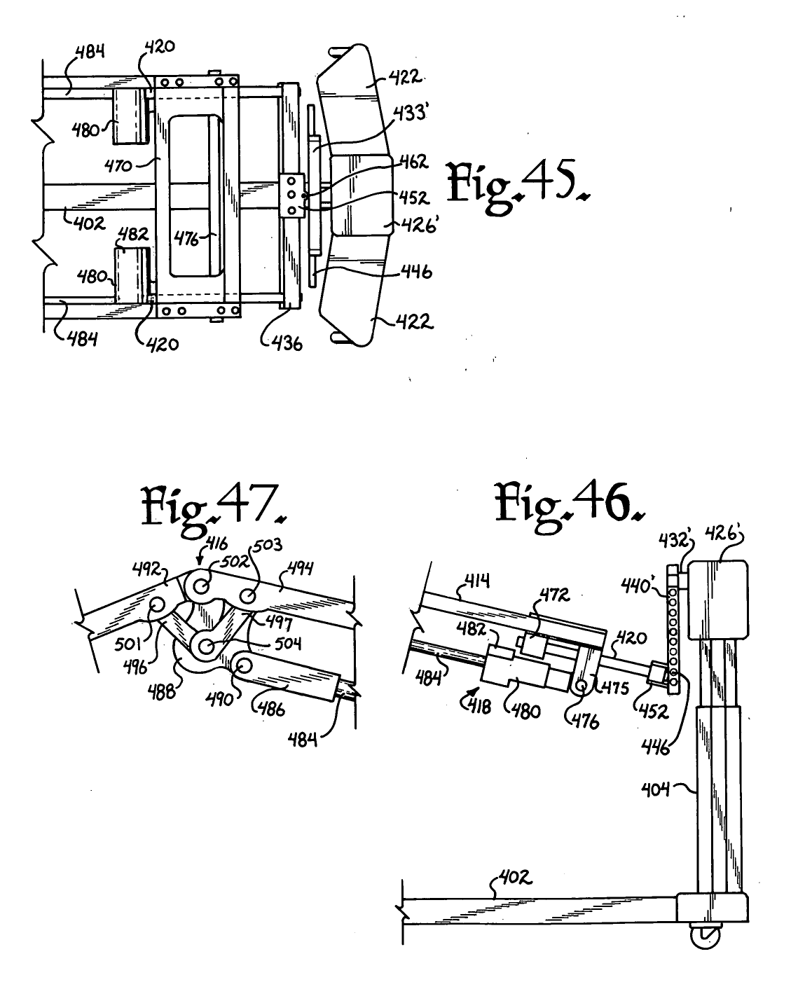

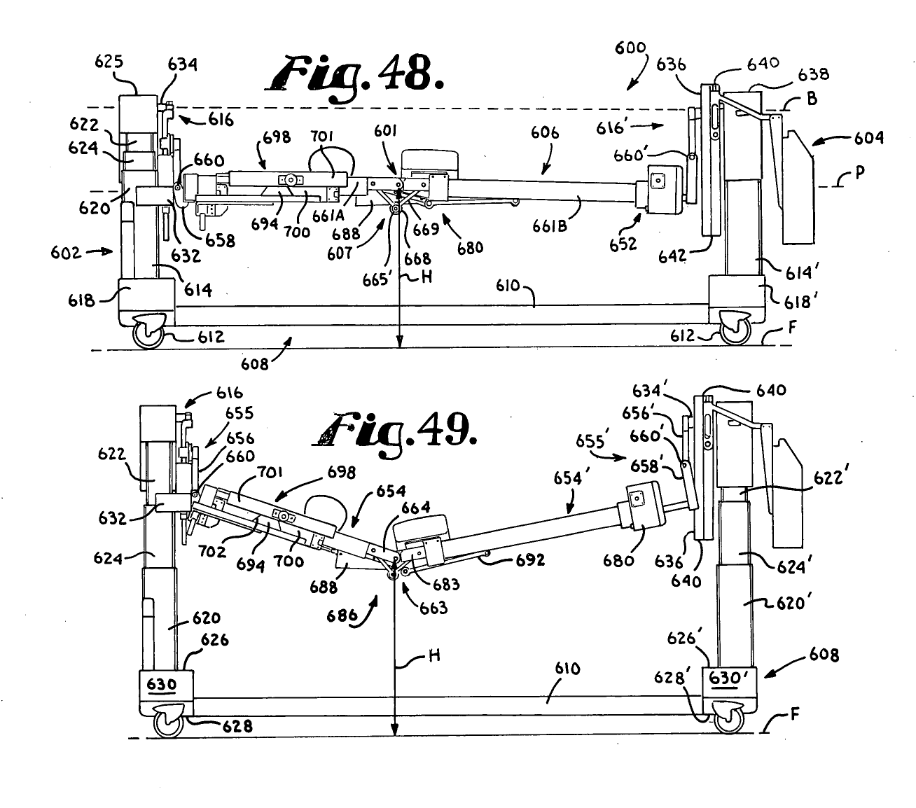

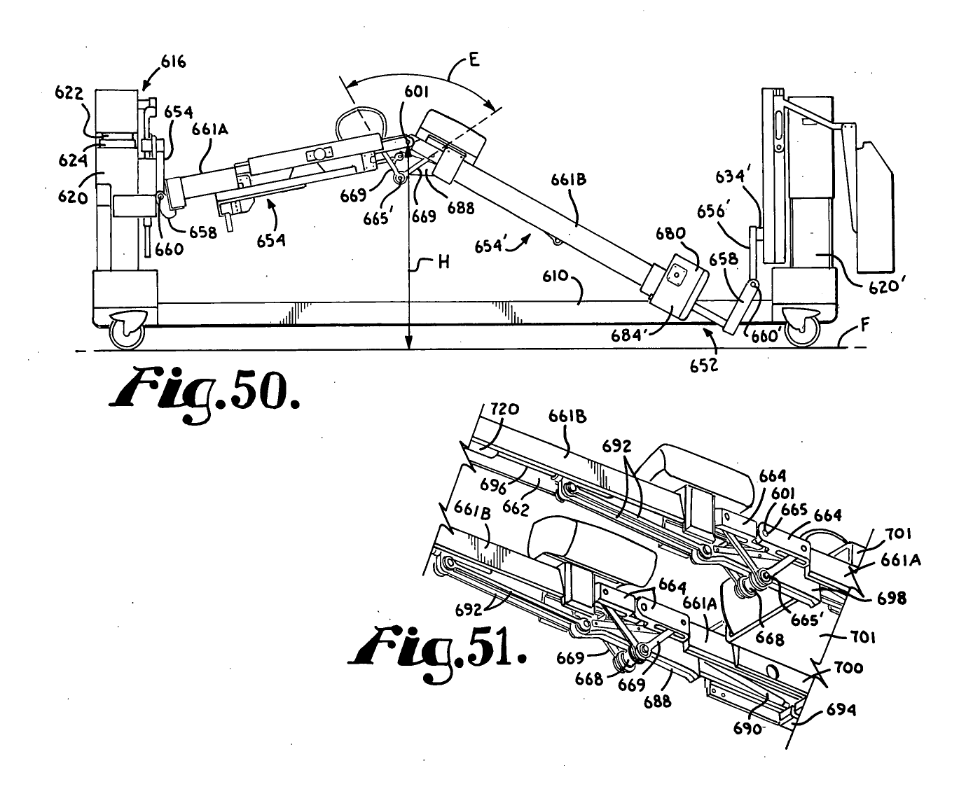

With reference to FIGS. 35-47, another patient support structure according to the invention, generally 401 includes first and second upright support piers or columns 403 and 404 that are connected to one another by a non-telescoping base support 402. In some embodiments according to the invention, each column may be surmounted on an independent movable or stationary base. The column 403 is connected to a first support assembly, generally 405 and the column 404 is connected to a second support assembly, generally 406. Between them, the support assemblies 405 and 406 uphold at least one removable elongate and articulate, substantially centrally jointed or breaking patent holding or support structure, generally 410. The assembly includes a first frame section 412, a second frame section 414 and a pair of identical hinge assemblies, generally 416, disposed between and connecting the first and second frame sections 412 and 414. In the illustrated embodiment, the first frame section 412 for holding a head and upper body of a patient is of a slightly shorter longitudinal length (along an axis X) than the second frame section 414. Therefore, the spaced hinge assemblies 416 are approximately centrally located relative to a body of a patient being placed on the structure 410. In the illustrated embodiment, the hinge assembly further includes a drive system that includes a pull rod assembly, generally 418, and cooperating spaced slider bars 420. Again, other drive systems are foreseen.

The columns 403 and 404 are substantially similar in form and function to the columns 3 and 4 previously described herein with respect to the structure 1. The columns 403 and 404 are supported by outwardly extending feet 422 that include casters that may be equipped with a floor-lock foot lever for lowering the feet 422 into a floor-engaging position. The columns 403 and 404 each include two or more telescoping lift arm segments respectively that permit the height of each of the columns 403 and 404 to be selectively increased and decreased in order to raise and lower all or a selected portion of the connected patient support structure 410.

Each of the support assemblies 405 and 406 generally includes a rotation subassembly 426 and 426′ and an angulation subassembly 427 and 427′, respectively, that are the same or substantially similar to the subassemblies 26, 26′, 27 and 27′ previously described herein with respect to the structure 1. In the illustrated embodiment, the angulation subassembly 427 connected to the frame 412 for holding the head and upper body of a patient is shown as substantially identical to the subassembly 27 and therefore shall not be described further herein. The subassembly 427′ is substantially similar to the subassembly 27′, but with some modifications, including a frame 436 disposed transverse to the overall longitudinal axis X of the structure 401, the frame 436 providing for slidable support of the pair of identical slider bars 420 that are disposed at either side of the frame 414 and near the subassembly 427′.

Similar to the rotation subassembly 26 previously described herein, the rotation subassembly or mechanism 426, includes at least one motor housing 430 surmounting the support column 403. It is foreseen that a cooperating motor may also be mounted on the support column 404. A main rotational shaft 432 extends from the motor housing 430 that turns a rotation structure or bar that in turn is connected to and rotates the patient support 410 about a longitudinal axis. In particular, the motor housing 430 contains a rotary electric motor or other actuator drivingly engaged with the shaft 432. The rotation mechanism 426 is operated by actuating the motor using a switch or other similar means. The shaft 432 rotationally cooperates with a pair of substantially vertically disposed translation posts or H-bar posts 440, the posts 440 being attached to and disposed at either end of the transverse rotation structure or bar 433. Each H-bar post 440 includes a plurality of apertures 444, allowing for selective, hinged vertical placement of the frame section 412 identical or substantially similar to what has been described previously herein with respect to the H-bar posts 40, the angulation sub-assembly 27 and the frame end section 58 of the frame section 12 previously described herein with respect to the structure 1.

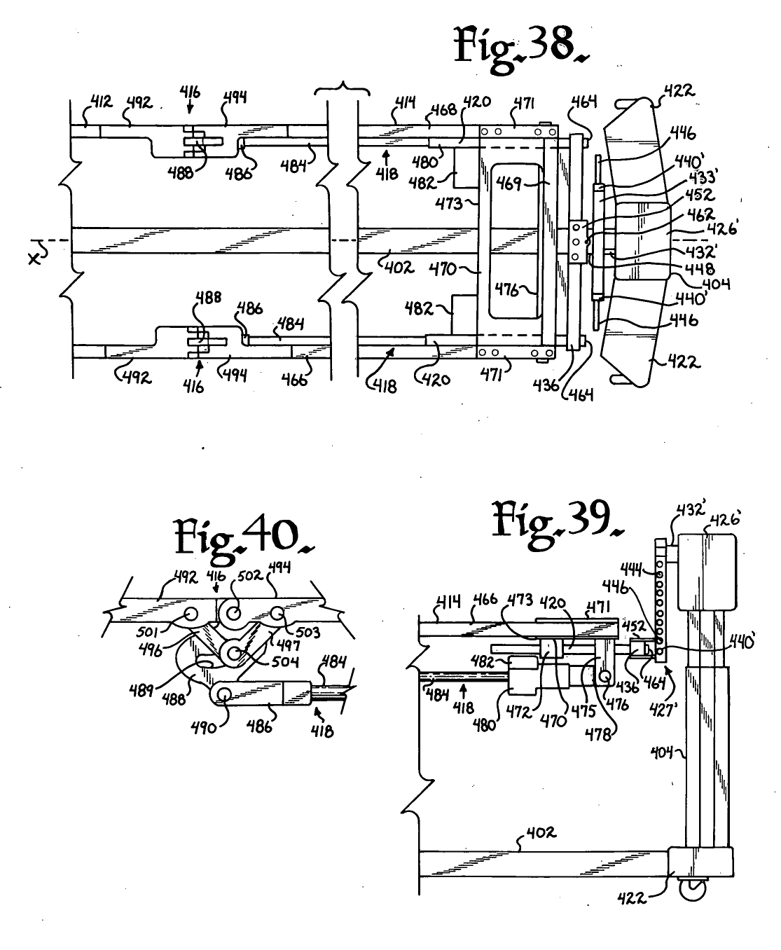

With particular reference to FIGS. 38-40, as stated above, the sub-assembly 426′ is substantially similar to the sub-assembly 426 and therefore may include a motor and further includes either an active or passive rotational shaft 432′ that engages a rotation structure or bar 433′ that is attached to a pair of substantially vertically disposed H-bar posts 440′. A plurality of cooperating apertures 444′ formed in the posts 440′ provide passageway for a pivot pin 446 to extend therethrough. The pivot pin 446 is receivable in each cooperating pair of apertures 444′, allowing for selective placement of a translation connector 448 that is sized and shaped to be received between the pair of posts 440′ and also receive the pivot pin 446 therethrough. The pin 446 and connector 448 are thus positionable in an orientation transverse to the longitudinal axis X of the patient support frame 410 at a variety of heights to be selected by the surgeon and readily changeable, even during surgery if necessary, to vary the height of the frame section 414. The multiple location or height feature is also advantageous when more than one frame or patent structure is mounted in tandem, for example, when both a frame and imaging table are used together, such as is shown in the embodiment illustrated in FIGS. 25-29. The position of the frame or other structure may be desirably changed to provide close proximity to an imaging top with a distance between a patient support and an imaging top being expandable or reduceable depending upon the size or other attributes of a patient and surgical or other requirements. The connector 448 has a slot for receiving the pivot pin 446. It is noted that the H-bar support 440′, apertures 444′, elongate transverse pin 446 and translation connector 448 are the same or substantially similar in form and function with the respective support 40, apertures 44, transverse pin 46 and translation connector 48 previously described herein with respect to the structure 1.

The translation connector 448 is in turn attached to a pivot connector 452 that is substantially similar to the pivot connector 52 previously described herein with the exception that rather than being attached directly to an end piece or section of the patient support frame 414, the pivot connector 452 is fixed to the frame 436 that is fixed to and supports the slider bars 420 near end surfaces 464 thereof. Thus, the slider bars 420 are in a hinged relationship with the H-bar supports 440′. The slider bars 420 are also in slidable attachment with the frame section 414 and disposed substantially parallel to a longitudinal axis of the section 414 as will be described in greater detail below. Such slidable attachment facilitates upward and downward breaking or hinging of the section 414 with respect to the section 412 at the hinge mechanism 416. Also as more fully described below, the pull rod assembly 418, that is connected to both the frame section 414 and the hinge mechanism 416, is extendable and retractable, controlling the hinge or break angle of the patient support 410 and rendering the support 410 rigid at a desired upward or downward break or joint of the hinge mechanism 416.

With particular reference to FIGS. 38 and 39, the support frame section 414 includes opposed elongate and parallel frame sections 466 and 468 attached to one another by a transverse end frame section 469. A support plate 470 is attached to and is disposed below each of the sections 466, 468 and 469 to provide additional support and stability to the frame section 414 at and near the end section 469. Further support is provided by a pair of frame support plates 471, both of which are fixed to the end support frame section 469 near one end thereof; one plate 471 being fixed to the section 466 and the other plate 471 being fixed to the section 468. At least one pair of slider bar holding structures 472 are fixed to the support plate 470 and extend downwardly therefrom at each of the frame sections 466 and 468. Each structure 472 includes a through bore that extends parallel to the frame sections 466 and 468, the structure 472 for slidably receiving one of the slider bars 420 directly below one of the frame sections 466 and 468 and also orienting the pair of slider bars 420 in a direction substantially parallel to the frame sections 466 and 468. The illustrated slider bar holding structures 472 are spaced from the end frame section 469 and located near a forward edge 473 of the plate 470. In the illustrated embodiment, the holding structures 472 are also bolted to the frame sections 466 or 468. A pair of pull-rod supports 475 are also fixed to the support plate 470 and the frame 414 and extend downwardly therefrom at each of the frame sections 466 and 468 and also downwardly from the end frame section 469. Each structure 475 includes a through bore for receiving a transverse pivot pin or bar 476 mounted below the slider bars 420. The pull-rod assembly 418 is attached to the support 475 at the pivot pin 476 and is thus in hinged relationship with the support 475, pivotally attached thereto at end portions 478.

The pull-rod assembly 418 further includes a pair of housings 480, each housing attached to an end portion 478 and having a powered actuator 482 cooperating with one of a pair of rotatable extendible and retractable rods 484 and a pair of hinge connectors 486, each pivotally attached to a respective cam plate 488 of the respective hinge mechanism 416 at a respective pivot pin 490. The cam plate 488 has a substantially centrally located curvilinear wall 489 forming a curvate aperture or slot, a lower circular aperture for receiving the pin 490 and an upper circular aperture for receiving a pin 502, described in greater detail below. Each pull rod 484 is rotatably mounted within one of the housings 480, such rotation being controlled by operation of the actuator 482 located in the housing 480 and engaged with the rod 484 to screw and thus selectively move or draw the rod 484 into or away from the hinge mechanism 416 in a direction along a longitudinal axis of the rod 484, that in turn results in breaking or jointing of the patient support 410 at the hinge mechanism 416. It is foreseen that other embodiments according to the invention may utilize other types of push/pull rods or mechanisms, including, for example hydraulic systems. An additional centrally located pull-rod or piston may be included to provide additional support. Furthermore, other hinge mechanisms according to the invention may be utilized in lieu of the mechanism 416, for example including, but not limited to, polyaxial joints, roller with spokes, sprockets, toothed gears, universal axis gears, or the like.

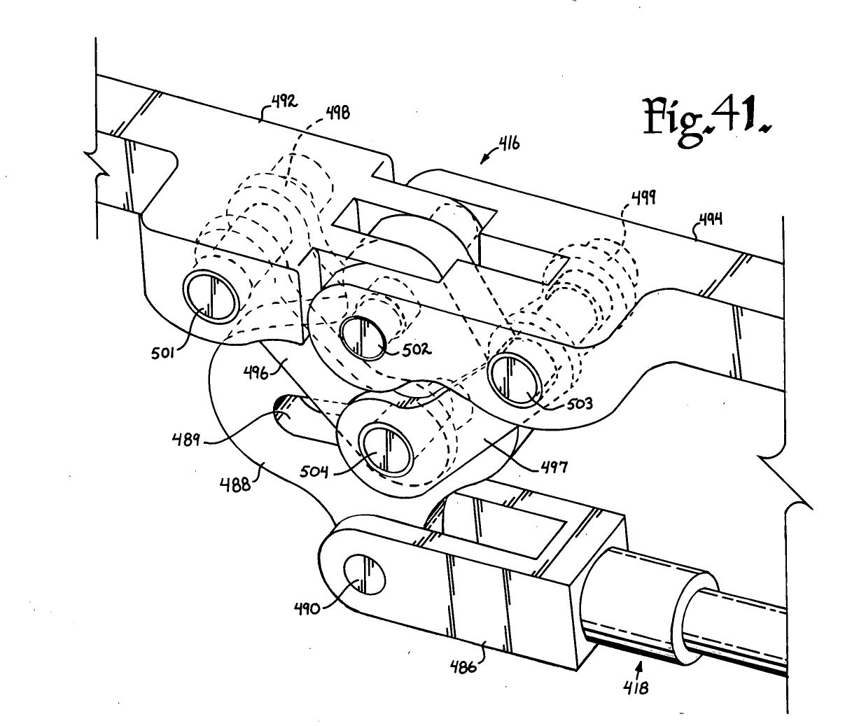

With particular reference to FIG. 41, the illustrated pair of hinge mechanisms 416, each having a cam plate 488, further include a pair of forked arms 492 extending from the frame section 412 and a pair of cooperating forked arms 494 attached to and extending from the section 414. Hinge arms 496, 497, 498 and 499 having apertures near opposite ends thereof for receiving pivot pins cooperate with the respective cam plate 488 and adjacent forked arms 492 and 494 at pivot pins 501, 502, 503 and 504. All of the pivot pins 490, 501, 502, 503 and 504 are disposed transverse to the longitudinal axis X of the patient support structure 401. In particular, the pivot pin 501 is received by circular apertures located near first ends of the hinge arms 496 and 498 and a circular aperture in the arm 492, thus pivotally attaching the arm 492 with both the hinge arms 496 and 498. The pivot pin 502 is received by an upper circular aperture in the cam plate 488 and circular apertures located near the ends of each of the forked arms 492 and 494, thus pivotally attaching the cam plate 488 with both of the forked arms 492 and 494. The pivot pin 503 is received by circular apertures located near first ends of the hinge arms 497 and 499 and a circular aperture in the arm 494, thus pivotally attaching the arm 494 with both the hinge arms 497 and 499. The pivot pin 504 is received by the slot 489 and also by circular apertures located near second ends of the hinge arms 496, 497, 498 and 499, thus pivotally attaching all four hinge arms 496, 497, 498 and 499 with the cam plate 488 at the slot 489.

Also, with particular reference to FIGS. 35 and 38-41, the structure 401 is shown in a neutral, planar orientation, with the pull-rod assembly 418 holding the hinge mechanism 416 in such neutral position, with the forked arms 492 and 494 in parallel. In such position, the pin 504 is located at or near a rear-ward end of the slot 489.

With reference to FIGS. 42-44, as the rod 484 is rotated to selectively lengthen the rod 484, the pin 504 remains near the rear-ward end of the slot 489 and the pushing of the rod toward the hinge mechanism 416 pivots the cam plate 488 at the pivot pin 490, causing the arms 492 and 494 to move toward the rod hinge connector 486 and thus pivot the patient support at the pin 502, causing a downward break or joint in the patient support 410. With reference to FIGS. 45-47, as the rod 484 is rotated to selectively shorten the length thereof, the support portion 414 slides along the slider bars 420 away from the end support 404. At the same time, the pin 504 slides along the slot 489 to an opposite or forward end thereof as the cam plate pivots in a forward direction about the pin 490. The movement of the rod 484 thus causes an upward break at the pivot pin 502. In the illustrated embodiment, the patient frame is pinned at the head end, but is free to move along the fixed slider bar 420 at the foot end, providing dynamic support to the patient frame. The slider bar mechanism can be attached to a bearing block mechanism to provide lateral translation movement, as described previously.

It is noted that since the patient frame is free to move over the slider bar, a horizontal force component is generated by the combined components of the patient support. When the support is broken or jointed upward, the angle of the foot end frame imparts a horizontal force on the slider that urges the end supports 403 and 404 toward one another. When the table is broken downward, a horizontal force develops that tends to push the end supports apart. It has been found that the magnitude of the horizontal force is a function of support loading and break angle, and thus, for example, if a working limit of five hundred pounds is selected for the patient support, a worst case of horizontal loading is only about fifty-eight pounds at an upward break or joint of thirty-five degrees. It is noted that the illustrated structure 401 advantageously supports a breaking or jointing range from about thirty-five degrees up to about twenty degrees down. Throughout such range, the horizontal forces imposed by the structure are minimized by the illustrated locked support frame that moves on a slider bar at the foot end of the support.

As with the structure 1 configurations illustrated in FIGS. 18-23, the upward and downward breaking of the patient support 410 may be modified by placing the portions 412 and 414 at different vertical locations along the H-bar supports 440 and 440′, thus resulting in symmetrical or asymmetrical breaking configurations. Furthermore, the portions 412 and 414 may be rotated or tilted as described above with respect to the structure 1.

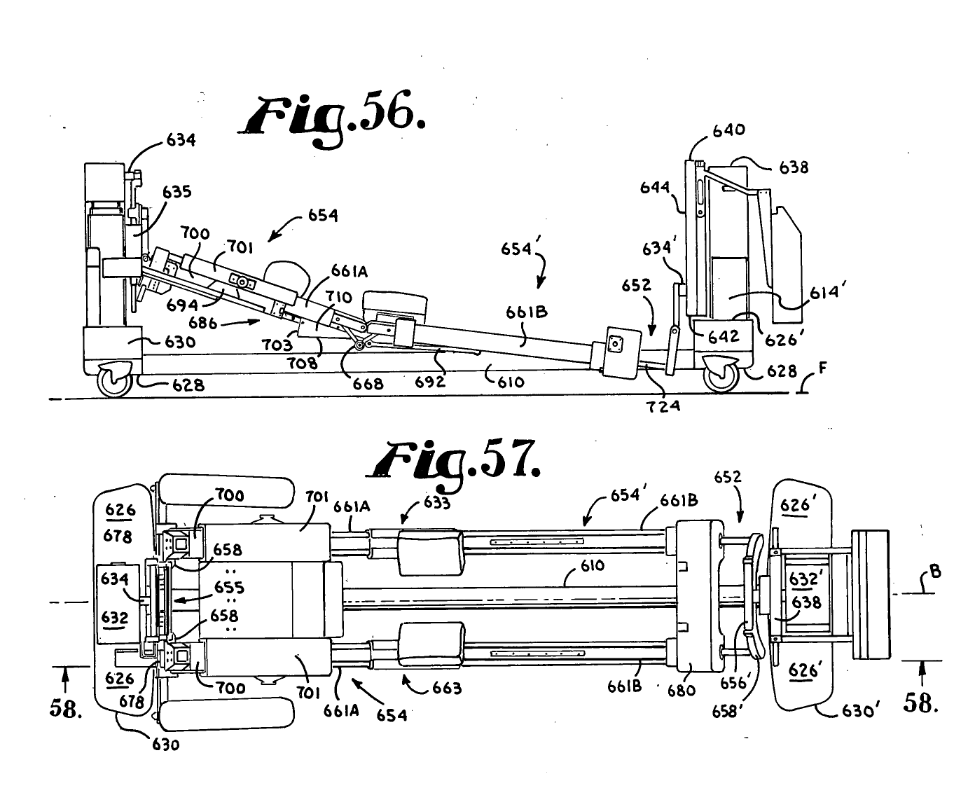

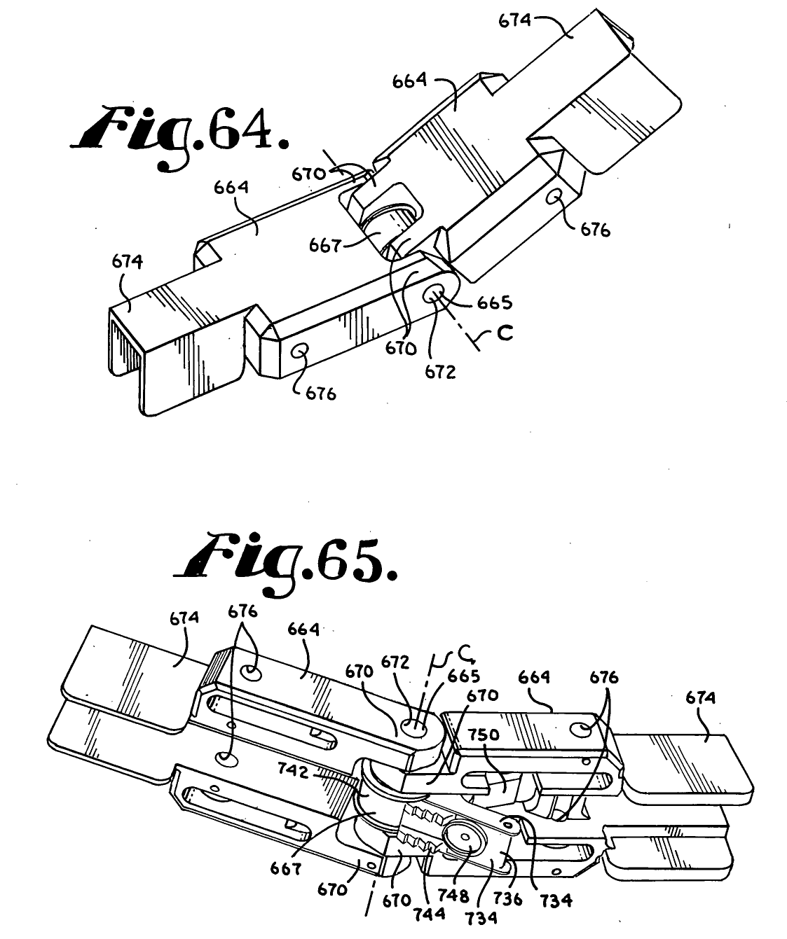

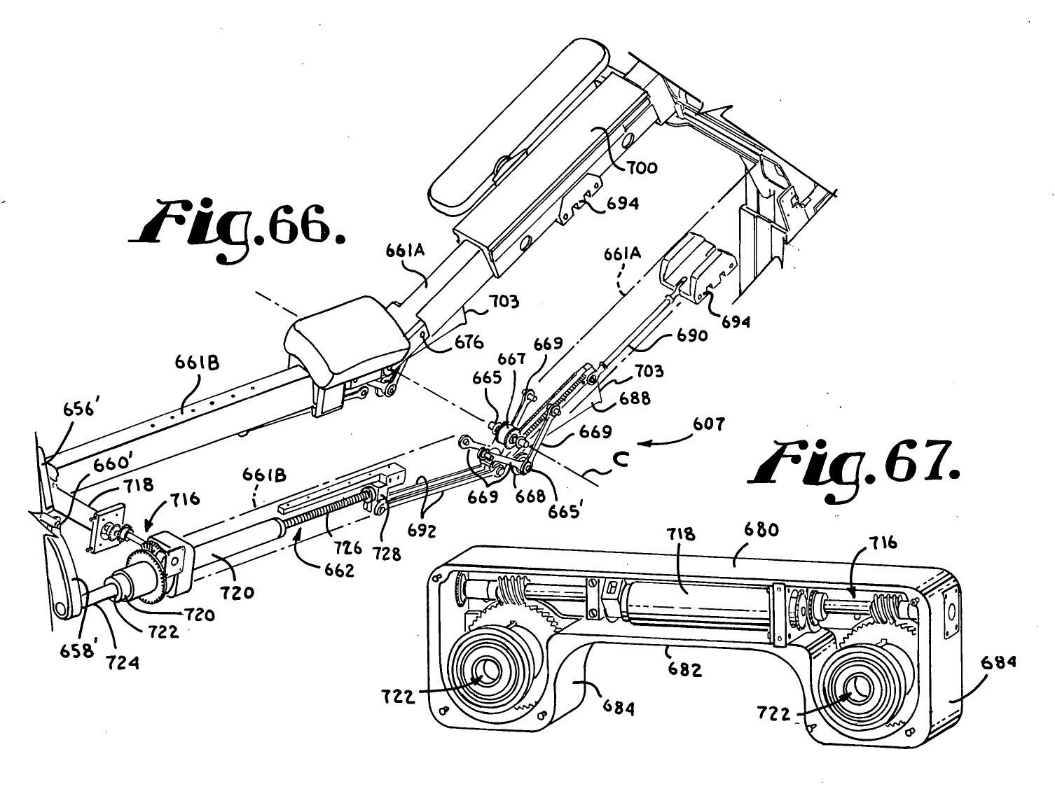

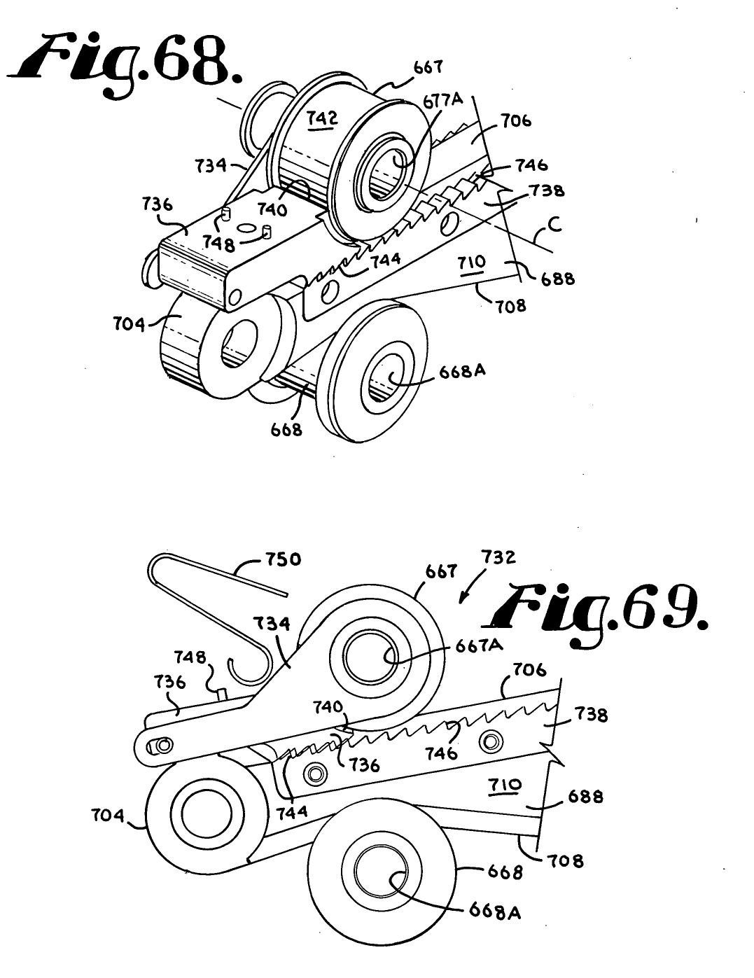

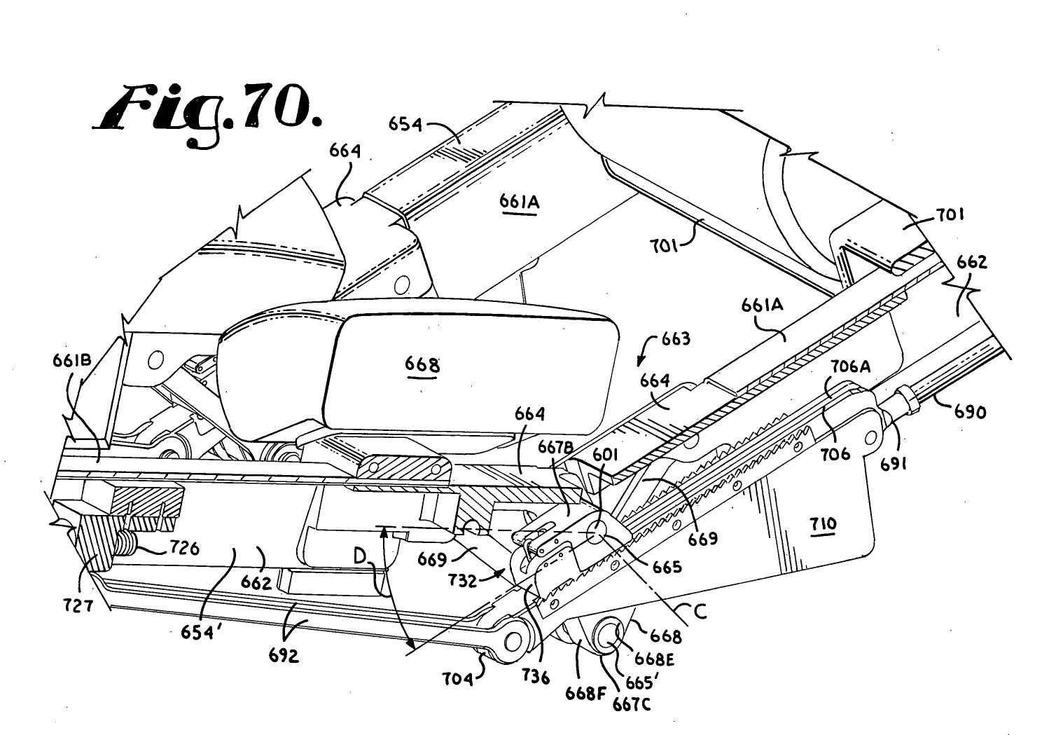

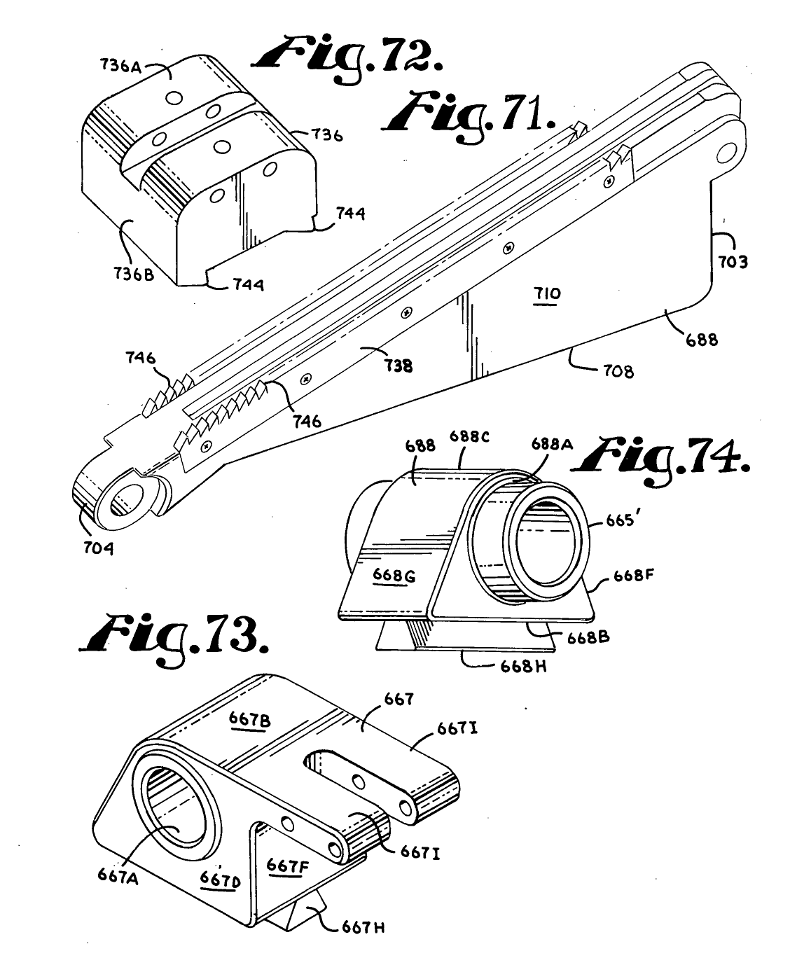

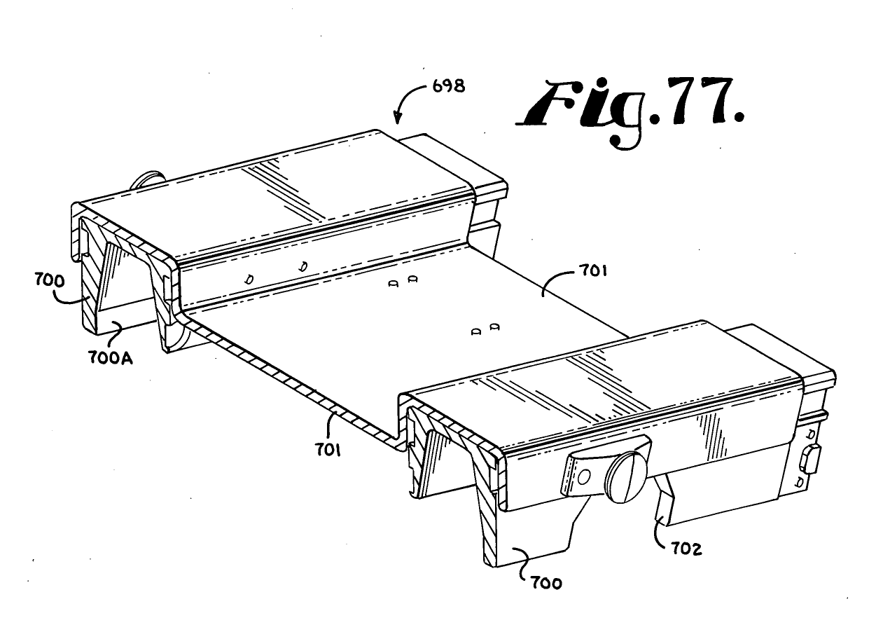

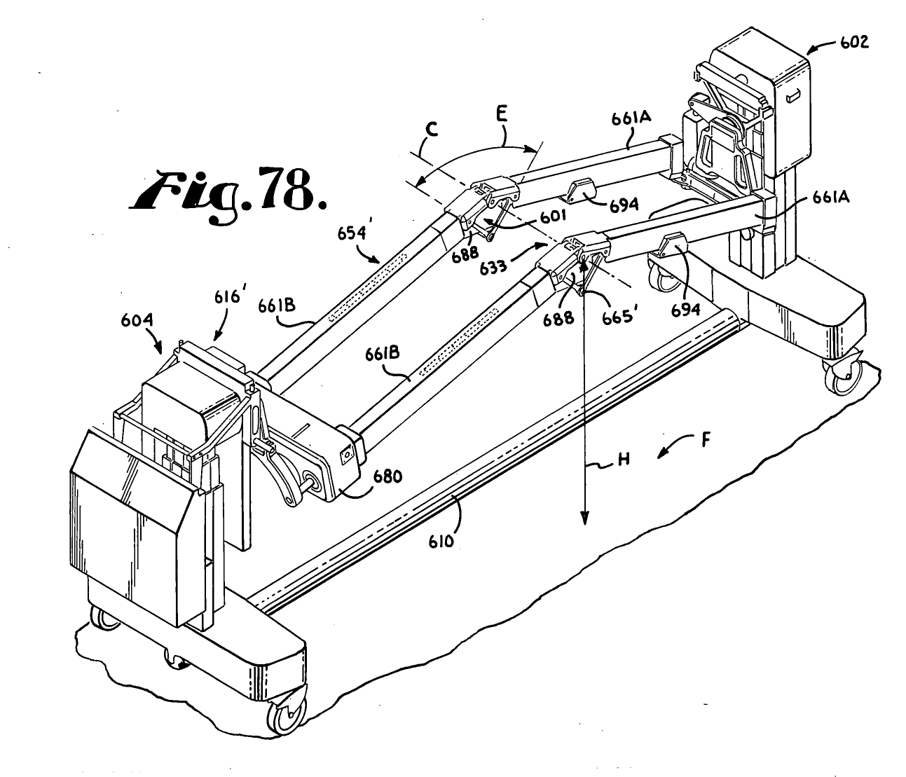

FIG. 48 through FIG. 78 illustrate a non-incrementally, continuously or infinitely adjustable patient support and articulation system or apparatus, generally 600, for supporting a prone patient during a medical procedure, and to modify or change the angle of articulation of the patient, such as at a point of articulation, generally 601, so as to selectively articulate, flex or extend the patient's spine, preferably without substantially changing a height H of the point of articulation 601 relative to a floor F supporting the apparatus 600 according to the invention during a particular surgery. However, the height H of the point of articulation 601 is also variable, for example to adjust for the height of different surgeons or for particular procedures. The apparatus 600 includes a longitudinal axis of rotation B (see FIGS. 48 and 57), a perpendicular axis of rotation C associated with the point of articulation 601 (see FIGS. 64-66, 68, 70 and 78), spaced head-end and foot-end lift subassemblies, generally 602 and 604, also referred to as first and second piers or columns, a patient support subassembly, generally 606, an articulation subassembly, generally 607, and a powered actuator. As discussed below, the axis B may also be referred to as a roll axis, and the axis C may also be referred to as a pitch axis. The axis C runs both substantially perpendicular to the axis B and substantially parallel with the floor F. The patient support subassembly 606 may also be referred to as a patient support structure. The patient support subassembly 606 is suspended between the head-end and foot-end lift subassemblies 602 and 604, respectively.

The head-end and foot-end lift subassemblies 602 and 604, also referred to as piers or vertical translation subassemblies, are joined by a non-telescoping base support structure, generally 608, which may include a cross-bar 610 running parallel with the axis B and a plurality of casters 612. The base support structure 608 holds the lift subassemblies 602 and 604 in opposed spaced relation to one another, and prevents the lift subassemblies 602 and 604 from toppling over during operation of the apparatus 600 due to the large forces exerted on the apparatus 600 by the weight of a patient supported by the apparatus 600 during surgery. Accordingly, subassemblies 602 and 604 do not move substantially along the longitudinal axis relative to one another or closer together or farther apart during operations of the apparatus 600.

In some circumstances, the head-end and foot-end lift subassemblies 602, 604 and the base support structure 608 are referred to as the “base,” which reversibly connectable, joinable or attachable to and suspends the patient support subassembly 606 above the floor F.

Referring to FIGS. 48-50, 56-60, 62-63, the first or head-end lift subassembly 602 provides for continuous adjustable raising and lowering of the head-end of the patient support subassembly 606 over an infinitely adjustable range of heights and, for example, a distance of from about 0.5-inches or less up to about 6-inches, up to about 1-foot, up to about 1.5-feet, up to about 2.0-feet, up to about 2.5-feet, up to about 3.0 feet or more, in cooperation with other components of the apparatus 600, as described herein. By infinitely adjustable range is meant a range of values with maximum and minimum values, and the property of being infinitely or continuously adjustable between these maximum and minimum values, as opposed to being incrementally adjustable.

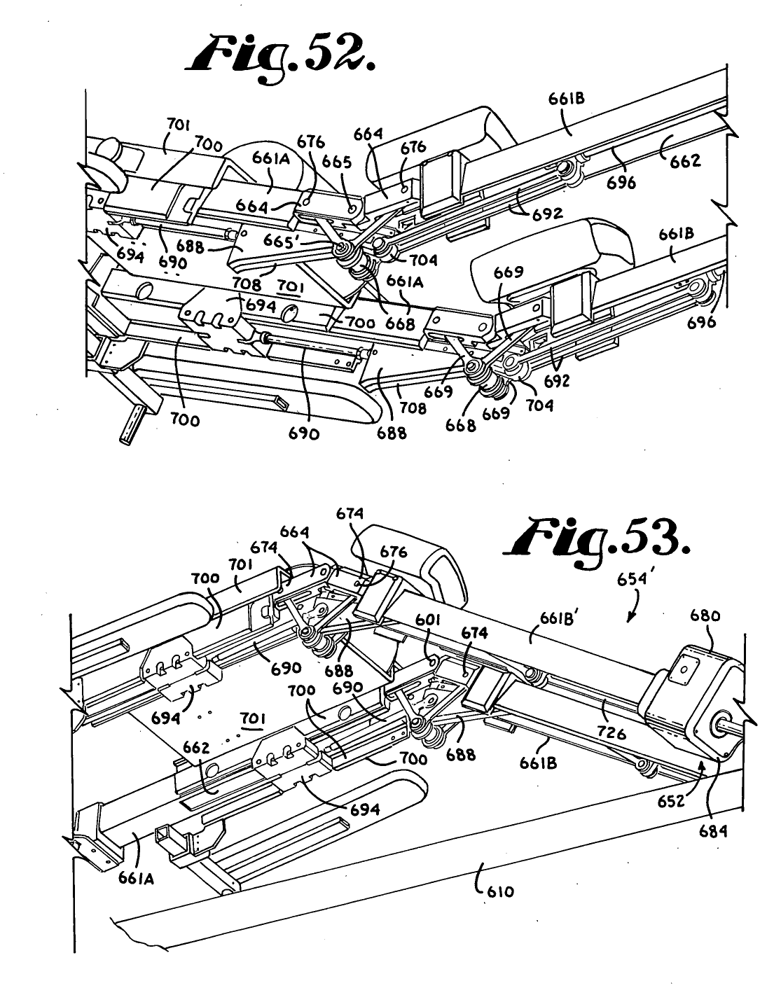

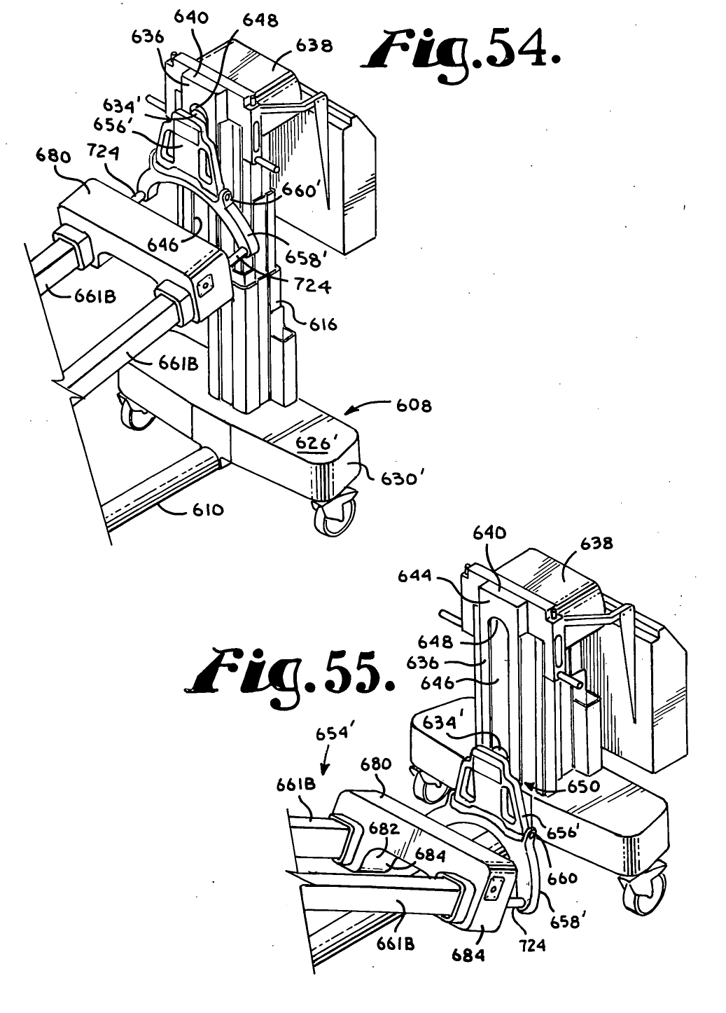

The head-end lift subassembly 602 operates in concert with or cooperates with other apparatus components, such as the foot-end lift subassembly 604 and the articulation subassembly 607, such that an angle of articulation D (see FIGS. 59 and 60) of the articulation point 601 may be modified without a substantial change in height H of the articulation point 601 during a particular surgery, so as to maintain the surgical site of the patient at a preferred height for the surgeon conducting the surgery. In an exemplary embodiment, in order to maintain the point of articulation 601 at a selected height H during upward breaking of the patient support structure 606, such as is shown in FIG. 50, the head-end and foot-end lift subassemblies 602, 604 are adapted to lower the respective outboard ends of the patient support structure 606 an amount, or distance, sufficient to maintain the height H, wherein such vertical translation is coordinated or synchronized with the rotational movement, angulation or articulation at the point of articulation 601. In another exemplary embodiment, in order to maintain the point of articulation 601 at a selected height H during downward breaking of the patient support structure 606, such as is shown in FIG. 49, the head-end and foot-end lift subassemblies 602, 604 are adapted to raise the respective outboard ends of the patient support structure 606 an amount, or distance, sufficient to maintain the height H, wherein such vertical translation is coordinated or synchronized with the rotational movement, angulation or articulation at the point of articulation 601.

The head-end lift subassembly 602 also provides for continuously adjustable, non-segmented rotation or tilting of the patient support subassembly 606 in, or within, an infinitely adjustable range from about 0° to about 90° in either direction, and for example, about ±5°, ±10°, ±15°, ±20°, ±25° or more relative to the axis of rotation B, also in cooperation with the other components of the apparatus 600, as described herein. The head-end lift subassembly 602 includes an individually operable and continuously adjustable first or primary elevator 614, or primary lift subassembly or vertical translation subassembly, a rotational or roll subassembly, generally 616, and a footing 618, which are described in greater detail below.

The primary elevator 614, of the head-end lift subassembly 602, is a vertical translator that actively moves the head-end, or head outboard end, of the patient support subassembly 606 upward and downward. In the illustrated embodiment, the primary elevator 614 includes at least two risers, such as a lower riser 620 and an upper riser 622, and an internal motorized structure for telescopingly raising and lowering the upper riser 622 relative to the lower riser 620 in a continuously or infinitely adjustable, non-segmented manner. The primary elevator 614 includes one intermediate riser 624 and it is foreseen that additional intermediate rises may be utilized. When the primary elevator 614 includes an intermediate riser 624, the internal motorized structure telescopingly raises and lowers the lower, upper and intermediate risers 620, 622 and 624 relative to one another in a continuously adjustable, non-segmented manner. It is foreseen that the internal motorized structure for telescopingly raising and lowering the risers 620, 622 and 624 may include any suitable continuously adjustable, non-segmented drive known in the art, such as, but not limited to a cable drive, screw drives and hydraulic drives. The head-end lift subassembly 602 includes a powered actuator, electronics and the like, to actuate the primary elevator 614 and the rotation subassembly 616.

The primary elevator 614 is continuously and adjustably movable between a maximum lift or fully extended position, shown on the left side of FIG. 49, and minimum lift or fully lowered position, shown on the left side of FIG. 50. Accordingly, extension of the primary elevator 614 may be adjusted over an infinitely adjustable wide range, for example a distance from about 0.5-inches or less to about 6-inches, 1-foot, 1.5-feet, 2.0-feet, 2.5-feet, 3.0 feet or more. In the fully extended position, the risers 620, 622 and 624 are maximumly outwardly telescoped, or opened, relative to one another, such that a top 625 of the head-end lift subassembly 602 is maximally elevated above the floor F. In contrast, in the fully lowered position, the risers 620, 622 and 624 are maximumly inwardly telescoped, or closed, relative to one another, such that the top 625 of the head-end lift subassembly 602 is as close to the floor F as mechanically possible.

FIG. 48 illustrates an intermediate position of the primary elevator 614, wherein the risers 620, 622 and 624 are intermediately outwardly telescoped relative to one another, such that the top 625 of the head-end lift subassembly 602 is in between the minimum and maximum possible heights. As will be described in greater detail, below, continuously adjustable, non-segmented inward and outward telescoping of the risers 620, 622 and 624, in conjunction with coordinated continuously adjustable, non-segmented cooperative movement of other portions of the patient support and articulation apparatus 600 is associated with positioning the patient, so that the patient's spine will be in a suitable lordotic or kyphotic position for a given surgical procedure or on their side, such as by changing the angle D while substantially maintaining the height H of the point of articulation 601 and optionally or preferably maintaining the patient's torso in a generally horizontal, non-head down position.

The lower riser 620 rests on the footing 618, which includes a housing and at least some of the internal motorized structure of the head-end lift subassembly 602. As shown in FIGS. 57 and 62, the footing 618 extends perpendicularly outward relative to a longitudinal axis B, so as to provide a sturdy support that sufficiently resists sideways tipping of the apparatus 600. The footing 618 includes top and bottom sides 626 and 628, and opposed outer ends 630. A caster 612 extends downwardly from the bottom side 628, adjacent to each of the outer ends 630. The cross-bar 610 is centrally attached to the footing bottom side 628, so as to extend substantially parallel with the longitudinal axis B and the floor F. The cross-bar 610 joins the footing 618 with a footing 618′ of the foot-end lift subassembly 604, described below, so as to hold the footings 618 and 618′ in fixed relation to one another and to provide support to the apparatus 600. It is foreseen that in some embodiments, the apparatus 600 may include nor cross-bar 610, and each of the footings 618 and 618′ may include an inwardly longitudinally extending extension or counter-balance adapted to stabilize the respective footing 618, 618′.

The head-end lift subassembly 602 supports the rotational subassembly 616, which includes an hydraulic piston assembly 632 that rotates or tilts the patient support subassembly 606 and a rotational shaft 634, such as is described elsewhere herein. It is foreseen that other structures such as motors or drives may be used to rotate the subassembly 606. The rotational shaft 634 is substantially parallel with the axis of rotation B, and extends longitudinally inward from the motor housing 632. When the rotational shaft 634 of the head-end and foot-end lift subassemblies 602, 604 are equally spaced above the floor F, the shafts 634 are coaxial with the axis B.

The rotational shaft 634 is rotatably joined with both the patient support subassembly 606 and internal mechanical components of the rotational subassembly 616. The rotational subassembly 616 includes a gear-driven drive or device; however, it is foreseen that alternative drives, such as but not limited to screw-driven, cable-driven and piston-driven drives may be used. Rotating the rotational shaft 634 rotates or tilts the patient support subassembly 606 clockwise or counter-clockwise in a continuous range from about 0° to about 90° in either direction, for example about ±5°, ±10°, ±15°, ±20°, or more relative to axis B. It is foreseen that the drive-device of the rotational subassembly 616 may be located in the top or side of the head-end lift subassembly and in some circumstances, some portions of the drive-device may extend downwardly from the rotational subassembly 616 and into the footing 618. In the illustrated embodiment, a piston 635 is located at the side of the primary elevator 614, that operably rotates the patient support subassembly 606 clockwise or counter-clockwise through a range of plus or minus 20° relative to axis B. Numerous configurations are foreseen. Additionally or alternatively, it is foreseen that a rotational subassembly 616′ may be located at the foot-end lift subassembly 604. The rotational shaft 634 may be passive, and rotate in response to rotation of the patient support subassembly 606 by other apparatus components, such as but not limited to the rotational subassembly 616′. Alternatively, both the rotational subassembly 616 and the rotational subassembly 616′ may actively drive rotation of the patient support subassembly 606, such as by a gear-driven, screw-driven, cable-driven or piston-driven drive known in the art. In some embodiments, the rotational subassembly 616 may be disengaged, or turned off, so as to allow for manual rolling and tilting of the patient support subassembly 606.

The second or foot-end lift subassembly 604 provides for continuous adjustable raising and lowering of the foot-end of the patient support subassembly 606 over an infinitely adjustable range, for example a distance from about 0.5-inches or less to about 6-inches, 1-foot, 1.5-feet, 2.0-feet, 2.5-feet, 3.0 feet or more, in cooperation with other components of the apparatus 600, as described herein. The foot-end lift subassembly 604 also provides for continuous adjustable, non-segmented rotation or tilting of the patient support subassembly 606 over an infinitely adjustable range, for example an amount up to about +5°, ±10°, ±15°, ±20°, or more relative to the axis B, also in cooperation with other components of the apparatus 600, as described herein. The foot-end lift subassembly 604 includes primary and secondary elevators 614′ and 636, a passive rotational subassembly 616′ and a footing 618′. However, it is foreseen that the rotational subassembly 616′ may also be active and include structure similar to the head-end rotational subassembly 616. Similar to the head-end lift subassembly 602, the footing 618′ supports the primary elevator 614′, which supports the rotational subassembly 616′. Unlike the head-end lift subassembly 602, the secondary elevator 636 is operably joined with the rotational subassembly 616′ of the foot-end lift subassembly 604. The primary and secondary elevators 614′ and 636 are individually yet cooperatively operable and continuously adjustable in a non-segmented infinitely adjustable manner.

The primary elevator 614′ is substantially similar to the primary elevator 614 and cooperates with other apparatus components, such as the head-end lift subassembly 602, the secondary elevator 636 and the articulation subassembly 607, such that the angle of articulation D may be modified without a substantial change in height H of the articulation point 601. Accordingly, the primary elevator 614′ includes at least two risers, such as a lower riser 620′ and an upper riser 622′, and an internal motorized structure such as described herein, and provides for modification of a height of the primary elevator 614′ over an infinitely adjustable range, and for example, a distance from about 0.5-inches or less to about 6-inches, 1-foot, 1.5-feet, 2.0-feet, 2.5-feet, 3.0 feet or more. The primary elevator 614′ may include one or more intermediate risers 624′. In the illustrated embodiment, the primary elevator 614′ shown on the right side of FIG. 49 includes one intermediate riser 624′. It is foreseen that in some circumstances, the primary elevator 614′ may include two or more intermediate risers 624′. When the primary elevator 614′ includes an intermediate riser 624′, the internal motorized structure telescopingly raises and lowers the lower, upper and intermediate risers 620′, 622′ and 624′ relative to one another in a continuously and infinitely adjustable, non-segmented manner. It is foreseen that the internal motorized structure for telescopingly raising and lowering the risers 620′, 622′ and 624′ may include any suitable continuously adjustable, non-segmented drive known in the art, such as but not limited to a cable drive, screw drives and hydraulic systems, such as described herein.

Referring again to FIGS. 49 and 50, the primary elevator 614′ is adapted to move between a maximum lift or fully extended position, shown on the right side of FIG. 49, and a minimum lift or fully lowered position, shown on the right side of FIG. 50. In the fully extended position, the risers 620′, 622′ and 624′ are maximumly outwardly telescoped, or opened, relative to one another, such that a top 638 of the foot-end lift subassembly 604 is maximally elevated above the floor F. In contrast, in the fully lowered position, the risers 620′, 622′ and 624′ are maximumly inwardly telescoped, or closed, relative to one another, such that the top 638 of the foot-end lift subassembly 604 is maximally lowered toward the floor F. It is noted that, in the illustrated embodiment, when the primary elevator 614′ is in the least-outwardly telescoped position or configuration thereof, only the lower riser 620′ is visible from the side of the apparatus 600. For example, the intermediate riser 624′ is operable so as to slide downwardly into the lower riser 620′, and the upper riser 622′ is operable so as to slide downwardly into the intermediate riser 624′. In some circumstances, the housing of the rotational subassembly 616′ shrouds at least a portion of the risers 620′, 622′ and 624′. FIG. 48 illustrates an intermediate position of the primary elevator 614, wherein the risers 620′, 622′ and 624′ are intermediately outwardly telescoped relative to one another, such that the top 638 of the foot-end lift subassembly 604 is in between the minimum and maximum possible heights. Numerous riser configurations are foreseen.

As will be described in greater detail, below, inward and outward telescoping of the risers 620′, 622′ and 624′, in conjunction with cooperative movement of other portions of the patient support and articulation apparatus 600 is associated with positioning the patient, so that the patient's spine will be in a suitable lordotic, kyphotic or sideways position for a given surgical procedure. For example, the physician selects the distance H of the patient, or the point of articulation 601, from the floor F that is comfortable for the surgeon to perform the surgery.

The primary elevator 614′ is joined with the footing 618′, which is substantially similar to the footing 618, and which may house a portion of the internal motorized lift structure. The footing 618′ includes a top surface 626′, a bottom surface 628′ and opposed outer ends or surfaces 630′. Casters 612 are attached to the outer ends 630′ of the footing 618′, and the cross-bar 610 is attached to the bottom 628′ of the footing 618′, such as described herein with respect to footing 618. It is foreseen that instead of being connected to the cross-bar 610, the footing 618′ may include a counter-balance such as described elsewhere herein.

The foot-end lift subassembly 604 includes at least a passive rotational subassembly 616′. It is foreseen that the subassembly 604 may include an active or powered rotational subassembly 616′ that is similar to the rotational subassembly 616 of the head-end lift subassembly 602.

Referring to FIGS. 54 and 55, the secondary elevator 636 is joined with the top of the primary elevator 614′ of the foot-end lift subassembly 604, such as, for example, at the housing of the rotational subassembly 616′, such that the secondary elevator 636 in use is operationally raisable or lowerable by the primary elevator 614′. The secondary elevator cooperates with other apparatus components, such as the head-end lift subassembly 602, the primary elevator 614′ and the articulation subassembly 607, such that the angle of articulation of the articulation point 601 may be modified without a substantial change in height H of the articulation point 601.

The secondary elevator 636 extends along the inboard side or face of the foot-end lift subassembly 604, from about the top 638, or top surface, of the foot-end lift subassembly 614, downwards toward the floor F. A top 640 of the secondary elevator 636 may be about coplanar with the top 638 of the foot-end lift subassembly 614, or the top 640 may be somewhat above or below the top 638 of the foot-end lift subassembly 614. The secondary elevator 636 preferably includes a height, or length, sufficient that when the foot-end lift subassembly 604 is in the lowest elevational position, such as is shown in FIG. 56, the bottom 642 of the secondary elevator 636 is located near or adjacent to the top 626′ of the footing 618′.

Referring to FIGS. 54-55, the front or inboard side 644, or face, of the secondary elevator 636 includes an extended vertical slot 646 with a height sufficient to adjustably continuously raise or lower the foot-end of the patient support subassembly 606 in an infinitely adjustable range, for example, a distance of from about 0.5-inches or less up to about 6-inches, up to about 1-foot, up to about 1.5-feet, up to about 2.0-feet, up to about 2.5-feet, up to about 3.0 feet or more. A second rotational shaft 634′ extends toward the patient support subassembly 606 from the vertical slot 646 such that the shaft 634′ is substantially parallel to the axis B or the floor F. The secondary elevator 636 includes a motorized drive, such as is known in the art and described herein, that vertically raises and lowers the shaft 634′ within the slot 646. As shown in FIGS. 50 and 60, when the primary elevator 614′ is in the lowest telescoping position or closed, the secondary elevator 636 lowers the outboard end, generally 652, of the patient support subassembly 606 into close proximity with the floor F, for example, within a few inches of the floor F, such as a distance of about 1-inch or less, about 2-inches, about 3-inches, about 4-inches, about 5-inches, or more. FIG. 54 shows the shaft 634′ in a most elevated position with respect to the secondary elevator 636, wherein the shaft 364′ is at the top 648 of the slot 646. In comparison, FIG. 55 shows shaft 634′ is at the bottom 650 of the slot 646. In use, the secondary elevator 636 is operated independently relative to the primary elevator 614′ or cooperatively with the primary elevator 614′.

The patient support and articulation apparatus 600 includes a patient support subassembly 606 rotatably joined with the head-end and foot-end lift subassemblies 602 and 604. The patient support subassembly 606 includes a head-end support 654 and a foot-end support 654′, each of which has an inboard end and an outboard end. At the outboard ends, the head-end and foot-end supports 654 and 654′ are joined to a respective rotational subassembly 616, 616′ by an intervening translation subassembly 655 and 655′ that includes one or more of an attachment plate 656 and 656′, a cross-bar 658 and 658′, and one or more pivot joints 660 and 660′, such as universal joints or pairs of perpendicularly oriented joints or other suitable pivot structures known in the art. In the illustrated embodiment, such as is shown in FIGS. 54 and 55, the attachment plate 656′ and the cross-bar 658′ are joined by the joint 660′. When the outboard end of the foot-end support 654′ continuously moves between raised and lowered positions, or when the angulation of the pivot point 601 (e.g., angle D) is modified or changed, the attachment plate 656′ and the cross-bar 568′ pivot with respect to each other at the joint 660′. Similar angulation occurs at the head-end between the attachment plate 656 and the cross-bar 658 at joint 660.

Referring to FIGS. 48-60, 62-63, 66, 70, 72, and 75-78, each of the head-end and foot-end supports 654 and 654′ includes a pair of longitudinally extending frames, or spars, 661A and 661B, respectively, for support of the patient. The frames 661A, 661B may be made of any sufficiently strong, rigid material, such as aluminum, carbon fiber, hardened metal, and the like. Preferably, the material of construction of the frames 661A and 661B is non-opaque to x-rays, or radiolucent, so that imaging can occur during surgery. In cross-section, the frames 661A, 661B of the illustrated embodiment are trapezoidal, with the bottom side being wider than the top side (most easily seen in FIGS. 57 and 73), such that the frames 661A, 661B substantially resists torque and tensions applied thereto during movement of the apparatus 600. However, it is foreseen that the frames 661A, 661B may include other cross-sections, such as but not limited to circles, ovals, triangles, rectangles, quadrilaterals and the like.

Each of the frames 661A, 661B includes a longitudinally extending elongate slot or through-bore, generally 662. In the illustrated embodiment, the elongate slot 662 includes a rectangular cross-section and opens downwardly, such as on the bottom side of the cross-section. However, it is foreseen that the elongate slot 662 may have a fourth side, such that the area of the slot 662 is a fully enclosed through-bore, such as is known in the art. Alternatively, the frames 661A, 661B may be tubes with longitudinally extending through-bores 662 therethrough. It is also foreseen that the elongate slot 662 may include other cross-sections, such as but not limited to circles, ovals, triangles, rectangles, quadrilaterals and the like.

Referring to FIG. 57, pairs of frames 661A and 661B are joined at their respective outboard ends, but not at their inboard ends. At the outboard ends, the head-end frames 661A are joined by a perpendicular cross-bar 678 that is joined with the cross-bar 656 of the translation subassembly 655. In contrast, the outboard ends of the foot-end frames 661B are joined by a gearbox 680, which is also part of the angulation subassembly 607. As is discussed in greater detail below, and is shown in FIGS. 50, 55, 56, 60, and 67, the gearbox 680 includes an arch 682, or bowed portion, sized and shaped such that portions 684 of the gearbox 680 may be lowered near to the floor F and around the base support cross-bar 610. It is noted that lowering the outboard end 652 of the foot-end support 654′ sufficiently that the gearbox 680 is located at least partially around the cross-bar 610 enables the head-end support 654 to be maintained in a substantially horizontal orientation, or substantially parallel with the floor F, during angulation of the patient (e.g., a change in angle D), such that the patient's torso may be supported or held in a substantially horizontal or near horizontal orientation, without the head hanging downward and thus reducing side effects of the surgery on the patient.

Referring to FIGS. 66 and 75-78, the frames 661A and 661B are joined at the point of articulation 601, or the axis of rotation C, by a hinge 663. Accordingly, the head-end and foot-end frames, 661A and 661B respectively, provide an open framework, such as is shown in FIGS. 57, 66, 75 and 76, for supporting the patient in a prone, pendulous manner, with the patient's stomach hanging downwardly therethrough. Since the inboard ends of each pair of frames 661A and 661B are not joined together, the patient's abdomen or belly may also depend between the hinges, such as is described below. Additionally or alternatively, rectangular surgical support tops or radiolucent imaging tops, similar to tops 100 and 100′, shown in FIGS. 14, 77 and 78, may be placed on the framework such that the patient can be supported in a supine position or on one of the patient's sides.

As described herein, and shown in FIGS. 57 and 75-78, the patient support subassembly 661A and 661B is substantially a frame, and the head-end support 654 slidably supports the torso trolley 698. As shown in FIGS. 76-78, a number of attachments may be removably attached to the head-end and foot-end supports, or frames 661A, 661B, along a length thereof, and/or to the torso trolley 698 such as but not limited to arm supports, a chest pad, hip pads, regular width and extra wide flat operating boards 100, 100′, regular width and extra wide radiolucent boards 100, 100′, straps and/or slings (not shown) for securing the patient to the frames 661A, 661B, such as are known in the art and described herein. The position or location of such an attachment may be adjusted or modified along a length of the respective frames 661A, 661B, such as to adjust for supporting patients of different sizes.

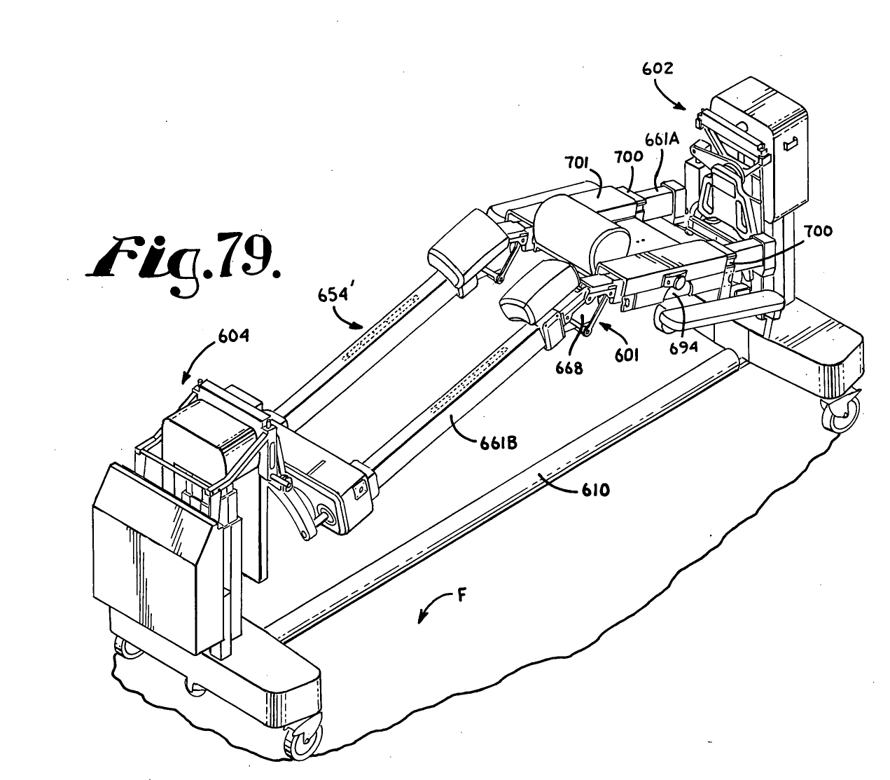

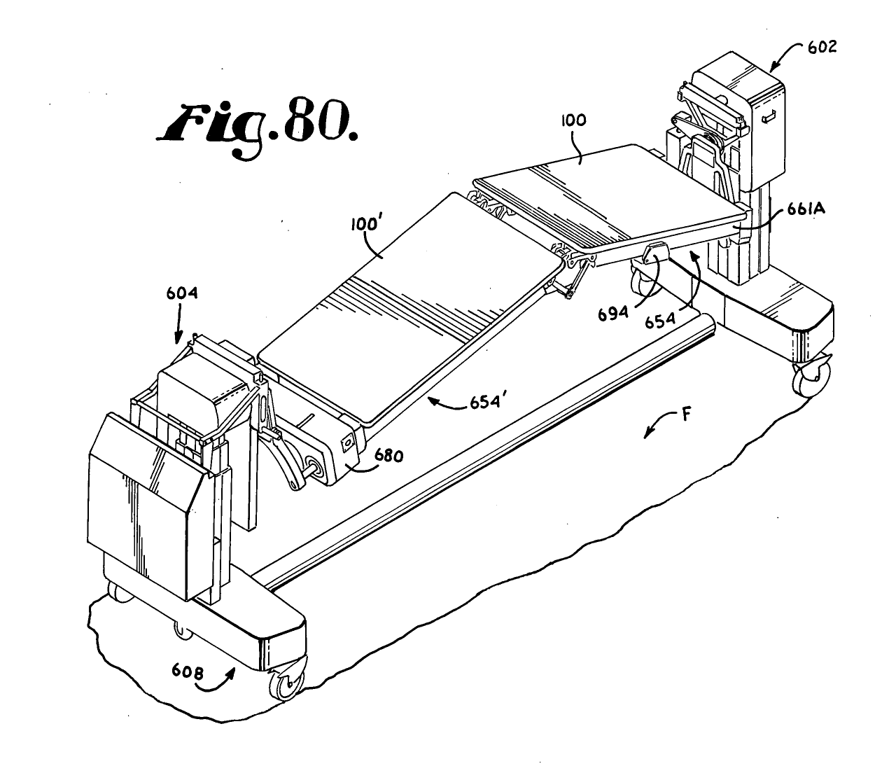

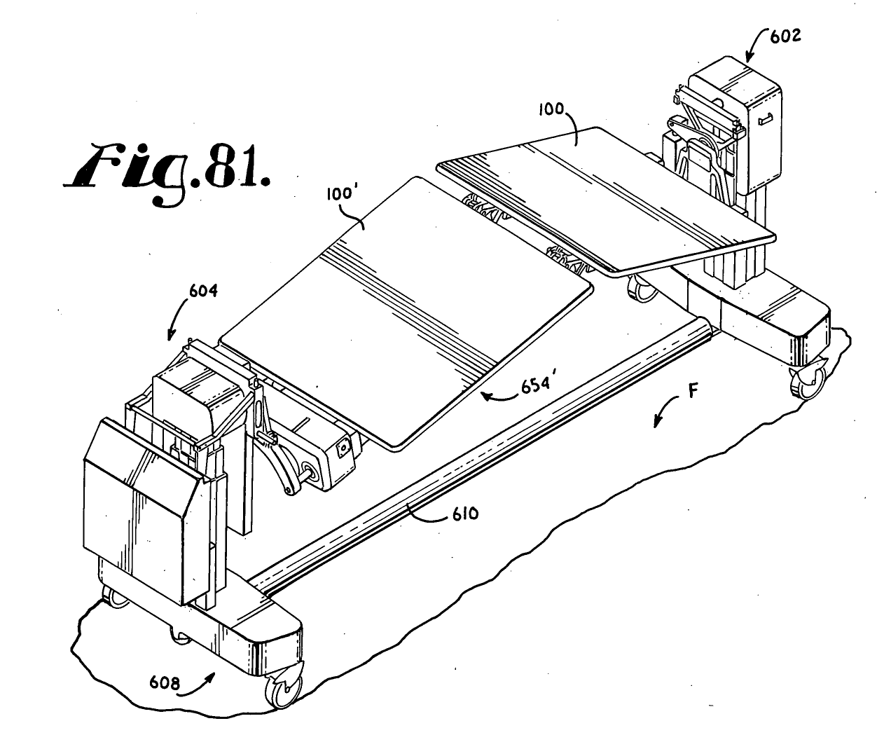

In an exemplary embodiment shown in FIGS. 79-80, regular width flat operating boards 100, 100′ and/or radiopaque boards 100, 100′ are attached to the frames 661A and 6612. In an exemplary embodiment shown in FIG. 81, wide or extra wide flat operating boards 100, 100′ and/or radiopaque boards 100, 100′ are attached to the frames 661A and 661B.

Referring to FIGS. 51-53, 64-65 and 70-75, each hinge 663 includes a pair of knuckles 664 joined by an upper axle 665, upper and lower guide members 667 and 668, a plurality of V-links 669, and a lower axle 665′ pivotably joining the lower guide member 668 and the V-links 669. The hinges 663 can be a wide range of structures that allows articulation between the frames 661A and 661B and are located where it is best for the patient to bend during surgery. For example, the hinges are located so as to selectively flex and extend the patient's hips and/or spine so provide for an amount of lordosis or kyphosis of the patient's lumbar and/or cervical spine selected by the surgeon.

In the illustrated embodiment, each knuckle 664 includes a pair of longitudinally extending, spaced fingers 670. Each of the fingers 670 includes a through-bore 672 that is coaxial with axis C. The upper axle 665 rotatably engages the through-bores 672, such that the upper axle 665 is coaxial with axis C. The respective frames 661A, 661B are joined or engaged by the knuckles 664 at the associated knuckle outboard ends 674. Accordingly, the knuckles 664 can pivot on the upper axle 665 with respect to axis C to thereby modify angle D (see FIGS. 59-60, 70 and 72).

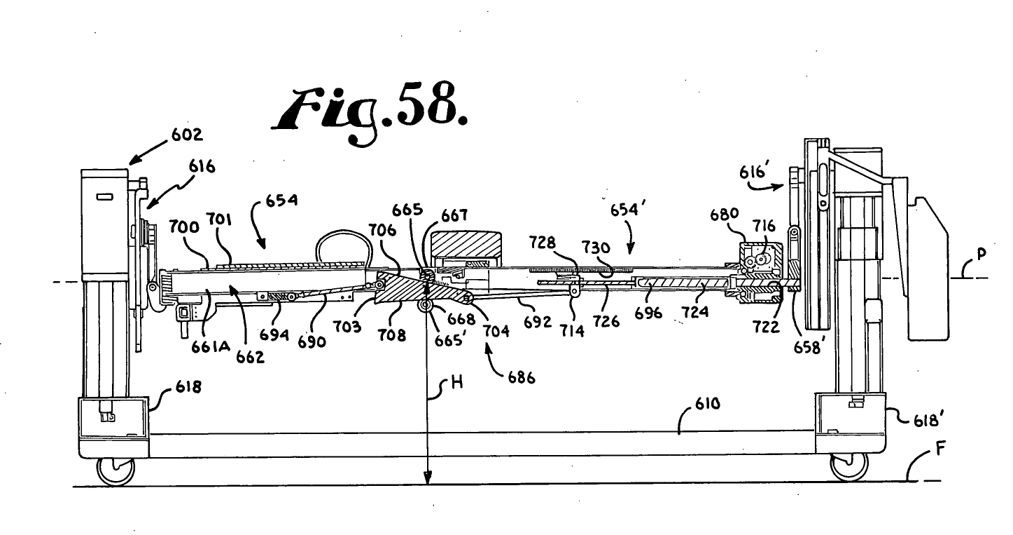

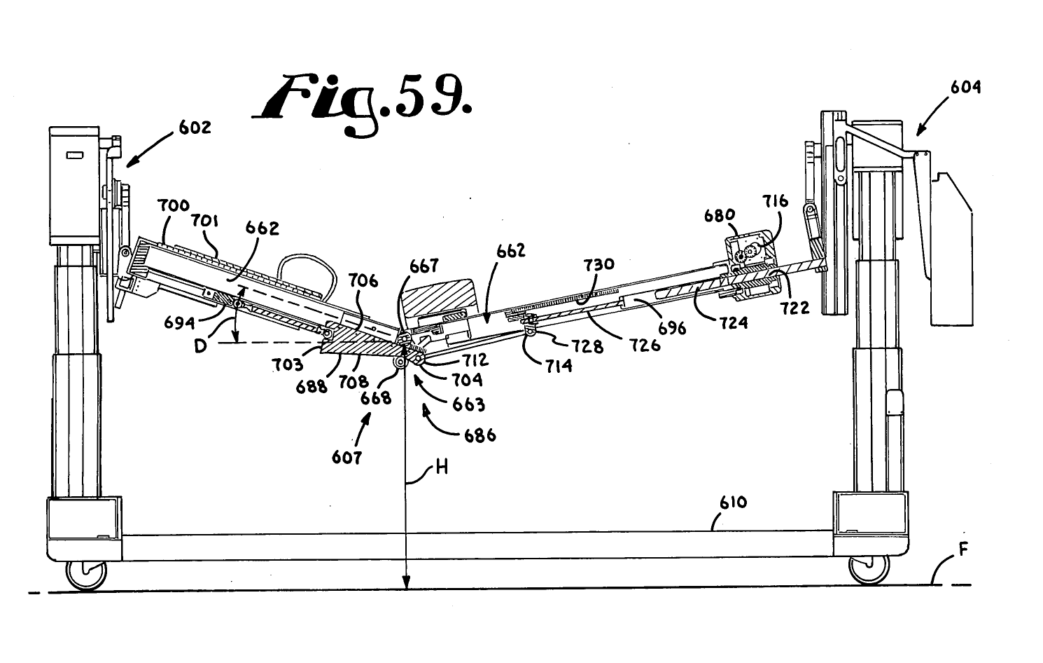

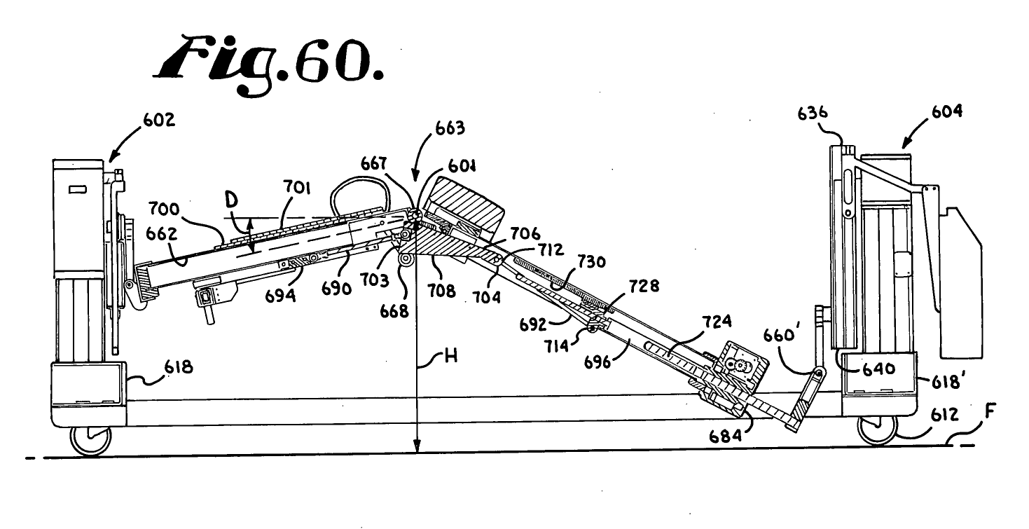

The upper guide member 667 is a structure that includes a through-bore 667A that pivotably receives the upper axle 665 therethrough and engages the top side or portion 706 of the translation wedge 688. In some embodiments, the upper guide member is a roller 667 with a circular cross-section, such as is shown in FIGS. 58-62, 64-66 and 68-69, that is located between the fingers 670 of the joined knuckles 664; and is coaxial with axis C and adapted to pivot freely thereabout independently of the knuckles 664 or of angulation of angle D.

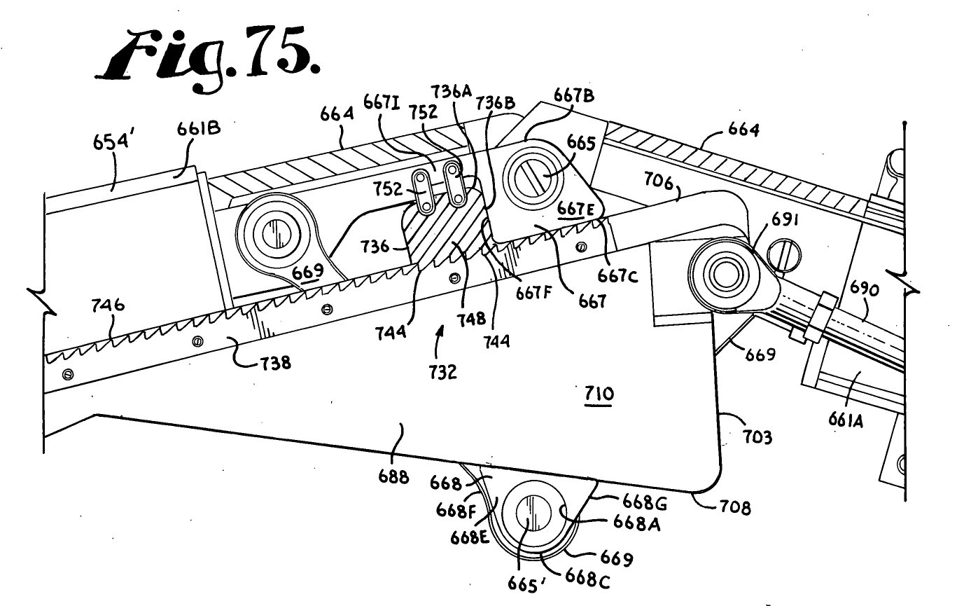

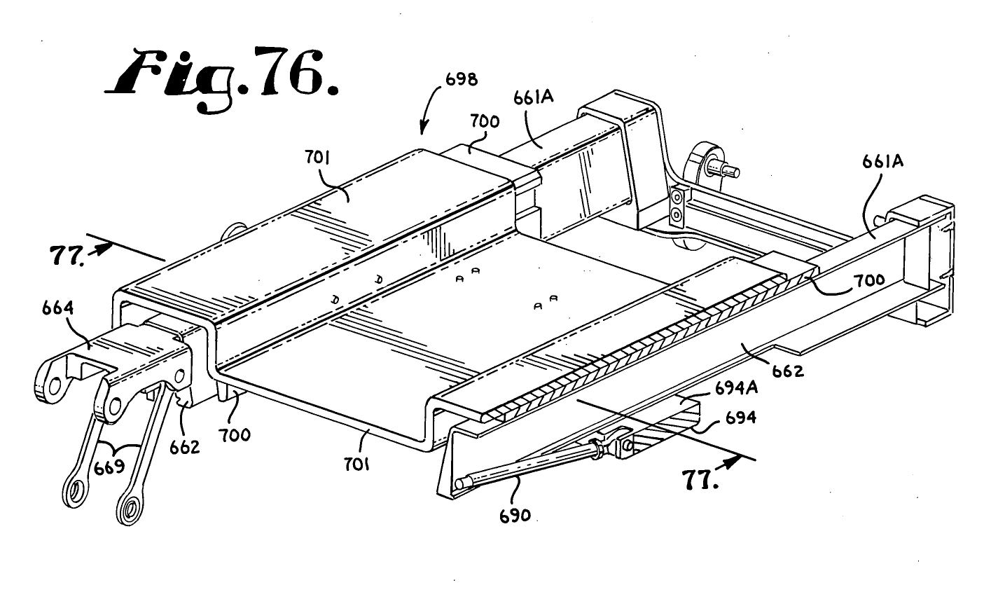

In other embodiments, such as is shown in FIGS. 70-75, the upper guide member 667 is a body or structure 667 with a plurality of sides, such as for example top and bottom sides 667B and 667C, front and rear sides 667D and 667E, and inner and outer sides 667F and 667G. In the illustrated embodiment, a through-bore 667A extends between the inner and outer sides 667F and 667G, is coaxial with axis C, receives the axle 665 therethrough, and adapted to pivot freely thereabout independently of the knuckles 664 or of angulation of angle D. As shown in FIGS. 71 and 72, a guide key structure 667H extends or projects downwardly from the bottom side 667C and extends from about the front side 667D to about the rear side 667E. The guide key 667H is slidingly engaged in an upper locking slot 706A extending along the top portion 706 or side of the translation wedge 688, so as to slide from about the first end 703 of the translation wedge 688 to about the second end 704 thereof. The guide key 667H and the upper locking slot 706A are complementary in shape and size, such that they lock the translation wedge 688 and the upper guide member 667 together, while simultaneously enabling caudad and cephalad sliding movement of the translation wedge 688 with respect to the upper guide member 667, and therefore with respect to the upper axle 665.

It is foreseen that the upper guide member 667 may have an alternatively structure that provides the same function as described herein. The upper guide member 667 may be fabricated of any suitable material that is sufficiently strong so as to withstand the high forces applied thereto during surgery, while still being able to pivot, roll or slide. For example, the upper guide member 667 may be fabricated of hardened metals, carbon fibre, brass, aluminum, and the like, preferably a hardened steel. In some circumstances, the upper guide member 667 may be coated with a hard slick material to facilitate at least one of rolling and sliding, such as is known in the art.

The lower guide member 668 (see FIGS. 58-60, 74 and 75) is a body or structure that includes a through-bore 668A that pivotably receives the lower axle 665′ therethrough and engages the bottom side or portion 708 of the translation wedge 688. In some embodiments, the lower guide member 668 is a roller 668 with a circular cross-section, such as is shown in FIGS. 58-62, 64-66 and 68-69, that is substantially similar to the upper roller 667 in size, shape and fabrication. However, the lower roller 668 may be include alternative sizes, shapes and materials known in the art.

In other embodiments, such as is shown in FIGS. 70-75, the lower guide member 668 is a structure with a plurality of sides, such as but not limited to top and bottom sides 668B and 668C, front and rear sides 668D and 668E, and inner and outer sides 668F and 668G. In the illustrated embodiment, a through-bore 668A extends between the inner and outer sides 668F and 668G and receives the lower axle 665′ therethrough. As shown in FIGS. 71 and 72, a lower guide key structure 668H extends or projects upwardly from the top side 668B of the lower guide member 668 and extends from about the front side 668D to about the rear side 668E thereof. The lower guide key 668H is slidingly engaged in a lower locking slot 708A extending along the bottom portion 708 or side of the translation wedge 688, so as to slide from about the first end 703 of the translation wedge 688 to about the second end 704 thereof. The lower guide key 668H and the lower locking slot 708A are complementary in shape and size, such that they lock the translation wedge 688 and the lower guide member 668 together, while simultaneously enabling caudad and cephalad sliding movement of the translation wedge 688 with respect to the lower guide member 668.

The rod-like V-links 669 pivotably engage the knuckles 664 near their outboard ends 674 and the lower axle 665′, such that an angle E is defined by a pair of intersecting V-links 669 (see FIGS. 50, 52, 53 and 75). Pins pivotably secure the V-links 669 with the knuckles 664 at rear through-bores 676. The V-links 669 are configured and arranged such that the angle E operably moves through a plurality of continuous angles associated with articulation of the patient support subassembly 606. The V-links may be fabricated of any sufficiently resilient material that can withstand high stress and tension. Suitable materials include but are not limited to carbon fiber, hardened metals, aluminum, certain polymers, and the like, and preferably a hardened steel. In some circumstances, the V-links may be fabricated of strong elastic materials, such as certain polymers and composites. Further, in some embodiments, instead of being rod-shaped, the V-links may be braided or non-braided cords, bars, elastic bands and the like, such as is known in the art.

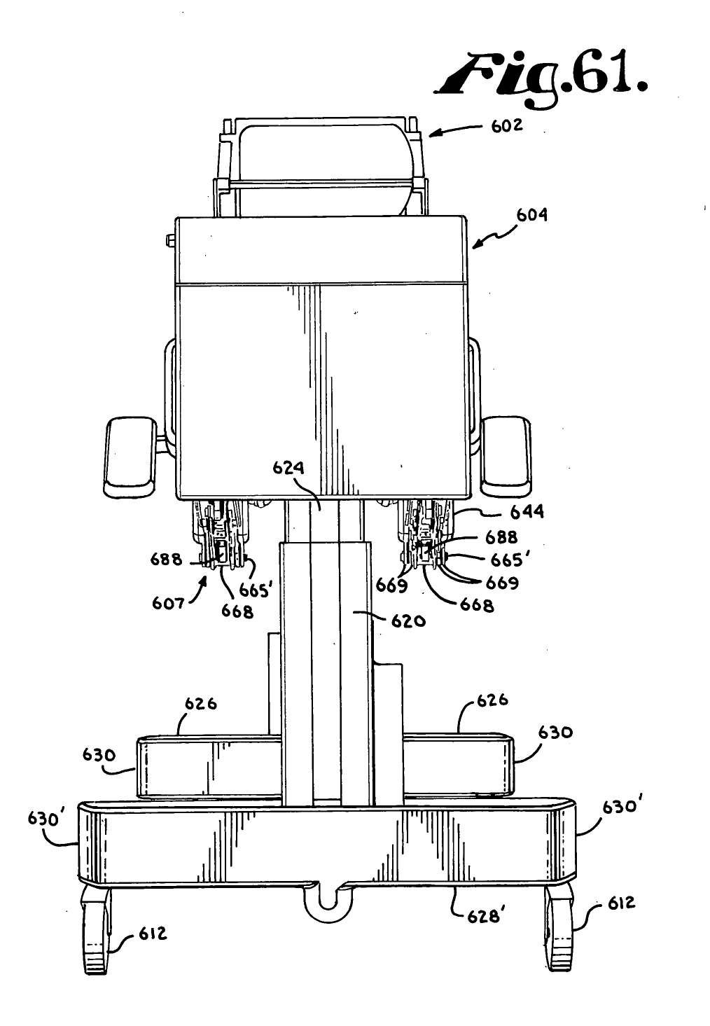

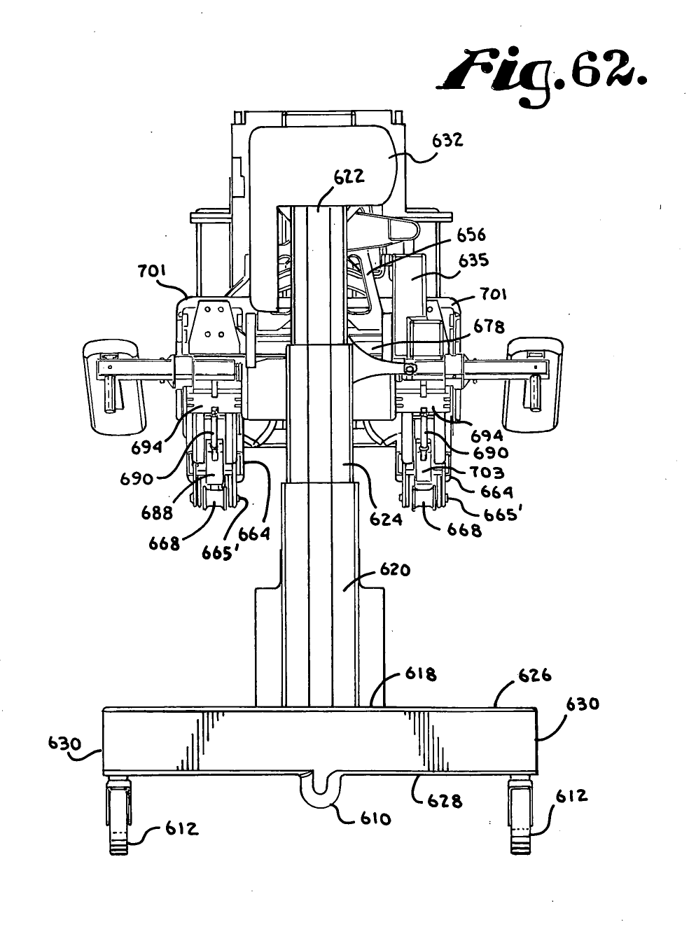

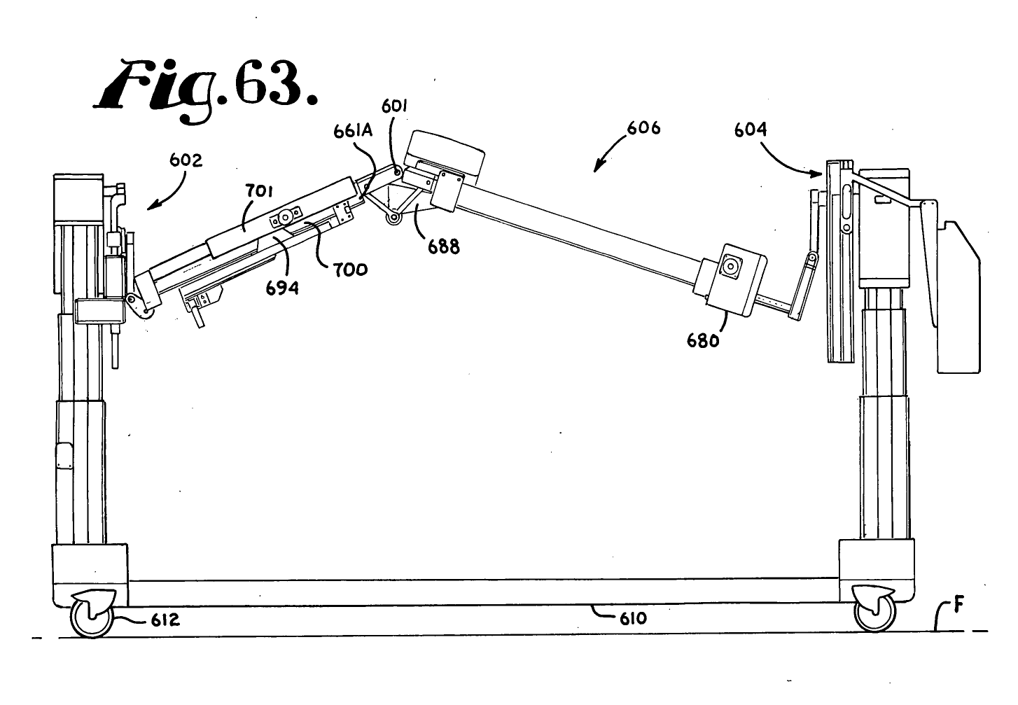

Pairs of V-links 669 engage the lower axle 665′ on at least one side, preferably on both sides, of the lower guide member 668. For example, as shown in FIG. 61, two V-links 669 are joined at each of the left-hand and right-hand ends, or inner and outer ends, of each of the associated lower axles 665′, for a total of four V-links 669 engaging each lower axle 665′. The lower guide member 668 is slidingly received on the lower axle 665′ so as to be located between the engagements or joins of the pairs of V-links 669, such as is shown in FIG. 53. It is foreseen that only two V-links 669 may be used, such as at only left-hand end or the right-hand end of the associated lower axle 665′.

When the guide member 668 is a roller, the lower roller 668 is substantially similar or even identical to the upper roller 667. Accordingly, the lower roller 668 includes a through-bore 668A that pivotably receives the lower axle 665′ therethrough. The lower roller 668 is sized and shaped to pivot freely about the lower axle 665′. In the illustrated embodiment, the lower roller 668 includes a circular cross-section. However, it is foreseen that the lower roller 668 may instead be a slide having a cross-section of another shape, such as but not limited to a rectangle, a polygon, an oval, or the like. It is also foreseen that the lower roller 668 may be an alternative structure that provides the same function as the lower roller 668.

The patient support and articulation apparatus 600 includes an orientation subassembly that includes an individually operable and continuously adjustable articulation subassembly 607 interconnected with the rotation subassemblies 616 and 616′. The orientation subassembly cooperatively rotates and articulates at least a portion of the patient support subassembly 606 so as to allow the patient support subassembly 606 to move through a plurality of infinitely adjustable and non-segmented angular orientations in cooperation with one or more of the primary and secondary elevators 616, 614′ and 636. The articulation subassembly 607 is adapted to articulate the patient support subassembly 606 at the point of articulation 601 up to 90° up or down, for example in an amount of about ±5°, ±10°, ±15°, ±20°, ±25°, +30°, +35°, ±40°, ±45°, ±50° or more with respect to an axis of rotation C and to the subassembly 606 in a horizontal configuration. In some embodiments, the maximum upward breaking position is about 40° to about 45° and the maximum downward breaking position, or an angle of articulation D, is about 30°, relative to axis C, thereby providing a total range of motion of the point of articulation 601 of about 75°. However, it is foreseen that, in some embodiments, the articulation subassembly 607 may move through an infinitely adjustable non-segmented plurality of angular orientations, so as to break upwardly an amount of up to about 90° or more, and as to break downwardly an amount of up to about 90°, or more.

Referring to FIGS. 51-53, 58-60 and 66, the articulation subassembly 607 cooperates with the head-end and foot-end lift subassemblies 602 and 604, so as to continuously and non-segmentedly articulate the patient support subassembly 606 at the point of articulation 601 (e.g., modify angle D) while simultaneously substantially maintaining the height H of the point of articulation 601 relative to the floor F. Additionally, during this articulation at the point of articulation 601, the articulation subassembly 607 cooperates with the head-end and foot-end lift subassemblies 602 and 604 so as to maintain the head-end support 654 of the patient support subassembly 606 in a position that is about parallel with the floor F, such that a patient supported thereon will not be in a substantially head-down position.

The articulation subassembly 607 includes the gearbox 680 operably linked with a pair of tensioned angulation subassemblies, generally 686, that slidingly engage the hinge upper and lower rollers 667 and 668 so as to cause the hinges 663 to break upwardly and downwardly. Each tensioned angulation subassembly 686 includes a tethered translation or angulation wedge 688, the front tether 690, and the tensioned rear tether 692, a sliding bracket 694, and a translation member 696 that engages the gearbox 680. The wedge 688 and the rear tether 692 are constantly under tension so as to urge the wedge 688 at the right in FIG. 59 or toward the end. The front tether 690 may be a rod, a band, a cord, a cable, and the like. The rear tether 692 may be fabricated of any suitable elastic or non-elastic material known in the art.

As shown in FIGS. 66, 70, 78 and 79, the trolley sliding brackets 694, or trolley sliders, slidably engage the associated frame 661A from the bottom thereof, such that the sliding brackets 694 at least partially surround the associated frames 661A, including portions of the bottom and two sides of the frames 661A. For example, in the illustrated embodiment, the frame 661A includes a trapezoidal cross-section, wherein the cross-section is taken perpendicular to a longitudinal axis thereof. Each of the sliding brackets 694 includes an inner surface 694A that defines a trapezoidal cross-section sized and shaped to slidingly mate with the frame 661A, wherein the cross-section is taken perpendicular to a longitudinal axis thereof. It is noted that since the frame 661A and the sliding brackets 694 each include complementary trapezoidal cross-sections, with the bottom sides being substantially wider than the top sides, the sliding brackets 694 may not be pulled or pushed downward and off of the associated frame 661A.

The sliding brackets 694 are adapted to slide in the cephalad and caudad directions along the frames 661A, or along a length of the frames 661A. In some circumstances, the inner surfaces 694A of the sliding brackets 694 engaging the frame 661A are lubricated, such as to facilitate such sliding movement. Each sliding bracket 694 is engaged by a front tether 690 that pushes or pulls the sliding bracket 694 in the cephalad and caudad directions in response to actuation of the tensioned angulation subassembly 696, such as is described below.

Referring to FIGS. 66, 71, 78 and 79, the sliding brackets 694 are releasably engaged by a torso trolley, generally 698, that rests on the frames 661A and includes a pair of slide channel members 700 and a chest slide 701. In the illustrated embodiment, each of the slide channel members 700 is adapted to releasably slidingly engage the tops and sides of an associated frame 661A, and includes sliding bracket receiving portion 702 sized and shaped to releasably mate with the sliding bracket 694 associated with the frame 661A. It is foreseen that the slide channel members 700 may have other shapes and sizes, depending upon the size and shape of at least one of the frame 661A and the sliding brackets 694, so long as they fulfill the function described herein. Cephalad and caudad movement of the sliding brackets 694 along the associated frames 661A translates the slide channel members 700 along the frames 661A, which in turn moves the chest slide 701 in the cephalad and caudad directions, such as is described in greater detail below. It is noted that each slide channel member 700 may be easily removed from the associated frame 661A simply by lifting the channel member 700 off of the frame 661A.

The translation wedge 688, also referred to as an angulation wedge 688, includes first and second ends 703 and 704, top and bottom portions 706 and 708, and a pair of opposed faces 710. In the illustrated embodiment, the translation wedge 688 is generally thin, flat and triangular in shape. However, the translation wedge 688 may have any other shape so long as it fulfills its function as described herein. For example, it is foreseen that the translation wedge 688 may be a cam, a roller, a polygon, a sphere, and the like. The translation wedge 688 may be fabricated of any sufficiently strong and resilient material able to withstand high stress and tension resulting from the apparatus 600 supporting a patient weighing up to at least 500-pounds. Suitable materials include but are not limited to aluminum, hardened metals and carbon fiber. It is foreseen that the top and bottom portions 706 and 708 may be treated to increase or decrease lubrication, as is known in the art.

Referring to FIGS. 58-60, 66, 68-69, and 74-76, the first end 703 of the translation wedge 688 engages the front tether 690 and the second end 704 engages the rear tether 692, or linkage strut. The translation wedge top and bottom portions or sides 706 and 708 slidably engage the upper and lower guide members 667 and 668, respectively. For example, when the upper and lower guide members 667 and 668 are rollers, the upper guide member 667 rolls along the wedge top portion 706, such that the roller surface 742 frictionally engages the top portion 706, and the lower guide member 668 rolls along the wedge bottom portion 708, such that the roller surface 668B frictionally engages the bottom portion 708. In another example, when the upper and lower guide members 667 and 668 are bodies with key guides 667H and 668H, the upper key guides 667H slidingly engages the wedge upper locking slot 706A, such that the upper guide member 667 bottom side 667C slidingly engages the wedge top portion 706; and the lower key guides 668H slidingly engages the wedge lower locking slot 708A, such that the lower guide member 668 top side 668B slidingly engages the wedge bottom portion 708.

The translation wedge 688 is pulled and pushed between the upper and lower guide members 667 and 668 by the rear tether 692, which in turn is pushed and pulled by the translation member 696 in response to actuation of the gearbox 680, as is described herein. The wedge 688, because of the weight of the structure acting thereon, is always urged away from the rear tether 692, so as to place tension thereon.

The rear tether 692 includes first and second ends 712 and 714, and may be a rod, a band, a cord, a cable, and the like. The rear tether 692 may be fabricated of any suitable flexible but generally non-stretchable or non-elastic material known in the art. The rear tether 692 is tensioned between the second end 704 of the translation wedge 688 and the translation member 696. As shown in FIG. 58, the first end 712 of the rear tether 692 engages the second end 704 of the translation wedge 688, and the second end 714 of the rear tether 692 engages the translation member 696 by an intervening translation nut member 728. The rear tether 692 is pulled or released in the cephalad and caudad directions, respectively, through the translation member 696.

The translation member 696 engages the translation nut member 728 and the gearbox 680. As shown in FIGS. 66 and 67, the gearbox 680 includes a motorized gear assembly, generally 716, and a motor 718. In the illustrated embodiment, the gear assembly 716 includes a worm gear. However, it is foreseen that any type of gear assembly 716 may be used, so long as it can move or translate the translation member 696 in the cephalad and caudad directions. The translation member 696 also includes an outer translation structure 720, such as a tube or a cylinder with a central through-bore, that passes through a through-bore 722 in the gear assembly 716. An inner translation structure 724, such as a translation rod or bar, slides in and out of the outer translation structure 720. A translation screw 726 is secured to an end of the inner translation structure 724 within the outer translation structure 720. The translation screw 726 engages the translation nut member 728 that engages the second end 714 of the rear tether 692. The translation nut member 728 moves or translates along a translation track 730 located within the elongate slot 662 of the frame 661B, in the cephalad and caudad directions, in response to actuation of the translation screw 726.

To articulate the patient support subassembly 606 in an upwardly or downwardly breaking configuration, or to align the subassembly 606 in the first plane P, the gearbox 680 is actuated. Actuation of the gearbox 680 moves the translation wedge 688 between the upper and lower rollers 667 and 668, in either a caudad direction by drawing the tether 692 toward the gearbox 680 or in a cephalad direction by allowing the tether 692 slack so that the tension at the wedge 688 pulls the rear tether away from the gearbox 680. Upward and downward breaking is associated with a distance between the guide members 667 and 668, the distance being generally perpendicular to the floor F. When the guide members 667 and 668 are closer together, the hinge 663 breaks downwardly. When the guide members 667 and 668 are farther apart, the hinge 663 breaks upwardly. Gravity and the weight of the patient facilitate downward breaking. When the translation wedge 688 moves in a cephalad direction, the guide members 667 and 668 move or slide along the top and bottom portions 706 and 708 towards the translation wedge second end 704, such that the guide members 667 and 668 are moved closer together with respect to the translation wedge 688, thereby causing the patient support subassembly 606 to break downwardly. When the translation wedge 688 moves in a caudad direction, the guide members 667 and 668 move or slide along the top and bottom portions 706 and 708 towards the translation wedge first end 703, the guide members 667 and 668 are pushed apart, thereby causing the patient support subassembly 606 to break upwardly. Accordingly, a distance between the upper and lower guide members 667 and 668 increases or decreases as the translation wedge 688 moves in the caudad and cephalad directions, respectively.

It is noted that the degree of angulation D is associated with the shape of the translation wedge 688 and the spacial relationship between the translation wedge 688 and the guide members 667 and 668, such as but not limited to the length of the top and bottom portions 706 and 708 and the size of an angle defined by the top and bottom portions 706 and 708 and the second end 704. For example, longer top and bottom portions 706 and 708 and/or a greater angle facilitate moving the guide members 667 and 668 farther apart, which in turn facilitates a greater amount of angulation of the patient support subassembly 606. In a certain embodiment, movement of one inch of the wedge 688 relative to the guide members 667 and 668 translates to ten degrees of angulation; however, it is foreseen that this could be varied greatly, for example one inch could translate to 2, 5, 20 or any selected degrees.

FIG. 58 shows the patient support subassembly 606, or the head-end and foot-end supports 654 and 654′, aligned in the first plane P. When the patient support subassembly 606 is aligned with the first plane P, the upper and lower guide members 667 and 668 are located medially between the first and second ends 703 and 704 of the translation wedge 688. Concurrently, the trolley sliding bracket 694 is located medially along the length of the head-end support 654. Each of the sliding brackets 694 engages an associated sliding channel member 700, which are both engaged by the chest slide 701. The inner translation structure 724 is moved into the outer translation structure 720, the translation nut member 728 is medially along the translation track 730, and the gearbox 680 is located near the cross-bar 658′.

FIG. 59 shows the patient support subassembly 606 in a downwardly breaking configuration, wherein the hinge 663 is located at or near the first plane P and the frame 661A, 661B outboard ends have been raised a distance above the plane P, such as by outward, or upward, telescoping of the head-end and foot-end lift subassemblies 602 and 604. The apparatus 600 is adapted to move in a smooth, continuously and infinitely adjustable, non-segmented manner between the configuration of FIG. 58 and the configuration shown in FIG. 59 and back again. In the configuration shown in FIG. 59, the upper and lower guide members 667 and 668 are located near the second end 704 of the translation wedge 688. When moving from the configuration of FIG. 58 to the configuration of FIG. 59, the sliding brackets 694 move “up hill” in a cephalad direction, or towards the head-end lift subassembly 602. Movement of the sliding brackets 694 moves the torso trolley 698, or the sliding channel members 700 and the chest slide 701, towards the head-end lift subassembly 602 a distance associated with the amount of downward breaking or angulation of angle D, such as at the point of articulation 601. The translation wedge 688 is sized and shaped such that when the hinge 663 breaks downwards, the torso trolley 698 is moved, is pushed or slides towards the head-end lift subassembly 602, or “up hill.” It is noted that in the configuration of FIG. 59, the translation nut member 728 has moved along the translation track 730, towards the hinges 663. Accordingly, the translation wedge 688 has been drawn between the guide members 667 and 668, which roll along the top and bottom portions 706, 708 until the translation wedge second end 704 is located near the guide members 667 and 668. The translation nut member 728 has also moved along the translation screw 726 towards the head-end lift subassembly 602, which is actuated by rotation of the translation screw 726. Further, actuation of the gearbox 680 rotates the translation screw 726 and moves the inner translation structure 724 away from the foot-end lift subassembly 604, effectively lengthening the foot-end lift subassembly 604.

In an exemplary embodiment, the apparatus 600 includes a patient support subassembly 606, with a centrally located pair of spaced apart hinges 663, a chest slide or torso trolley 698 adapted to translate along a length of the patient support subassembly 606 and a linkage, such as but not limited to a linkage structure 686, that links or joins the hinges 663 with the chest slide 698. Further, such breaking of the hinges 663 is linked with translation of the chest slide 698 along a length of the patient support subassembly 606, such as is described in greater detail elsewhere herein. In a further embodiment, when the hinges 663 break upwardly, the linkage causes the chest slide 698 to translate toward the hinges 663. Similarly, when the hinges 663 break downwardly, the linkage causes the chest slide 698 to translate away from the hinges 663. In addition to the linkage structure 686 described herein, alternative mechanisms for linking the movement of the hinges 663 with the movement of the chest slide 698 are foreseen. For example, such a linkage may be a physical structure, such as is described herein, or a computer software synchronization.

FIG. 60 shows the patient support subassembly 606 in an upwardly breaking configuration, wherein the hinge 663 is located at or near the first plane P and the frame 661A, 6961B outboard ends have been lowered a distance below the plane P, such as by inward, or downward, telescoping of the head-end and foot-end lift subassemblies 602 and 604. The apparatus 600 is adapted to move in a smooth, continuously adjustable, non-segmented manner between the configuration of FIG. 58 and the configuration shown in FIG. 60 and back again. In the configuration shown in FIG. 60, the upper and lower guide members 667 and 668 are located near the first end 703 of the translation wedge 688. It is noted that the trolley sliding brackets 694 is again moved “up hill”, in a caudad direction, or towards the foot-end lift subassembly 604. Movement of the sliding brackets 694 moves the torso trolley 698, or of the associated sliding channel members 700 and the chest slide 701, away from the head-end lift subassembly 602 a distance associated with the amount of downward breaking or angulation of angle D. The translation wedge 688 is sized and shaped such that when the hinge 663 breaks upward, the torso trolley 698 slides towards the foot-end lift subassembly 604, also up hill. It is noted that in the configuration of FIG. 60, the translation nut member 728 has moved along the translation track 730, towards the foot-end lift subassembly 604. Accordingly, the translation wedge 688 has been pulled between the guide members 667 and 668, until the first end 703 of the translation wedge 688 is located near the guide members 667 and 668. The translation nut member 728 has also moved along the translation screw 726 towards the foot-end lift subassembly 604, which is actuated by rotation of the translation screw 726. Further, actuation of the gearbox 680 rotates the translation screw 726 and moves the inner translation structure 724 towards the foot-end lift subassembly 604, effectively lengthening the foot-end lift subassembly 604. It is again noted that when the apparatus 600 is in the configuration shown in FIG. 60, wherein the hinge 663 is in an upwardly breaking configuration and the foot-end lift subassembly 604 is in its lowest possible configuration and the primary and secondary elevators are both maximally lowered, the intersection of the inner translation member 728 and the cross-bar 658′ are substantially near the floor F, such that the ends of the cross-bar 658′ pass around the cross-bar 610 of the base support 608 and portions 684 of the gearbox 680 pass around the cross-bar 610 so as to be located near the floor F, instead of being located above the cross-bar 610. This enables maintaining the head-end support 654 in a substantially horizontal position, relative to the floor F, such as by raising the head-end lift subassembly 602, while providing the amount or degree of angulation at the point of angulation 601 required by a given surgical procedure.

Since the distance between each sliding bracket 694 and the associated translation wedge 688 is fixed by the length of the front tether 690, the distance that the sliding brackets 694 move, or that the torso trolley 698 moves, is associated with the change in angulation of angle D at the point of articulation 601. The change in angle D is associated with the location of the translation wedge 688 relative to the upper and lower guide members 667 and 668. Accordingly, the greater the change in angle D, the farther the torso trolley 698 is moved.

The apparatus 600 includes a failsafe structure, generally 732, adapted to operably engage the articulation subassembly 607 in the event of catastrophic failure of the apparatus 600. Catastrophic failure includes but is not limited to physical or mechanical breaking, or wearing out, of a hinge 663, a V-link 669, the translation wedge 688, a front or rear tether 690, 692, loosening of a screw or bolt, wearing out of a gear or motor, and electrical failure. In an exemplary embodiment, actuation of the failsafe 732 locks the position of the patient support subassembly 606 at axis C, such that additional or further articulation about axis C is substantially blocked. It is foreseen that numerous additional failsafe devices known in the art can be incorporated into the apparatus 600, into various components such as the head-end and foot-end lift subassemblies 602 and 604, and the patient support subassembly 606, such as to prevent improper disconnection of the patient support subassembly 606 from the base, and the like.

Referring to FIGS. 65, 68 and 69, in an illustrated embodiment of the invention, wherein the guide members 667 and 668 are rollers, the failsafe structure 732 is associated with the hinges 663 and the translation wedge 688. The failsafe structure 732 includes at least one, preferably two guides 734, a ratchet locking structure 736, pawl or ratchet break 735, and a toothed ratchet strip 738, or rack, attached to at least one face 710 of the translation wedge 688 adjacent to the top portion 706 thereof. The ratchet locking structure 736 is located between two guides 734 and includes a gripping surface 740 sized and shaped to grippingly engage the surface 742 of the upper guide members or roller 667. The ratchet locking structure 736 also includes a plurality of ratchet teeth 744 sized and shaped to engage the ratchet teeth 746 of the ratchet strip 738. The failsafe structure 732 may include a device for preventing engagement of the teeth 744 and 746, such as but not limited to a solenoid 748. For example, a solenoid 748 such as shown in FIG. 65 may bias the ratchet locking structure 736 upwardly, so as to block engagement of the teeth 744 and 746. The ratchet locking structure 736 is biased downwardly, such as by a leaf spring 750, so as to facilitate engagement of the teeth 744 and 746, and it is foreseen that this function could be provided by a solenoid or other device.

Referring to FIGS. 70-72, in another illustrated embodiment of the invention, wherein the guide members 667 and 668 are sliding bodies 667 and 668 with key guides structures 667H and 668H that slidingly matingly engage an associated locking slot 706A and 708A of the translation wedge 688, the failsafe structure 732 is still associated with the hinges 663 and the translation wedge 688. In this embodiment, the failsafe structure 732 includes a ratchet locking structure 736 with at least one tooth 744 and a toothed ratchet strip 738, or rack, attached to at least one face 710 of the translation wedge 688 adjacent to the top portion 706 thereof. The at least one ratchet tooth 744 is sized and shaped to engage the ratchet teeth 746 of the ratchet strip 738. Preferably, a toothed ratchet strip 738 is located on each face 710 of the translation wedge 688 top portion 706, and the ratchet locking structure 736 includes at least one tooth 744 sized and located for engaging each of the strips 738. Accordingly, the ratchet locking structure 736 slides or rides along the top portion 706 of the translation wedge 668, as the wedge 668 is moved back and forth between the guide members 667 and 668. The failsafe structure 732 includes a solenoid 748, or similar device, that prevents engagement of the teeth 744 and 746, when a catastrophic failure has not occurred, such that the hinge 663 can move from a maximally upwardly broken configuration toward a planar or a downwardly broken configuration. For example, a solenoid 748 such as shown in FIGS. 68, 69 and 75 may bias the ratchet locking structure 736 upwardly, so as to block engagement of the teeth 744 and 746. The ratchet locking structure 736 is movably attached to the upper guide member 667 by linkages 752, which downwardly bias the ratchet locking structure 736. For example, the linkages 752 movably join the top side 736A of the ratchet locking structure 736 with one or more forwardly extending fingers 6671 of the upper guide structure 667, such that the rear side 736B of the ratchet locking structure 736 is located adjacent to the front side 667F of the upper guide member 667. In some embodiments, the linkages 752 may join the rear side 736B with the front side 667F. Numerous configurations are foreseen.

During normal operation of the apparatus 600, when the translation wedge 688 is moved towards the foot-end lift subassembly 604, the ratchet locking structure 736 slides along the ratchet strip 738, such that the teeth 744 and 746 do not become engaged. Alternatively, the ratchet locking structure 736 may be biased upwardly, such as by the solenoid 748, so that the teeth 744 and 746 do not become engaged. When the translation wedge 688 is moved towards the head-end lift subassembly 602, the ratchet locking structure 736 is biased upwardly, such as by the solenoid 748, so that the teeth 744 and 746 do not become engaged.

In the event of a catastrophic failure of the apparatus 600, for example power failure, the solenoid 748 no longer maintains separation and the teeth 744 of the downwardly biased ratchet locking structure 736 engage the ratchet strip teeth 746. Since the translation wedge 688 is biased towards the head-end lift subassembly 602 by downward forces from the weight of the patient on the assembly 600, and the solenoid no longer biases the ratchet locking structure away from the translation wedge 688, the fail-safe locks the position of the translation wedge 688 with respect to the guide members 668 and 668. For example, when the guide members 667 and 668 are rollers, the translation wedge 688 pulls or pushes the ratchet locking structure 736 between the upper roller 667 and the translation wedge top portion 706. The gripping surface 740 non-slidingly engages the surface 742 of the upper roller 667 and the ratchet teeth 744 of the ratchet locking structure 736 lockingly engages the ratchet strip 738, thereby locking, fixing or binding-up translation wedge 688 and the upper roller 667, and substantially blocking further movement or articulation of the articulation subassembly 607. In another example, when the guide members 667 and 668 are the bodies of FIGS. 70-72 with key guide structures 667H and 668H, the ratchet locking structure 736 is biased downwardly, such as by the linkages 752, such that the teeth 744 of the ratchet locking structure 736 engage the teeth 746 of the ratcheted strips 738. As the hinge 663 moved in a downwardly breaking direction, the upper guide member 667 hits or bumps up against the locked ratchet locking structure 736, such that the translation wedge 688 is blocked from moving further in a foot-ward direction, and further downward breaking of the hinge 663 is substantially blocked thereby.

The apparatus 600 includes a powered actuator and electronics such as are known in the art and described herein.

It is to be understood that while certain forms of the present invention have been illustrated and described herein, it is not to be limited to the specific forms or arrangement of parts described and shown.

1. An apparatus for supporting and positioning a patient during a medical procedure, comprising:
a) a patient support subassembly having a centrally located pair of spaced apart hinges adapted to a break upwardly and downwardly;
b) a chest slide adapted to translate along a length of the patient support subassembly; and
c) a linkage linking the hinges with the chest slide, such that breaking of the hinges is linked with translation of the chest slide.

2. The apparatus according to claim 1, wherein:
a) when the hinges break upwardly, the linkage causes the chest slide to translate toward the hinges; and
b) when the hinges break downwardly, the linkage causes the chest slide to translate away from the hinges.

3. An apparatus for supporting and positioning a patient during a medical procedure, the apparatus comprising:
a) a hinge subassembly joining a head portion of an open frame with a foot portion of the frame, the hinge subassembly being located near the middle of a patient supported on the frame and being adapted to controllably angulate in both upward and downward directions and also to return to a non-angled position; and
b) a hinge driver subassembly cooperating with the hinge subassembly, the hinge driver subassembly being translated in a cephalad direction when the hinge subassembly angles downwardly and also being translated in a caudad direction when the hinge subassembly angles upwardly, so as to position the patient in a plurality of prone and non-prone positions.

4. The assembly according to claim 3, wherein
a) the hinge driver subassembly is selectively lockable.

5. The assembly according to claim 4, wherein
a) the hinge driver subassembly includes a fail-safe locking structure adapted for locking the frame in the event of a catastrophic failure of the apparatus.

6. The assembly according to claim 3, further comprising
a) a torso trolley subassembly associated with the frame head portion and adapted for supporting and translating the torso of the patient along a length of the frame head portion in the cephalad and caudad directions and cooperating with the hinge subassembly and hinge driver subassembly to translate the torso in a cephalad direction when the hinge angles downwardly and also to translated the torso in a caudad direction when the hinge angles upwardly.

7. The assembly according to claim 3, further comprising
a) a translation compensation subassembly located inside of the frame foot portion and cooperating with the hinge subassembly and the hinge driver subassembly to translate the foot portion in a cephalad direction when the hinge subassembly angles either upwardly or downwardly and also to translate the foot portion in a caudad direction when the hinge subassembly moves toward the non-angled position.

8. The assembly according to claim 3, further comprising
a) a pair of space apart head and foot primary motorized vertical end elevators joined with outward ends of the frame head and foot portions; and
b) an infinitely adjustable secondary motorized vertical end elevator adapted for better alignment and orientation of the frame so as to allow additional lowering of the outer end of the frame foot portion beyond an amount of lowering provided by the primary elevators.

9. The apparatus of claim 3, wherein
a) the hinge subassembly includes a maximum upward angulation of between about 40° and about 45°.

10. The apparatus of claim 3, wherein
a) the hinge subassembly includes a maximum downward angulation of about 30°.

11. The apparatus of claim 3, wherein
a) the hinge subassembly includes a total range of motion of about 75°.

12. An apparatus for supporting a patient during a medical procedure, the apparatus comprising:
a) an open frame having a single breaking location around the middle of a patient positioned on the frame, the frame being outwardly connected to and supported between spaced apart head and foot primary motorized vertical end elevators; and
b) an additional motorized vertical end elevator adapted for infinite adjustability to better align and orient the frame so as to allow for additional vertical adjustability of a frame outer end beyond an amount provided by the primary elevators; wherein
c) the frame can controllably break and bend, upwardly and downwardly, and is adapted to manipulate the patient in a plurality of selectively lockable prone and non-prone positions in cooperation with a frame translation compensation mechanism located in-between the primary end elevators and the frame, while also cooperating with the primary and secondary motorized end elevators to move the patient vertically while the patient is positioned on the frame.

13. The apparatus according to claim 12, wherein
a) the translation compensation mechanism cooperatively moves an outer end of the frame away from the respectively connected end elevator in response to upward or downward bending of the frame at the frame breaking location; and
b) the translation compensation mechanism cooperatively moves the outer end of the frame toward the respectively connected end elevator in response to unbending of the frame at the frame breaking location, so as to move the patient between at least two of the prone and non-prone positions.

14. The apparatus according to claim 12, wherein:
a) the translation compensation mechanism is located within the frame.

15. The apparatus according to claim 12, further comprising:
a) an angulation subassembly including a pivot associated with the frame breaking location and a driver; wherein
b) downward bending of the frame breaking location translates the driver away from the frame and upward bending of the frame translates the driver toward the frame breaking location, so as to manipulate a patient supported on the frame in a plurality of prone and non-prone positions.

16. The apparatus according to claim 15, further comprising:
a) a chest slide associated with a head end of the frame and tethered to the pivot; wherein
b) upward bending of the frame translates the chest slide toward the pivot and downward bending of the frame translates the chest slide away from the pivot, so as to manipulate the patient in the plurality of prone and non-prone positions.

17. The apparatus according to claim 15, further comprising:
a) a fail-safe mechanism adapted to substantially lock the frame in the event of a catastrophic failure of the apparatus.

18. The apparatus of claim 15, wherein
a) the pivot includes a maximum upward bending position of between about 40° and about 45°.

19. The apparatus of claim 15, wherein
a) the pivot includes a maximum downward bending position of about 30°.

20. The apparatus of claim 15, wherein
a) the pivot includes a total range of motion of about 75°.

21. An apparatus for moving a patient between at least two of a plurality of selectively lockable prone and non-prone positions, the patient being supported on a frame connected outwardly to and supported between a pair of opposed primary vertical end elevators adapted to raise and lower a connected end of the frame, the frame including head and foot portions, the apparatus comprising:
a) a continuously adjustable angulation subassembly with a central pivot and located around a middle of the patient, the angulation subassembly being adapted to controllably break and angle the frame both upwardly and downwardly and to de-angle the frame to a position wherein the frame head and foot portions are located within a single plane, wherein at least a portion of the angulation subassembly is translated in a cephalad direction when the angulation subassembly angles the frame downwardly and also is translated in a caudad direction when the angulation subassembly angles the frame upwardly;
b) a chest slide subassembly supported by a head end of the frame and cooperating with the angulation subassembly to translate a torso of the patient in response to manipulation of the patient between at least two of a plurality of selectively lockable prone and non-prone positions; and
c) a linkage tether connecting the angulation subassembly with the chest slide subassembly; wherein
d) upward breaking of the angulation subassembly translates the chest slide toward the central pivot, and downward breaking of the angulation subassembly translates the chest slide away from the central pivot.

22. The apparatus according to claim 21, further comprising:
a) an infinitely adjustable secondary vertical end elevator connected to and located between a primary vertical end elevator and a respective frame outer end, and adapted for lowering the respective frame outer end an additional distance beyond a distance of lowering provided by the respective primary vertical end elevator.

23. The apparatus according to claim 21, further comprising:
a) a fail-safe apparatus adapted to lock the frame in the event of a catastrophic failure of the apparatus.

24. The apparatus according to claim 21, further comprising:
a) a frame translation compensation mechanism is located within the frame and adapted to translate the frame foot portion away from a connected end elevator in response to upward or downward angling of the frame and to translate the frame foot portion toward the connected end elevator in response to de-angling the frame.

25. The apparatus of claim 21, wherein
a) the central pivot includes a maximum upward breaking position of between about 40° and about 45°.

26. The apparatus of claim 21, wherein
a) the central pivot includes a maximum downward breaking position of about 30°.

27. The apparatus of claim 21, wherein
a) the central pivot includes a total range of motion of about 75°.

28. The apparatus of claim 21, wherein the chest slide subassembly includes
a) a bracket slidingly engaging the frame and attached to the linkage tether; and
b) a sliding chest support releasably engaging the bracket and supported by the frame.

29. The apparatus of claim 28, wherein:
a) the sliding chest support is an imaging table top.

30. An apparatus for supporting a patient during a medical procedure, the apparatus comprising:
a) a breaking patient support subassembly including a head portion and a foot portion joined at a central pivot point;
b) an angulation subassembly including:
i) a pair of opposed hinges, each hinge having first and second knuckles with inboard ends pivotably joined by an upper axle located at the pivot point;
ii) a pair of opposed angulation wedges, each angulation wedge including upper and lower sides, and front and rear ends;
iii) a pair of upper guide members, each of the upper guide members slidably engaging the upper side of one of the angulation wedges, each of the upper guide members having a first through-bore that pivotably receives an associated upper axle therethrough;
iv) a pair of lower guide members, each lower guide member slidably engaging the lower side of the associated angulation wedge, each lower guide member having a second through-bore that pivotably receives an associated lower axle therethrough; and
v) a pair of V-links having inner and outer ends, the lower axles pivotably joining the inner ends with a respective associated lower guide member, and each of the outer ends being pivotably joined with an outboard end of one of the associated first and second knuckles;
c) a torso trolley slidingly supported by the head portion and joined with the angulation subassembly by a linkage tether, the torso trolley adapted for moving in cephalad and caudad directions with respect to the pivot point in response to upward and downward breaking of the patient support subassembly;
d) a pair of selectively telescoping piers supporting the patient support subassembly, the pair of piers including:
i) a head-end pier joined with the outboard end of the head portion and having an independently and continuously adjustable head-end primary elevator; and
ii) a foot-end pier joined with the outboard end of the foot portion and having independently and continuously adjustable foot-end primary and secondary elevators cooperating with the head-end primary elevator;
e) a rotation subassembly cooperating with the head-end and foot-end piers, the elongate patient support subassembly and the angulation subassembly; and
f) a powered actuator for actuating the angulation subassembly and for telescoping the head-end and foot-end piers, so as to so as to allow the hinges to move through an infinitely adjustable non-segmented plurality of angular orientations at the pivot point while substantially maintaining a selected height of the pivot point relative to a floor supporting the apparatus.

31. The apparatus according to claim 30, comprising:
a) a pair of tensioned rear tethers, each tether joining the actuator and an associated angulation wedge, so as to slidingly move the angulation wedges in the cephalad and caudad directions between the associated upper and lower sliding guide members, so as to cause the hinges to break upwardly or downwardly with respect to the floor.

32. The apparatus according to claim 30, the torso trolley comprising:
a) a pair of opposed sliding brackets slidably engaging the head portion of the patient support subassembly;
b) a pair of opposed sliding channel members removably received on the patient support subassembly and each of the sliding channel members releasably mating with a sliding bracket; and
c) a chest slide removably and adjustably received on the pair of sliding channel members.

33. The apparatus according to claim 32, including:
a) a linkage strut pivotably joining each of the sliding brackets with the front end of an associated angulation wedge.

34. The apparatus according to claim 32, wherein:
a) the patient support subassembly includes a frame; and
b) each of the sliding brackets includes an inner surface that defines a trapezoidal cross-section sized and shaped to slidingly mate with the associated frame, wherein the cross-section is taken perpendicular to a longitudinal axis of the frame.

35. The apparatus according to claim 30, further comprising a fail-safe subassembly, the fail-safe subassembly including:
a) a toothed rack associated with the upper side of the angulation wedge and extending from about the front end of the angulation wedge to about the rear end thereof;
b) a pawl pivotably linked with each of the upper sliders and including a ratchet tooth for engaging the toothed rack; wherein
c) in the event of a catastrophic failure of the apparatus, the ratchet tooth engages the toothed rack, so as to block downward breaking of the patient support subassembly subsequent to the failure.

36. The apparatus according to claim 35, wherein:
a) catastrophic failure includes at least one of mechanical failure and electrical failure.

37. The apparatus according to claim 30, wherein:
a) the patient support subassembly includes a frame.

38. The apparatus according to claim 37, wherein:
a) the frame includes a trapezoidal cross-section, wherein the cross-section is taken perpendicular to a longitudinal axis thereof.

39. The apparatus according to claim 30, the patient support subassembly further comprising:
a) a second patient structure, the second structure being a solid support board.

40. The apparatus according to claim 30, the patient support subassembly further comprising:
a) a second patient structure, the second structure being an imaging table.

41. The apparatus according to claim 30, wherein:
a) each angulation wedge includes at least one of an upper locking slot and a lower locking slot, wherein
b) each of the locking slots extends between the front and rear ends thereof; and
c) each of the locking slots being sized and shaped to slidingly receive therein a complementary mating guide key therein.

42. The apparatus according to claim 41, wherein:
a) each of the upper guide members includes an upper guide key sized and shaped to slidingly mate with an associated upper locking slot, so as to slidingly guide the associated upper guide member along the upper side of the associated angulation wedge; and
b) each of the lower guide members includes a lower guide key sized and shaped to slidingly mate with an associated lower locking slot, so as to slidingly guide the associated lower guide member along the lower side of the associated angulation wedge.

43. The apparatus according to claim 42, wherein:
a) each of the upper and lower locking slots includes a trapezoidal cross-section, wherein the cross-section is taken perpendicular to a longitudinal axis thereof.

44. The apparatus of claim 30, wherein the plurality of angular orientations includes a maximum upward breaking position of about 40° to about 45°.

45. The apparatus of claim 30, wherein the plurality of angular orientations includes a maximum downward breaking position of about 30°.

46. The apparatus of claim 30, wherein the plurality of angular orientations includes a total range of motion of about 75°.

47. The apparatus of claim 30, wherein:
a) the apparatus comprises a base; and
b) a portion of an outboard end of the foot portion is lowered below a portion of the base, when the foot-end primary and secondary elevators are maximally lowered.

48. The apparatus according to claim 30, wherein:
a) the rotation subassembly being adapted to rotate the patient support subassembly relative to a longitudinal axis thereof a distance selected from the group consisting of at least about ±5°, at least about ±10°, at least about ±15°, at least about ±20°, at least about ±25°, and at least about ±30°.

49. An apparatus for supporting and positioning a patient during a medical procedure, the apparatus comprising:
a) a pair of spaced opposed hinge subassemblies joining a head portion of an open frame with a foot portion of a frame, the hinge subassemblies being located about the middle of a patient supported on the frame and being adapted to controllably angle in both upwardly and downwardly directions and also to return to a non-angled position; and
b) a hinge driver subassembly cooperating with the hinge subassemblies, the hinge driver subassembly being translated in a cephalad direction when the hinge subassemblies angle downwardly and also being translated in a caudad direction when the hinge subassemblies angle upwardly, so as to manipulate a patient supported on the frame in a plurality of prone and non-prone positions.

50. An apparatus for supporting and positioning a patient during a medical procedure, comprising:
a) a prone patient support structure suspended above a floor and adapted for selectively and reversibly flexing and articulating the spine of a patient supported thereon while maintaining the spine at selected height;
b) a pair of first vertical translation subassemblies held in fixed spaced opposed relation to one another; each of the first vertical translation subassemblies including a pitch axis and being reversibly joined with an outboard end of the prone patient support structure so as to allow for reversible rotational movement of the respective outboard end about the respective pitch axis.

51. The apparatus according to claim 1, wherein:
a) the spaced apart hinges are gearless.

52. The apparatus according to claim 1, wherein:
a) each of the spaced apart hinges includes a cam.

53. The apparatus according to claim 1, wherein:
a) each of the spaced apart hinges includes a push-pull control rod.

54. The apparatus according to claim 1, wherein:
a) each of the spaced apart hinges includes an angulation wedge actuated by a push-pull control rod.

55. The apparatus according to claim 3, wherein:
a) the spaced apart hinges are gearless.

56. The apparatus according to claim 3, wherein:
a) the hinge driver subassembly includes a cam.

57. The apparatus according to claim 3, wherein:
a) the hinge driver subassembly includes a push-pull control rod.

58. The apparatus according to claim 3, wherein:
a) each of the spaced apart hinges includes an angulation wedge actuated by a push-pull control rod.

59. The assembly according to claim 7, wherein:
a) the translation compensation subassembly includes a sliding inner translation bar that is coplanar with the portion of the frame surrounding the inner translation bar.

60. The apparatus according to claim 12, wherein:
a) the spaced apart hinges are gearless.

61. The apparatus according to claim 12, wherein:
a) the hinge driver subassembly includes a cam.

62. The apparatus according to claim 12, wherein:
a) the hinge driver subassembly includes a push-pull control rod.

63. The apparatus according to claim 12, wherein:
a) each of the spaced apart hinges includes an angulation wedge actuated by a push-pull control rod.

64. The assembly according to claim 13, wherein:
a) the translation compensation subassembly includes a sliding inner translation bar that is coplanar with the portion of the frame surrounding the inner translation bar.

65. The apparatus according to claim 21, wherein:
a) the spaced apart hinges are gearless.

66. The apparatus according to claim 21, wherein:
a) the hinge driver subassembly includes a cam.

67. The apparatus according to claim 21, wherein:
a) the hinge driver subassembly includes a push-pull control rod.

68. The apparatus according to claim 21, wherein:
a) each of the spaced apart hinges includes an angulation wedge actuated by a push-pull control rod.

69. The assembly according to claim 24, wherein:
a) the translation compensation subassembly includes a sliding inner translation bar that is coplanar with the portion of the frame surrounding the inner translation bar.

70. The apparatus according to claim 30, wherein:
a) the spaced apart hinges are gearless.

71. The apparatus according to claim 30, wherein:
a) the hinge driver subassembly includes a cam.

72. The apparatus according to claim 30, wherein:
a) the hinge driver subassembly includes a push-pull control rod.

73. The apparatus according to claim 30, wherein:
a) each of the spaced apart hinges includes an angulation wedge actuated by a push-pull control rod.

74. The assembly according to claim 30, further comprising:
a) a translation compensation subassembly with a sliding inner translation bar that is coplanar with a portion of the frame surrounding the inner translation bar.

75. The apparatus according to claim 49, wherein:
a) the hinge subassemblies are gearless.

76. The apparatus according to claim 49, wherein:
a) the hinge driver subassembly includes a cam.

77. The apparatus according to claim 49, wherein:
a) each of the hinge driver subassemblies includes a rotatable elongate member.

78. The apparatus according to claim 49, wherein:
a) each of the hinge subassemblies includes an angulation wedge actuated by a push-pull control rod.

79. The assembly according to claim 49, further comprising:
a) a translation compensation subassembly with a sliding inner translation bar that is coplanar with a portion of the frame surrounding the inner translation bar.

80. The apparatus according to claim 1, wherein:
a) when the hinges break upwardly, movement of the linkage in one direction causes the chest slide to translate in the same direction.

81. An apparatus for supporting a patient during a medical procedure, the apparatus comprising:
a) an open frame having a single breaking location around the middle of a patient positioned on the frame, the frame being outwardly connected to and supported between spaced apart head and foot primary motorized vertical end elevators; and
b) an additional motorized vertical end elevator adapted for infinite adjustability to better align and orient the frame so as to allow for additional vertical adjustability of a frame outer end beyond an amount provided by the primary elevators; wherein
c) the frame can controllably break and bend, upwardly and downwardly, and is adapted to manipulate the patient in a plurality of selectively lockable prone and non-prone positions in cooperation with a frame translation compensation mechanism located within an end of the frame, while also cooperating with the primary and secondary motorized end elevators to move the patient vertically while the patient is positioned on the frame.

82. An apparatus for supporting and positioning a patient during a medical procedure, comprising:
a) a prone patient support structure suspended above a floor and adapted for selectively and reversibly flexing and articulating the spine of a patient supported thereon while maintaining the spine at selected height above a floor; and
b) the patient support structure including head and foot end sections forming a frame with outer and inner ends, the inner ends being connected at a pair of hinges and the outer ends being connected to first and second vertical translation subassemblies; wherein
c) the first and second vertical translation subassemblies are held in fixed spaced opposed relation to one another; each of the vertical translation subassemblies including pitch and yaw axes about which the outer ends of the prone patient support structure are rotatable.
